(19) United States
(12) Patent Application Publication
(10) Pub. No.: US 2024/0199701 A1
(43) Pub. Date: Jun. 20, 2024

(54) IMMUNOMODULATORS

(71) Applicant: BRISTOL-MYERS SQUIBB COMPANY, PRINCETON, NJ (US)

(72) Inventors: JENNIFER X. QIAO, ARLINGTON, MA (US); MICHAEL A. POSS, LAWRENCEVILLE, NJ (US); YUNHUI ZHANG, PRINCETON, NJ (US); MARTIN PATRICK ALLEN, FLEMINGTON, NJ (US)

(73) Assignee: BRISTOL-MYERS SQUIBB COMPANY, PRINCETON, NJ (US)

(21) Appl. No.: 18/551,319

(22) PCT Filed: Mar. 24, 2022
(86) PCT No.: PCT/US2022/021760

§ 371 Date: Sep. 19, 2023

Related U.S. Application Data
(60) Provisional application No. 63/165,455, filed on Mar. 24, 2021.

(51) Int. Cl. C07K 7/54; A61K 38/00; A61K 47/54

(52) U.S. Cl. CPC C07K 7/54; A61K 47/542; A61K 47/548; A61K 38/00

(57) ABSTRACT

The present disclosure provides novel macrocyclic peptides which inhibit the PD-1/PD-L1 and PD-L1/CD80 protein/protein interaction, and thus are useful for the amelioration of various diseases, including cancer and infectious diseases.

### CROSS REFERENCE TO RELATED APPLICATIONS

This application claims priority to U.S. Provisional Application No. 63/165,455, filed Mar. 24, 2021, which is incorporated by reference in its entirety.

### REFERENCE TO SEQUENCE LISTING SUBMITTED ELECTRONICALLY

The content of the electronically submitted sequence listing in ASCII text file (Name 3338_281PC01_Seqlisting_ST25; Size 5,923 bytes; and Date of Creation: Mar. 23, 2022) filed with the application is incorporated herein by reference in its entirety.

### FIELD

The present disclosure provides macrocyclic compounds that bind to PD-L1 and are capable of inhibiting the interaction of PD-L1 with PD-1 and CD80. These macrocyclic compounds exhibit in vitro immunomodulatory efficacy thus making them therapeutic candidates for the treatment of various diseases including cancer and infectious diseases.

### BACKGROUND

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells.

The PD-1 protein is a 55 kDa type I transmembrane protein that is part of the Ig gene superfamily. PD-1 contains a membrane proximal immunoreceptor tyrosine inhibitory motif (ITIM) and a membrane distal tyrosine-based switch motif. Although structurally similar to CTLA-4, PD-1 lacks the MYPPY motif that is critical for CD80 CD86 (B7-2) binding. Two ligands for PD-1 have been identified, PD-L1 (B7-H1) and PD-L2 (b7-DC). The activation of T cells expressing PD-1 has been shown to be downregulated upon interaction with cells expressing PD-L1 or PD-L2. Both PD-L1 and PD-L2 are B7 protein family members that bind to PD-1, but do not bind to other CD28 family members. The PD-L1 ligand is abundant in a variety of human cancers. The interaction between PD-1 and PD-L1 results in a decrease in tumor infiltrating lymphocytes, a decrease in T-cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

PD-L1 has also been shown to interact with CD80. The interaction of PD-L1/CD80 on expressing immune cells has been shown to be an inhibitory one. Blockade of this interaction has been shown to abrogate this inhibitory interaction.

When PD-1 expressing T cells contact cells expressing its ligands, functional activities in response to antigenic stimuli, including proliferation, cytokine secretion, and cytotoxicity, are reduced. PD-1/PD-L1 or PD-L2 interactions down regulate immune responses during resolution of an infection or tumor, or during the development of self. Chronic antigen stimulation, such as that which occurs during tumor disease or chronic infections, results in T cells that express elevated levels of PD-1 and are dysfunctional with respect to activity towards the chronic antigen. This is termed “T cell exhaustion”. B cells also display PD-1/PD-ligand suppression and “exhaustion”.

Blockade of PD-1/PD-L1 ligation using antibodies to PD-L1 has been shown to restore and augment T cell activation in many systems. Patients with advanced cancer benefit from therapy with a monoclonal antibody to PD-L1. Preclinical animal models of tumors and chronic infections have shown that blockade of the PD-1/PD-L1 pathway by monoclonal antibodies can enhance an immune response and result in tumor rejection or control of infection. Antitumor immunotherapy via PD-1/PD-L1 blockade can augment therapeutic immune response to a number of histologically distinct tumors.

Interference with the PD-1/PD-L1 interaction causes enhanced T cell activity in systems with chronic infection. Blockade of PD-L1 caused improved viral clearance and restored immunity in mice with chromoic lymphocytic chorio meningitis virus infection. Humanized mice infected with HIV-1 show enhanced protection against viremia and viral depletion of CD4+ T cells. Blockade of PD-1/PD-L1 through monoclonal antibodies to PD-L1 can restore in vitro antigen-specific functionality to T cells from HIV patients.

Blockade of the PD-L1/CD80 interaction has also been shown to stimulate immunity. Immune stimulation resulting from blockade of the PD-L1/CD80 interaction has been shown to be enhanced through combination with blockade of further PD-1/PD-L1 or PD-1/PD-L2 interactions.

Alterations in immune cell phenotypes are hypothesized to be an important factor in septic shock. These include increased levels of PD-1 and PD-L1. Cells from septic shock patients with increased levels of PD-1 and PD-L1 exhibit an increased level of T cell apoptosis. Antibodies directed to PD-L1, can reduce the level of immune cell apoptosis. Furthermore, mice lacking PD-1 expression are more resistant to septic shock symptoms than wildtype mice. Studies have revealed that blockade of the interactions of PD-L1 using antibodies can suppress inappropriate immune responses and ameliorate disease signs.

In addition to enhancing immunologic responses to chronic antigens, blockade of the PD-1/PD-L1 pathway has also been shown to enhance responses to vaccination, including therapeutic vaccination in the context of chronic infection.

The PD-1 pathway is a key inhibitory molecule in T cell exhaustion that arises from chronic antigen stimulation during chronic infections and tumor disease. Blockade of the PD-1/PD-L1 interaction through targeting the PD-L1 protein has been shown to restore antigen-specific T cell immune functions in vitro and in vivo, including enhanced responses to vaccination in the setting of tumor or chronic infection. Accordingly, agents that block the interaction of PD-L1 with either PD-1 or CD80 are desired.

### SUMMARY

The present disclosure provides macrocyclic compounds which inhibit the PD-1/PD-L1 and CD80/PD-L1 protein/protein interaction, and are thus useful for the amelioration of various diseases, including cancer and infectious diseases.

In its first embodiment the present disclosure provides a compound of formula (I)

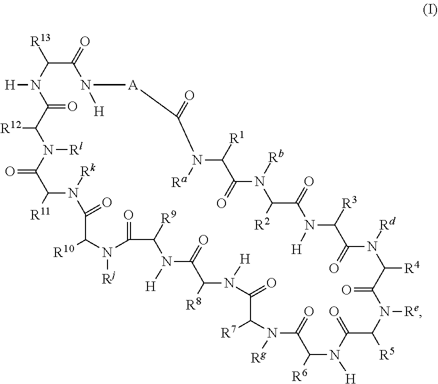

or a pharmaceutically acceptable salt thereof, wherein:
A is selected from

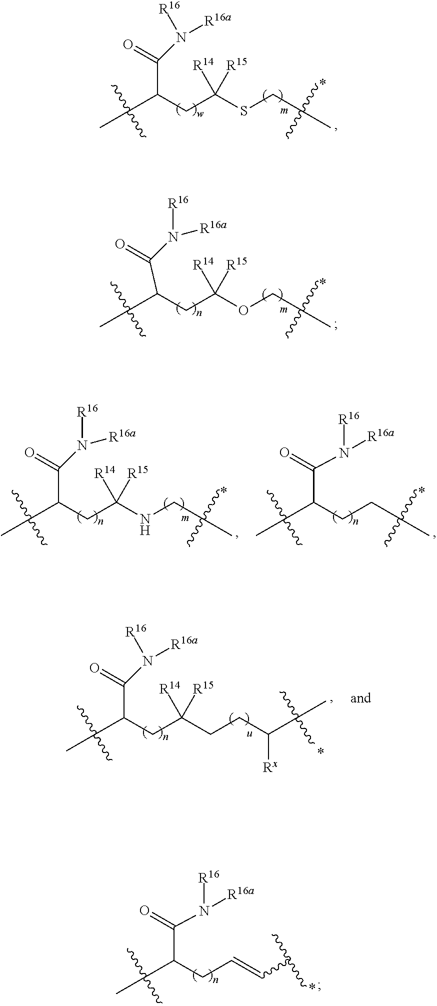

wherein:
[symbol] denotes the point of attachment to the carbonyl group and [symbol] denotes the point of attachment to the nitrogen atom;
n is 0 or 1;
m is 1 or 2;
u is 0 or 1;
w is 0, 1, or 2;
R^{x} is selected from hydrogen, amino, hydroxy, and methyl;
R¹⁴ and R¹⁵ are independently selected from hydrogen and methyl;
R^{16a} is selected from hydrogen and C₁-C₆ alkyl;
R¹⁶ is selected from
—(C(R^{17a})₂)₂—X—R³⁰, —(C(R^{17a}R¹⁷))₀₋₂—X′—R³⁰, —(C(R^{17a}R¹⁷)₁₋₂C(O)NR^{16a})_{m′}—X′—R³⁰,
—C(R^{17a})₂C(O)N(R^{16a})C(R^{17a})₂—X′—R³¹, —(C(R^{17a}R¹⁷))₁₋₂C(O)N(R^{16a})C(R^{17a})₂—X′—R³¹,
—C(R^{17a})₂[C(O)N(R^{16a})C(R^{17a})₂]_{w′}—X—R³¹, —C(R^{17a}R¹⁷)₁₋₂[C(O)N(R^{16a})C(R^{17a}R¹⁷)₁₋₂]_{w′}—X′—R³¹,
—(C(R^{17a})(R¹⁷)C(O)NR^{16a})_{n′}—H; and
—(C(R^{17a})(R¹⁷)C(O)NR^{16a})_{m′}—C(R^{17a})(R¹⁷)—CO₂H;
   wherein a PEG_{q′} spacer can be inserted in any part of the R¹⁶ (q′ is the number of —(CH₂CH₂O)— unit in a PEG spacer);
   wherein w′ is 2 or 3;
n′ is 1-6;
m′ is 0-5;
q′ is 1-20
X is a chain of between 1 and 172 atoms wherein the atoms are selected from nitrogen, carbon and oxygen and wherein the chain may contain one, two, three, or four groups selected from —NHC(O)—, —NHC(O)NH—, and —C(O)NH— embedded therein; and wherein the chain is optionally substituted with one to six groups independently selected from —CO₂H, —C(O)NH₂, —CH₂C(O)NH₂, and —(CH₂)₁₋₂CO₂H;
X′ is a chain of between 1 and 172 atoms wherein the atoms are selected from carbon and oxygen and wherein the chain may contain one, two, three, or four groups selected from —NHC(O)—, —NHC(O)NH—, and —C(O)NH— embedded therein; and wherein the chain is optionally substituted with one to six groups independently selected from —CO₂H, —C(O)NH₂, and —(CH₂)₁₋₂CO₂H, provided that X′ is other than unsubstituted PEG;
R³⁰ is selected from —SO₃H, —S(O)OH, and —P(O)(OH)₂;
R³¹ is selected from —S(O)₂OH, —S(O)OH, and —P(O)(OH)₂;
each R^{17a} is independently selected from hydrogen, C₁-C₆alkyl, —CH₂OH, —CH₂CO₂H, —(CH₂)₂CO₂H,
each R¹⁷ is independently selected from hydrogen, —CH₃, (CH₂)_{z}N₃, —(CH₂)_{z}NH₂, —X—R³¹, —(CH₂)_{z}CO₂H, —CH₂OH, CH₂C≡CH, and —(CH₂)_{z}-triazolyl-X—R³⁵, wherein z is 1-6 and R³⁵ is selected from —SO₃H, —S(O)OH, and —P(O)(OH)₂; provided at least one R¹⁷ is other than hydrogen, —CH₃, or —CH₂OH;
   provided at least one of R³⁰, R³¹ or R³⁵ is present;
R^{a}, R^{e}, R^{j}, and R^{k}, are each independently selected from hydrogen and methyl;
R¹, R², R³, R⁴, R⁵, R⁶, R⁷, R⁸, R⁹, R¹⁰, R¹¹, R¹², and R¹³ are independently selected from a natural amino acid side chain and an unnatural amino acid side chain or form a ring with the corresponding vicinal R group as described below;
R^{b} is methyl or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{d} is hydrogen or methyl, or, R^{d} and R⁴, together with the atoms to which they are attached, can form a ring selected from azetidine, pyrrolidine, morpholine, piperidine, piperazine, and tetrahydrothiazole; wherein each ring is optionally substituted with one to four groups independently selected from amino, cyano, methyl, halo, hydroxy, and phenyl;
R⁹ is hydrogen or methyl or R⁹ and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R¹ is methyl or, R¹ and R¹², together with the atoms to which they are attached, form a ring selected from azetidine and pyrrolidine, wherein each ring is optionally substituted with one to four independently selected from amino, cyano, methyl, halo, and hydroxy.

In a first aspect of the first embodiment the present disclosure provides a compound of formula (I), or a pharmaceutically acceptable salt thereof, wherein A is

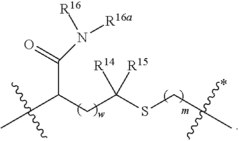

In a second aspect of the first embodiment:
m is 1 and w is 0; and
R¹⁴, R¹⁵, and R^{16a} are each hydrogen.

In a third aspect of the first embodiment:
R¹⁶ is selected from —(C(R^{17a})₂)₂—X—R³⁰, —(C(R^{17a}R¹⁷))₀₋₂—X′—R³⁰ and —(C(R^{17a}R¹⁷)₁₋₂C(O)NR^{16a})_{m′}—X′—R³⁰,

In a fourth aspect of the first embodiment:
each R^{17a} is selected from hydrogen, —CO₂H, and —CH₂CO₂H;
X is a chain of between 8 and 46 atoms wherein the atoms are selected from carbon and oxygen and wherein the chain may contain one, two, or three —NHC(O)—, C(O)NH groups embedded therein; and wherein the chain is optionally substituted with one or two groups independently selected from —CO₂H, —C(O)NH₂, —CH₂C(O)NH₂, and —CH₂CO₂H; and
R³⁰ is selected from —SO₃H and —P(O)(OH)₂;

In a fifth aspect of the first embodiment the present disclosure provides a compound of formula (I), or a pharmaceutically acceptable salt thereof, wherein:
A is

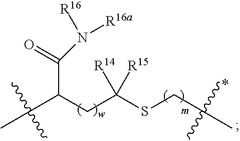

m and w are 1;
R¹⁴, R¹⁵, and R^{16a} are each hydrogen; and
R¹⁶ is selected from —C(R^{17a})₂C(O)N(R^{16a})C(R^{17a})₂—X′—R³¹ and —(C(R^{17a}R¹⁷))₁₋₂C(O)N(R^{16a})C(R^{17a})₂—X′—R³¹,
In a sixth aspect of the first embodiment:
each R^{17a} is selected from hydrogen, —CO₂H, and —CH₂CO₂H;
X′ is a chain of between 8 and 48 atoms wherein the atoms are selected from carbon and oxygen and wherein the chain may contain one, two, or three —NHC(O)— or —C(O)NH— groups embedded therein; and wherein the chain is optionally substituted with one or two groups independently selected from —CO₂H, —C(O)NH₂, —CH₂C(O)NH₂, and —CH₂CO₂H; provided that X′ is other than unsubstituted PEG; and
R³⁰ is selected from —SO₃H and —P(O)(OH)₂.

In a seventh aspect of the first embodiment the present disclosure provides a compound of formula (I), or a pharmaceutically acceptable salt thereof, wherein:
A is

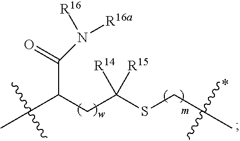

m is 1 and w is 0;
R¹⁴, R¹⁵, and R^{16a} are each hydrogen; and
R¹⁶ is selected from —C(R^{17a})₂[C(O)N(R^{16a})C(R^{17a})₂]_{w′}—X—R³¹ and —C(R^{7a}R⁷)₁₋₂[C(O)N(R^{16a})C(R^{17a}R¹⁷)₁₋₂]_{w′}—X′—R³¹,

In an eighth aspect of the first embodiment:

each R^{17a} is selected from hydrogen, —CO₂H, and —CH₂CO₂H;
X is a chain of between 8 and 48 atoms wherein the atoms are selected from carbon and oxygen and wherein the chain may contain one, two, or three —NHC(O)— or —C(O)NH— groups embedded therein; and wherein the chain is optionally substituted with one or two groups independently selected from —CO₂H, —C(O)NH₂, —CH₂C(O)NH₂, and —CH₂CO₂H; and
R³¹ is selected from —SO₃H and —P(O)(OH)₂.

In a ninth aspect of the first embodiment the present disclosure provides a compound of formula (I), or a pharmaceutically acceptable salt thereof, wherein:
A is

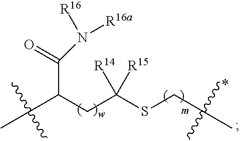

m is 1 and w is 0;
R¹⁴, R¹⁵, and R^{16a} are each hydrogen; and
R¹⁶ is —(C(R^{17a})(R¹⁷)C(O)NR^{16a})_{n′}—H.

In a tenth aspect of the first embodiment:
each R^{17a} is hydrogen; and
each R¹⁷ is selected from hydrogen, —CH₃, (CH₂)_{z}N₃, —(CH₂)_{z}NH₂, —X—R³¹, —(CH₂)_{z}CO₂H, —CH₂OH, CH₂C≡CH, and —(CH₂)_{z}-triazolyl-X—R³⁵; provided at least one R¹⁷ is other than hydrogen, —CH₃, or —CH₂OH, and provided at least one R³¹ or R³⁵ is present;
z is 1-4;
R³¹ is selected from —SO₃H and —P(O)(OH)₂;
X is a chain of between 7 and 155 atoms wherein the atoms are selected from carbon and oxygen and wherein the chain may contain one, two, or three —NHC(O)—, or —C(O)NH— groups embedded therein; and wherein the chain is optionally substituted with one or two groups independently selected from —CO₂H, —C(O)NH₂, —CH₂C(O)NH₂, and —CH₂CO₂H; and
   R³⁵ is selected from —SO₃H and —P(O)(OH)₂.

In an eleventh aspect of the first embodiment the present disclosure provides a compound of formula (I), or a pharmaceutically acceptable salt thereof, wherein:
A is

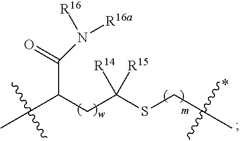

m is 1 and w is 0;
R¹⁴, R¹⁵, and R^{16a} are each hydrogen; and
R¹⁶ is —(CR^{17a})(R¹⁷)C(O)NR^{16a})_{m′}—C(R^{17a})(R¹⁷)—CO₂H.

In a twelfth aspect of the first embodiment:
m′ is 0-3;
each R^{17a} is hydrogen;
each R¹⁷ is selected from hydrogen, —CH₃, (CH₂)_{z}N₃, —(CH₂)_{z}NH₂, —X—R³¹, —(CH₂)_{z}CO₂H, —CH₂OH, CH₂C≡CH, and —(CH₂)_{z}-triazolyl-X—R³⁵; provided at least one R¹⁷ is other than hydrogen, —CH₃, or —CH₂OH, and provided at least one R³¹ or R³⁵ is present;
z is 1-4;
R³¹ is selected from —SO₃H and —P(O)(OH)₂;
X is a chain of between 10 and 60 atoms wherein the atoms are selected from carbon and oxygen and wherein the chain may contain one, two, or three —NHC(O)—, —C(O)NH— groups embedded therein; and wherein the chain is optionally substituted with one or two groups independently selected from —CO₂H, —C(O)NH₂, —CH₂C(O)NH₂, and —CH₂CO₂H; and
R³⁵ is selected from —SO₃H and —P(O)(OH)₂.

In a thirteenth aspect of the first embodiment the present disclosure provides a compound of formula (I), or a pharmaceutically acceptable salt thereof, wherein R¹ is phenylC₁-C₃alkyl wherein the phenyl part is optionally substituted with hydroxyl, halo, or methoxy; R² is C₁-C₇alkyl or, R² and R^{b}, together with the atoms to which they are attached, form a piperidine ring; R³ is NR^{x}R^{y}(C₁-C₇alkyl), NR^{u}R^{v}carbonylC₁-C₃alkyl, or carboxyC₁-C₃alkyl; R⁴ and R^{d}, together with the atoms to which they are attached, form a pyrrolidine ring; R⁵ is hydroxyC₁-C₃alkyl, imidazolylC₁-C₃alkyl, or NR^{x}R^{y}(C₁-C₇alkyl); R⁶ is carboxyC₁-C₃alkyl, NR^{uR}carbonylC₁-C₃alkyl, NR^{x}R^{y}(C₁-C₇alkyl), or C₁-C₇alkyl; R⁷ and R⁹, together with the atoms to which they are attached, form a pyrrolidine ring optionally substituted with hydroxy; R⁸ and R¹⁰ are benzothienyl or indolylC₁-C₃alkyl optionally substituted with carboxyC₁-C₃alkyl; R⁹ is hydroxyC₁-C₃alkyl, aminoC₁-C₄alkyl, or C₁-C₇alkyl, R¹¹ is C₁-C₃alkoxyC₁-C₃alkyl or C₁-C₇alkyl; R¹² is C₁-C₇alkyl or hydroxyC₁-C₃alkyl; and R¹³ is C₁-C₇ alkyl, carboxyC₁-C₃alkyl, or —(CH₂)₃NHC(NH)NH₂.

In a fourteenth aspect of the first embodiment the present disclosure provides a compound of formula (I), or a pharmaceutically acceptable salt thereof, wherein:
A is

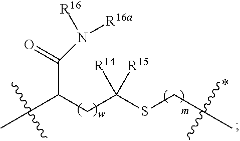

m is 1 and w is 0;
R¹⁴ and R¹⁵, are each hydrogen;
R^{16a} is hydrogen or methyl;
R^{d} is methyl or, R^{d} and R⁴, together with the atoms to which they are attached, form a ring selected from azetidine, pyrrolidine, morpholine, piperidine, piperazine, and tetrahydrothiazole; wherein each ring is optionally substituted with one or two groups independently selected from amino, cyano, methyl, halo, hydroxy, and phenyl; and
R⁹ is methyl or, R⁹ and R⁷, together with the atoms to which they are attached, form a ring selected from azetidine, pyrrolidine, morpholine, piperidine, piperazine, and tetrahydrothiazole; wherein each ring is optionally substituted with one or two groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group.

In a second embodiment the present disclosure provides a compound of formula (II)

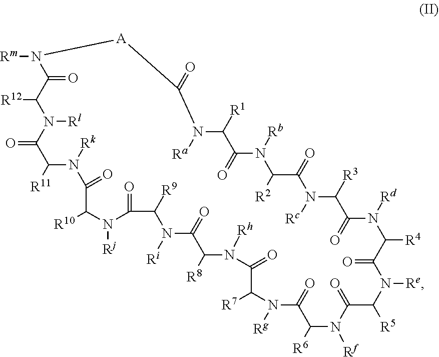

or a pharmaceutically acceptable salt thereof, wherein:
A is selected from

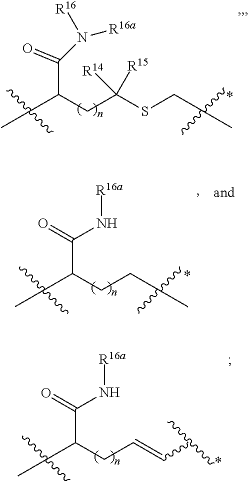

wherein:
n is 0 or 1;
R¹⁴ and R¹⁵ are independently selected from hydrogen and methyl;
R^{16a} is selected from hydrogen and C₁-C₆ alkyl;
R¹⁶ is selected from
—(C(R^{17a})₂)₂—X—R³⁰, —(C(R^{17a}R¹⁷))₀₋₂—X′—R³⁰, —(C(R^{17a}R¹⁷)₁₋₂C(O)NR^{16a})_{m′}—X′—R³⁰,
—C(R^{17a})₂C(O)N(R^{16a})C(R^{17a})₂—X′—R³¹, —(C(R^{17a}R¹⁷))₁₋₂C(O)N(R^{16a})C(R^{17a})₂—X′—R³¹,
—C(R^{17a})₂[C(O)N(R^{16a})C(R^{17a})₂]_{w′}—X—R³¹, —C(R^{17a}R¹⁷)₁₋₂[C(O)N(R^{16a})C(R^{17a}R¹⁷)₁₋₂]_{w′}—X′—R³¹, —(C(R^{17a})(R¹⁷)C(O)NR^{16a})_{n′}—H; and —(C(R^{17a})(R¹⁷)C(O)NR^{16a})_{m′}—C(R^{17a})(R¹⁷)—CO₂H;
   wherein a PEG_{q′} spacer can be inserted in any part of the R¹⁶ (q′ is the number of —(CH₂CH₂O)— unit in a PEG spacer);
   wherein:
w′ is 2 or 3;
n′ is 1-6;
m′ is 0-5;
q′ is 1-20
X is a chain of between 1 and 172 atoms wherein the atoms are selected from carbon and oxygen and wherein the chain may contain one, two, three, or four groups selected from —NHC(O)—, —NHC(O)NH—, and —C(O)NH embedded therein; and wherein the chain is optionally substituted with one to six groups independently selected from —CO₂H, —C(O)NH₂, —CH₂C(O)NH₂, and —CH₂CO₂H,
X′ is a chain of between 1 and 172 atoms wherein the atoms are selected from carbon and oxygen and wherein the chain may contain one, two, three, or four groups selected from —NHC(O)—, —NHC(O)NH—, and —C(O)NH embedded therein; and wherein the chain is optionally substituted with one to six groups independently selected from —CO₂H, —C(O)NH₂, and —CH₂CO₂H, provided that X′ is other than unsubstituted PEG;
R³⁰ is selected from —SO₃H, —S(O)OH, and —P(O)(OH)₂;
R³¹ is —SO₃H, —S(O)OH, and —P(O)(OH)₂;
each R^{17a} is independently selected from hydrogen, C₁-C₆alkyl, —CH₂OH, —CH₂CO₂H, —(CH₂)₂CO₂H,
each R¹⁷ is independently selected from hydrogen, —CH₃, (CH₂)_{z}N₃, —(CH₂)_{z}NH₂, —X—R³¹, —(CH₂)_{z}CO₂H, —CH₂OH, CH₂C≡CH, and —(CH₂)_{z}-triazolyl-X—R³⁵, wherein z is 1-6 and R³⁵ is selected from —S(O)₂OH, —S(O)OH, and —P(O)(OH)₂;
   provided at least one R¹⁷ is other than hydrogen, —CH₃, or —CH₂OH;
   provided at least one of R³⁰, R³¹ or R³⁵ is present; R^{a}, R^{f}, R^{j}, R^{k}, R^{l}, and R^{m} are hydrogen;
R^{b} and R^{c} are methyl;
R^{g} is selected from hydrogen and methyl;
R¹, R², R³, R⁴, R⁵, R⁶, R⁷, R⁸, R⁹, R¹⁰, R¹¹, and R¹² are independently selected from a natural amino acid side chain and an unnatural amino acid side chain or form a ring with the corresponding vicinal R group as described below;
R^{d} is selected from hydrogen and methyl, or, R^{d} and R⁴, together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, halomethyl, and hydroxy;
R^{e} is selected from hydrogen and methyl, or, R^{e} and R⁵, together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, halomethyl, and hydroxy;
R^{h} is selected from hydrogen and methyl, or, R^{h} and R⁸, together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, halomethyl, and hydroxy; and
Rⁱ is selected from hydrogen and methyl, or, Rⁱ and R⁹, together with the atoms to which they are attached selected from azetidine, pyrrolidine, morpholine, piperidine, piperazine, and tetrahydrothiazole; wherein each ring is optionally substituted with one to four groups independently selected from amino, cyano, methyl, halo, halomethyl, and hydroxy.

In a first aspect of the second embodiment the present disclosure provides a compound of formula (II), or a pharmaceutically acceptable salt thereof, wherein
A is

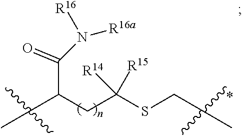

n is 0;
R¹⁶ is —(CR^{17a})(R¹⁷)C(O)NR^{16a})_{m′}—C(R^{17a})(R¹⁷)—CO₂H;
each R^{16a} is hydrogen;
m′ is 2, 3, or 4;
each R^{17a} is hydrogen;
each R¹⁷ is independently selected from hydrogen, —(CH₂)_{z}NH₂, —X—R³¹ and —CH₂C≡CH,
z is 4;
X is a chain of between 26 and 155 atoms wherein the atoms are selected from carbon and oxygen and wherein the chain may contain one, two, or three —NHC(O)— or —C(O)NH— groups embedded therein; and wherein the chain is optionally substituted with one or two groups independently selected from —CO₂H, —C(O)NH₂, —CH₂C(O)NH₂, and —CH₂CO₂H; and
R³¹ is —S(O)₂OH and —P(O)(OH)₂.

In a third embodiment the present disclosure provides a method of enhancing, stimulating, and/or increasing an immune response in a subject in need thereof, said method comprising administering to the subject a therapeutically effective amount of a compound of formula (I) or a pharmaceutically acceptable salt thereof. In a first aspect of the third embodiment the method further comprises administering an additional agent prior to, after, or simultaneously with the compound of formula (I) or a pharmaceutically acceptable salt thereof. In a second aspect the additional agent is an antimicrobial agent, an antiviral agent, a cytotoxic agent, and/or an immune response modifier.

In a fourth embodiment the present disclosure provides a method of inhibiting growth, proliferation, or metastasis of cancer cells in a subject in need thereof, said method comprising administering to the subject a therapeutically effective amount a compound of formula (I), or a pharmaceutically acceptable salt thereof. In a first aspect of the fourth embodiment the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

In a fifth embodiment the present disclosure provides a method of treating an infectious disease in a subject in need thereof, the method comprising administering to the subject a therapeutically effective amount of a compound of formula (I) or a pharmaceutically acceptable salt thereof. In a first aspect of the fifth embodiment the infectious disease is caused by a virus. In a second aspect the virus is selected from HIV, Hepatitis A, Hepatitis B, Hepatitis C, herpes viruses, and influenza.

In a sixth embodiment the present disclosure provides a method of treating septic shock in a subject in need thereof, the method comprising administering to the subject a therapeutically effective amount of a compound of formula (I) or a pharmaceutically acceptable salt thereof.

In a seventh embodiment the present disclosure provides a method of enhancing, stimulating, and/or increasing an immune response in a subject in need thereof, said method comprising administering to the subject a therapeutically effective amount of a compound of formula (II) or a pharmaceutically acceptable salt thereof. In a first aspect of the seventh embodiment the method further comprises administering an additional agent prior to, after, or simultaneously with the compound of formula (II) or a pharmaceutically acceptable salt thereof. In a second aspect the additional agent is an antimicrobial agent, an antiviral agent, a cytotoxic agent, and/or an immune response modifier. In a third aspect the additional agent is an HDAC inhibitor. In a fourth embodiment the additional agent is a TLR7 and/or TLR8 agonist. In another embodiment, the additional agent is a STING, NLRP3 or DGK agent.

In an eighth embodiment the present disclosure provides a method of inhibiting growth, proliferation, or metastasis of cancer cells in a subject in need thereof, said method comprising administering to the subject a therapeutically effective amount a compound of formula (II), or a pharmaceutically acceptable salt thereof. In a first aspect of the eighth embodiment the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

In a ninth embodiment the present disclosure provides a method of treating an infectious disease in a subject in need thereof, the method comprising administering to the subject a therapeutically effective amount of a compound of formula (II) or a pharmaceutically acceptable salt thereof. In a first aspect of the ninth embodiment the infectious disease is caused by a virus. In a second aspect the virus is selected from HIV, Hepatitis A, Hepatitis B, Hepatitis C, herpes viruses, and influenza.

In a tenth embodiment the present disclosure provides a method of treating septic shock in a subject in need thereof, the method comprising administering to the subject a therapeutically effective amount of a compound of formula (II) or a pharmaceutically acceptable salt thereof.

In compounds of formula (I) where the R side chains are part of a ring that is substituted with methyl, it is understood that the methyl group may be on any substitutable carbon atom in the ring, including the carbon that is part of the macrocyclic parent structure.

The following groups are preferred at each R position. The amino acids may be D- or L-stereochemistry and may be substituted as described elsewhere in the disclosure.

In compounds of formula (I), preferred R¹ is selected from the side chains of: phenylalanine, tyrosine, 3-thien-2-yl, 4-methylphenylalanine, 4-chlorophenylalanine, 3-methoxyphenylalananie, isotryptophan, 3-methylphenylalanine, 1-naphthylalanine, 3,4-difluorophenylalanine, 4-fluorophenylalanine, 3,4-dimethoxyphenylalanine, 3,4-dichlorophenylalanine, 4-difluoromethylphenylalanine, 2-methylphenylalanine, 2-naphthylalanine, tryptophan, 4-pyridinyl, 4-bromophenylalanine, 3-pyridinyl, 4-trifluoromethylphenylalanine, 4-carboxyphenylalanine, 4-methoxyphenylalanine, biphenylalanine, and 3-chlorophenylalanine; and 2,4-diaminobutane.

In compounds of formula (I) where R² is not part of a ring, preferred R² is selected from the side chains of alanine, serine, and glycine.

In compounds of formula (I), preferred R³ is selected from the side chains of: asparagine, aspartic acid, glutamic acid, glutamine, serine, ornithine (Orn), lysine, histidine, threonine, leucine, alanine, Dap, and Dab.

In compounds of formula (I) where R⁴ is not part of a ring, preferred R⁴ is selected from the side chains of valine, alanine, isoleucine, and glycine.

In compounds of formula (I), preferred R⁵ is selected from the side chains of histidine, asparagine, Dap, Dap(COCH₃), serine, glycine, Dab, Dab(COCH₃), alanine, lysine, aspartic acid, alanine, and 3-thiazolylalanine.

In compounds of formula (I), preferred R⁶ is selected from the side chains of leucine, aspartic acid, asparagine, glutamic acid, glutamine, serine, lysine, 3-cyclohexane, threonine, ornithine, Dab, alanine, arginine, and Orn(COCH₃).

In compounds of formula (I) where R⁷ is not part of a ring, preferred R⁷ is selected from the side chains of glycine, Dab, serine, lysine, arginine, ornithine, histidine, asparagine, glutamine, alanine, and Dab (C(O)cyclobutane).

In compounds of formula (I) preferred R⁸ is selected from the side chains of tryptophan and 1,2-benzisothiazolinylalanine.

In compounds of formula (I) preferred R⁹ is selected from the side chains of serine, histidine, lysine, ornithine, Dab, threonine, lysine, glycine, glutamic acid, valine, Dap, arginine, aspartic acid, and tyrosine.

In compounds of formula (I) preferred R¹⁰ is selected from the side chains of optionally substituted tryptophan, benzisothiazolylalanine, 1-napththylalanine, methionine.

In compounds of formula (I) preferred R¹¹ is selected from the side chains of norleucine, leucine, asparagine, phenylalanine, methionine, ethoxymethane, alanine, tryptophan, isoleucine, phenylpropane, glutamic acid, hexane, and heptane.

In compounds of formula (I) where R¹² is not part of a ring, preferred R¹² is selected from the side chains of norleucine, alanine, ethoxymethane, methionine, serine, phenylalanine, methoxyethane, leucine, tryptophan, isoleucine, glutamic acid, hexane, heptane, and glycine.

In compounds of formula (I) preferred R¹³ is selected from the side chains of arginine, ornithine, alanine, Dap, Dab, leucine, aspartic acid, glutamic acid, serine, lysine, threonine, cyclopropylmethane, glycine, valine, isoleucine, histidine, and 2-aminobutane.

In another embodiment, the present disclosure provides a compound selected from the exemplified examples within the scope of the first aspect, or a pharmaceutically acceptable salt, tautomer or stereoisomer thereof.

In another embodiment, there is provided a compound selected from any subset list of compounds within the scope of the first aspect.

In another embodiment, there is provided a compound or a pharmaceutically acceptable salt thereof which is

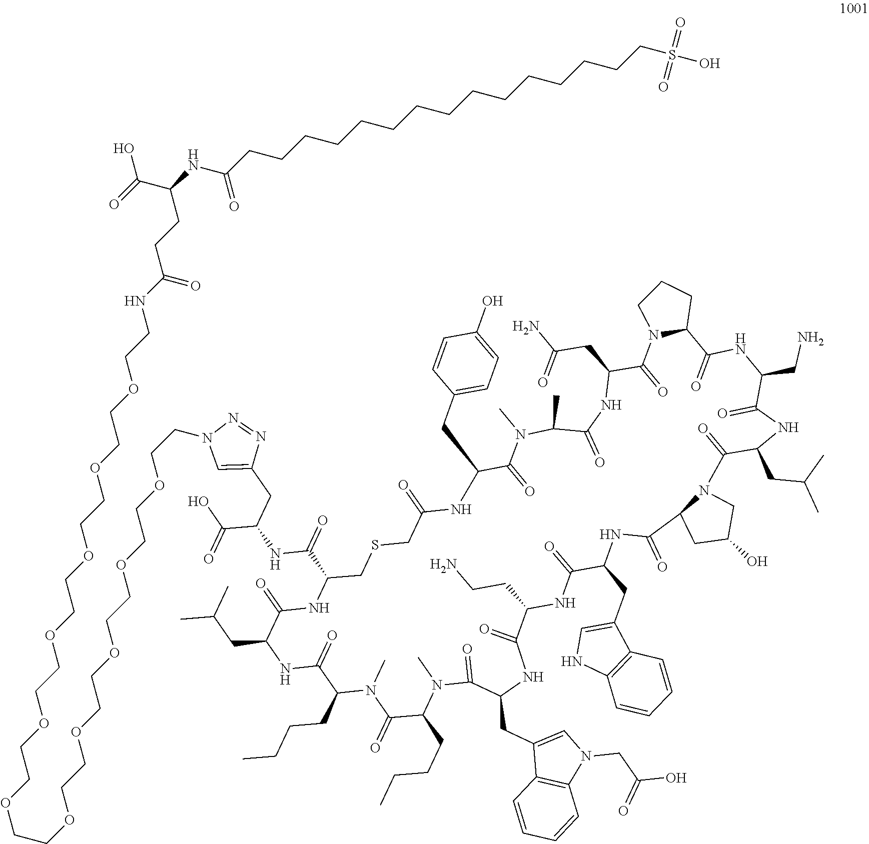 [SMILES: CCCC[C@H]1C(=O)N(C)[C@@H](CCCC)C(=O)N[C@@H](CC(C)C)C(=O)N[C@H](C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

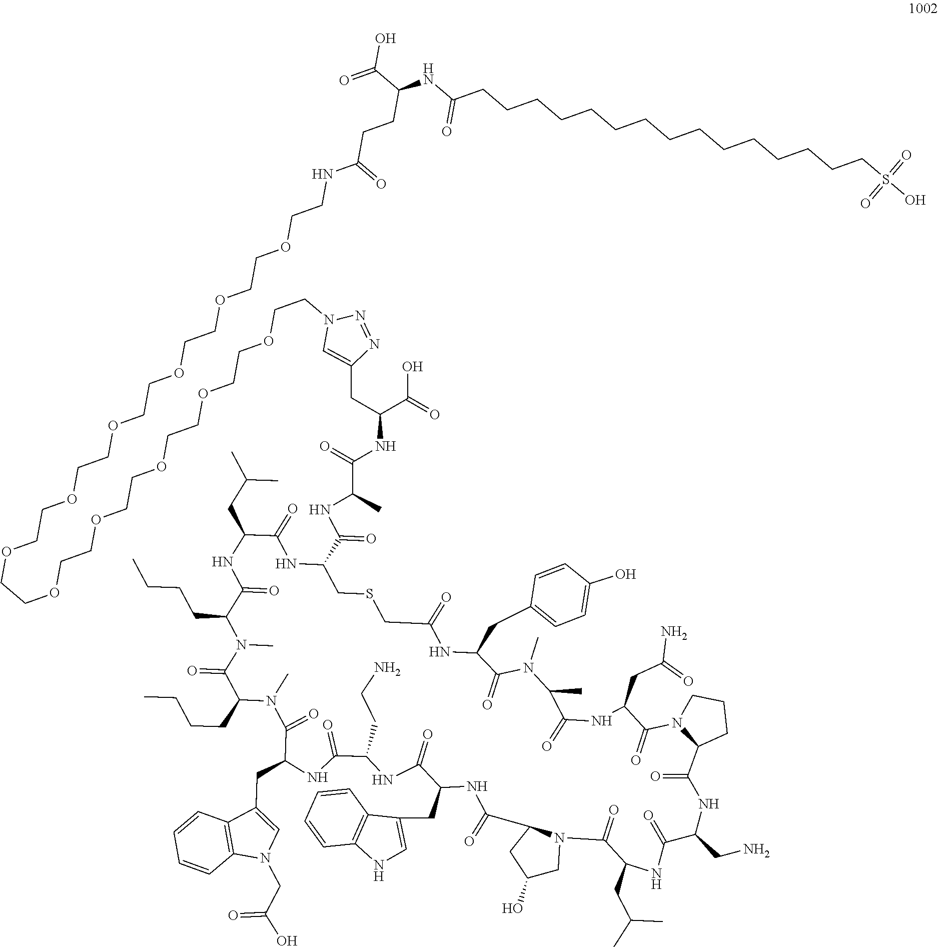 [SMILES: CCCC[C@H]1C(=O)N(C)[C@@H](CCCC)C(=O)N[C@@H](CC(C)C)C(=O)N[C@H](C(=O)N[C@H](C)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

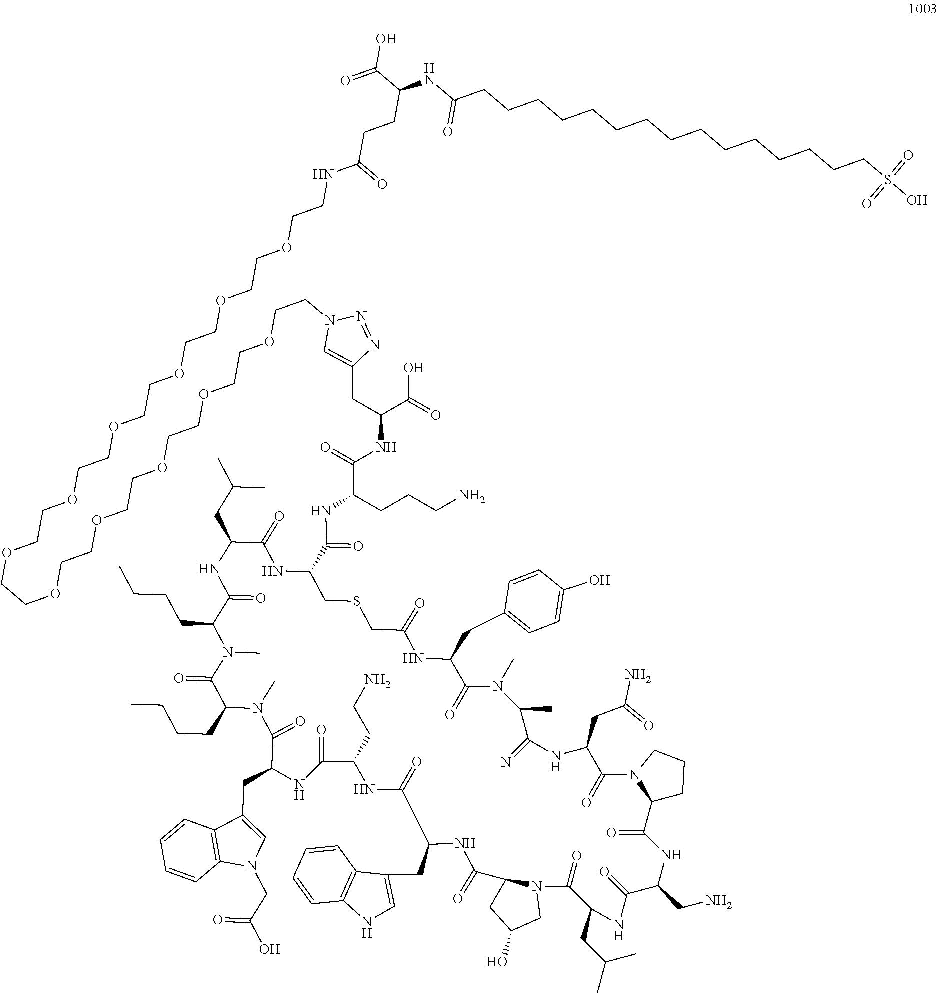 [SMILES: CCCC[C@H]1C(=O)N(C)[C@@H](CCCC)C(=O)N[C@@H](CC(C)C)C(=O)N[C@H](C(=O)N[C@@H](CCCN)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=N)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

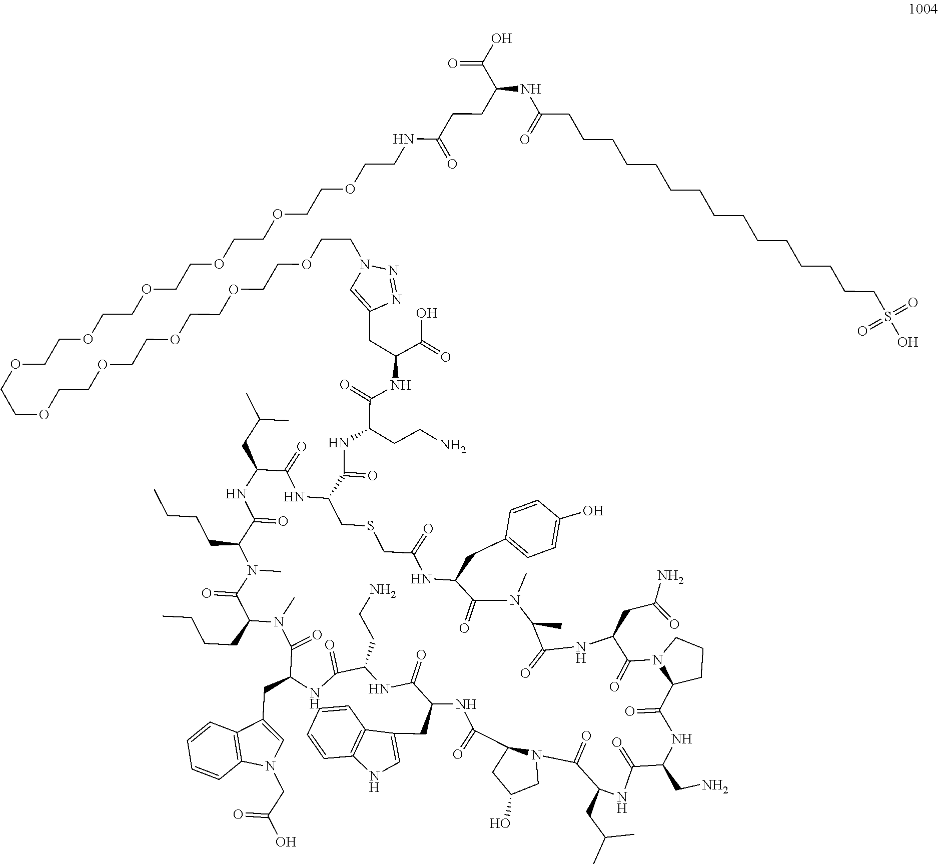 [SMILES: CCCC[C@H]1C(=O)N(C)[C@@H](CCCC)C(=O)N[C@@H](CC(C)C)C(=O)N[C@H](C(=O)N[C@@H](CCN)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

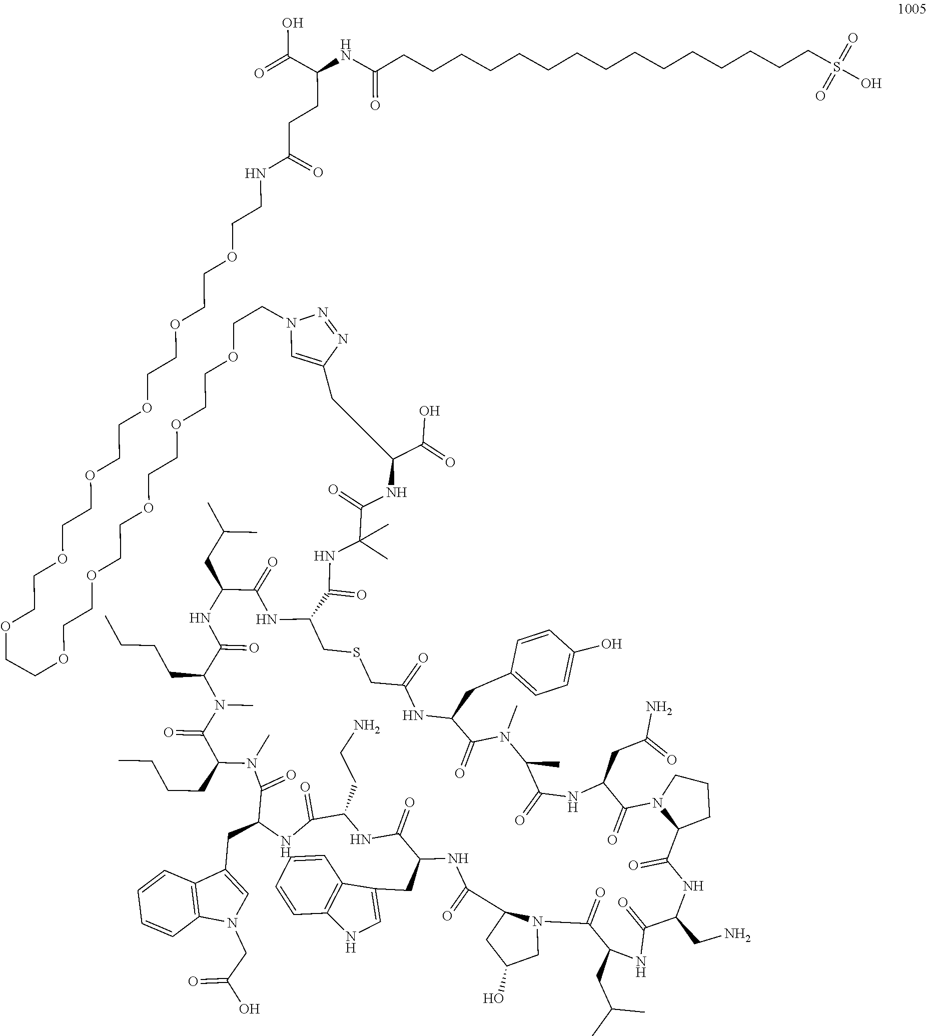 [SMILES: CCCC[C@H]1C(=O)N(C)[C@@H](CCCC)C(=O)N[C@@H](CC(C)C)C(=O)N[C@H](C(=O)NC(C)(C)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

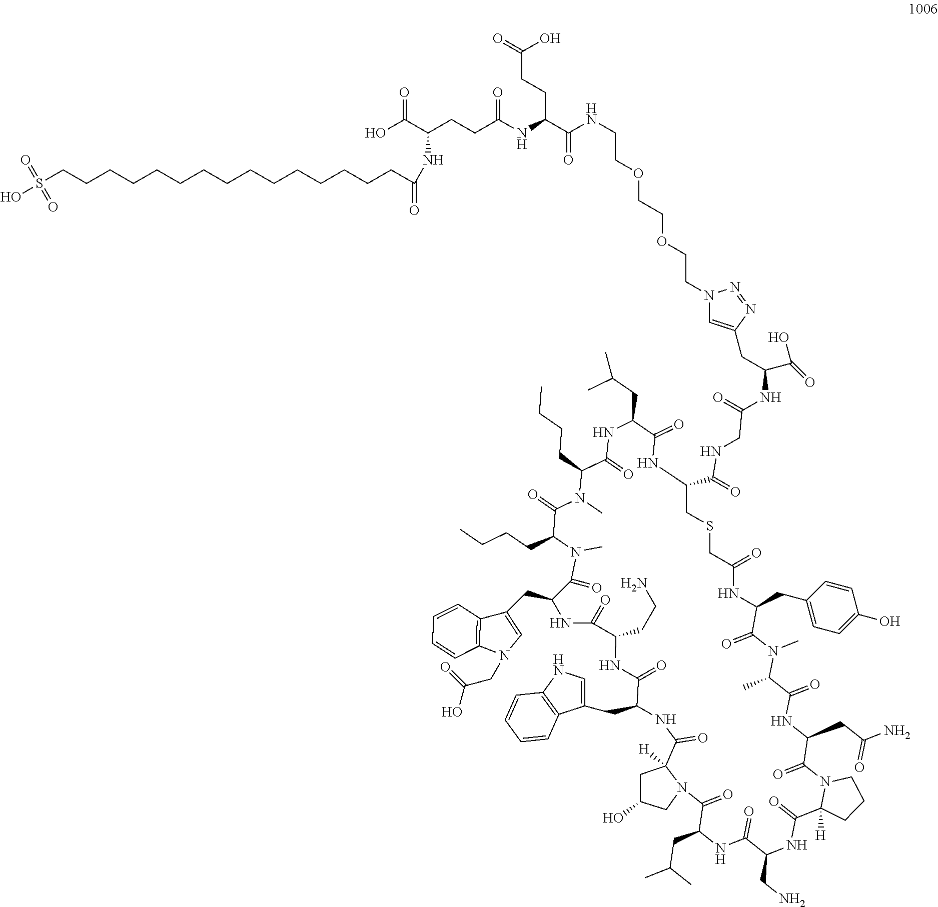 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCNC(=O)[C@H](CCC(=O)O)NC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

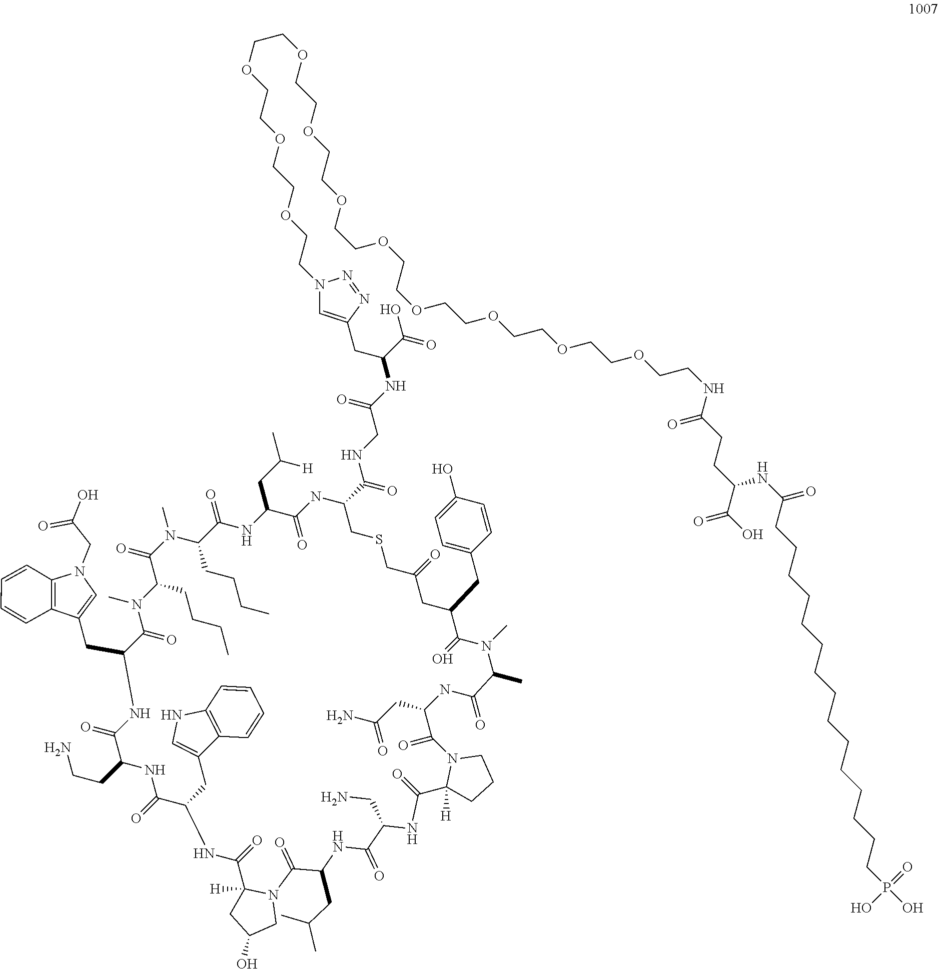 [SMILES: CCCC[C@H]1C(=O)N(C)[C@@H](CCCC)C(=O)N[C@@H](CCC)C(=O)N[C@H](C(=O)NCC(=O)N[C@@H](Cc2cn(CCOCCOCCOCCOCCOCCOCCOCCOCCOCCOCCOCCNC(=O)CC[C@H](NC(=O)CCCCCCCCCCCCCCCP(=O)(O)O)C(=O)O)nn2)C(=O)O)CSCC(=O)C[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

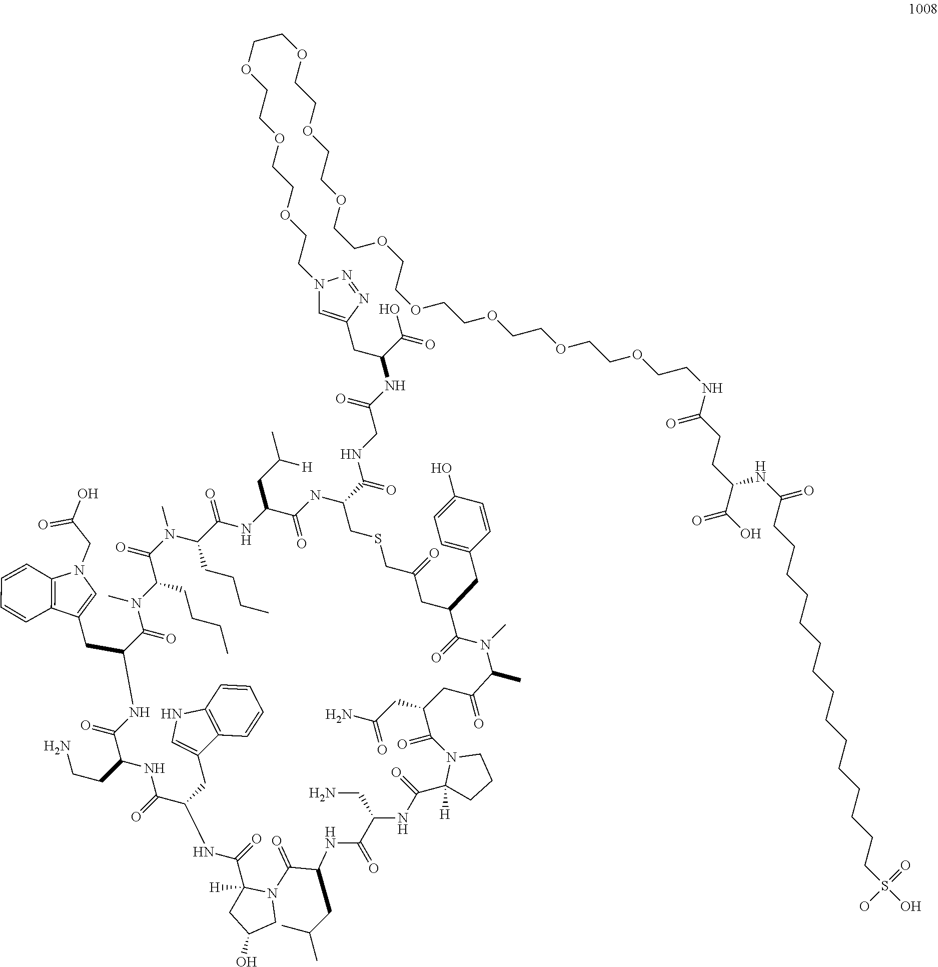

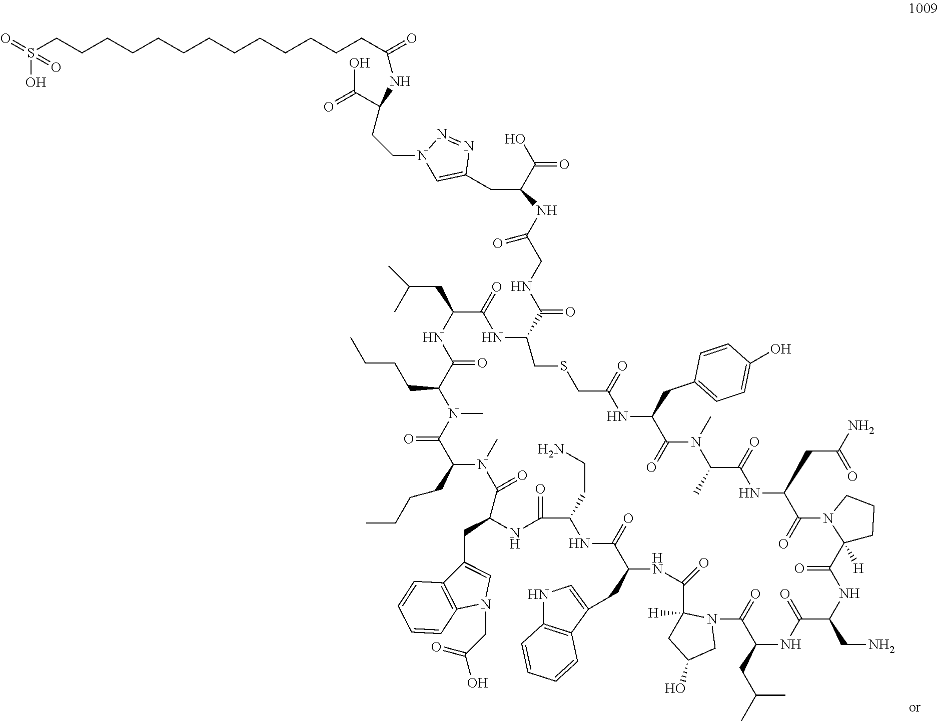 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CC[C@H](NC(=O)CCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

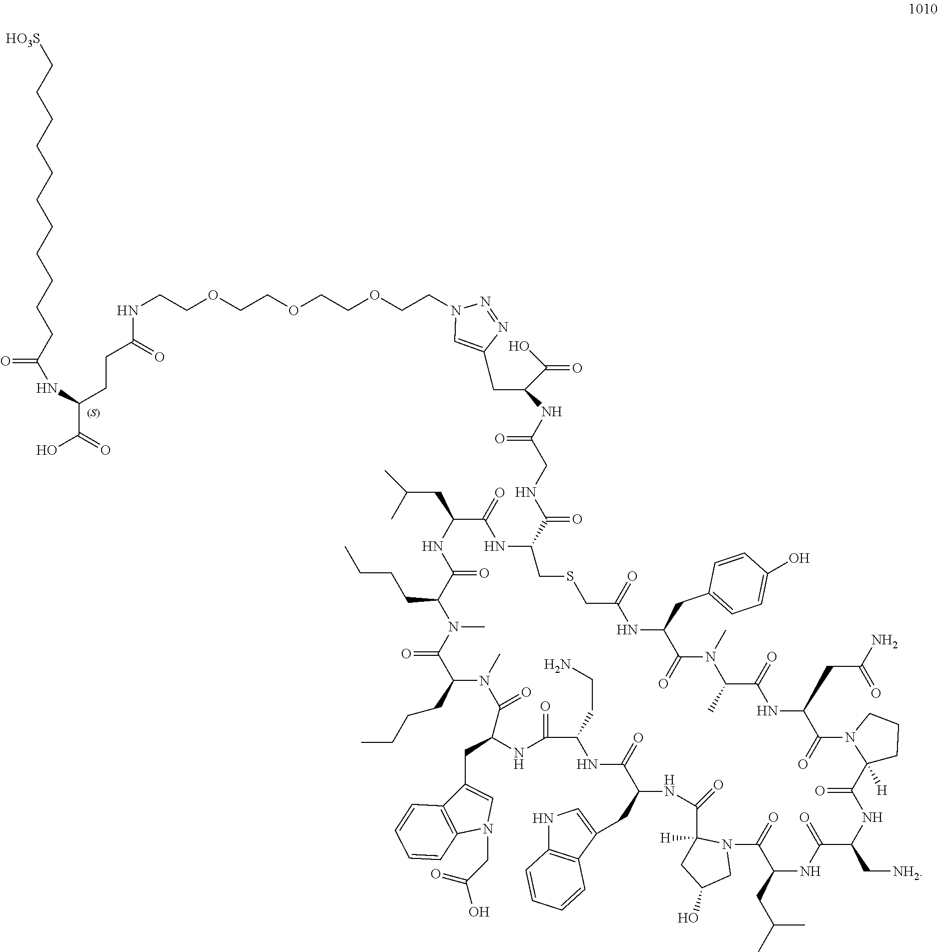

In a another aspect, the present disclosure provides a method of enhancing, stimulating, and/or increasing an immune response in a subject in need thereof, wherein the method comprises administering to the subject a therapeutically effective amount of a compound of formula (I) or formula (II), or a pharmaceutically acceptable salt thereof.

In a another aspect, the present disclosure provides a method of blocking the interaction of PD-L1 with PD-1 and/or CD80 in a subject, wherein the method comprises administering to the subject a therapeutically effective amount of a compound of formula (I) or formula (II) or a pharmaceutically acceptable salt thereof.

In another aspect, the present disclosure provides a method of enhancing, stimulating, and/or increasing an immune response in a subject in need thereof, said method comprising administering to the subject a therapeutically effective amount of a compound of formula (I) or formula (II), or a pharmaceutically acceptable salt thereof. In a first embodiment of the second aspect the method further comprises administering an additional agent prior to, after, or simultaneously with the compound of formula (I), compound of formula (I)), or a pharmaceutically acceptable salt thereof. In a second embodiment the additional agent is selected from an antimicrobial agent, an antiviral agent, a cytotoxic agent, a TLR7 agonist, a TLR8 agonist, an HDAC inhibitor, a STING, NLRP3 or DGK agent, and an immune response modifier.

In a another aspect, the present disclosure provides a method of inhibiting growth, proliferation, or metastasis of cancer cells in a subject in need thereof, said method comprising administering to the subject a therapeutically effective amount a compound of formula (I) or formula (II), or a pharmaceutically acceptable salt thereof. In a first embodiment of the third aspect the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

In a another aspect, the present disclosure provides a method of treating an infectious disease in a subject in need thereof, the method comprising administering to the subject a therapeutically effective amount of a compound of formula (I) or formula (II), or a pharmaceutically acceptable salt thereof. In a first embodiment of the fourth aspect the infectious disease is caused by a virus. In a second embodiment the virus is selected from HIV, Hepatitis A, Hepatitis B, Hepatitis C, herpes viruses, and influenza.

In a another aspect, the present disclosure provides a method of treating septic shock in a subject in need thereof, the method comprising administering to the subject a therapeutically effective amount of a compound of formula (I) or formula (II), or a pharmaceutically acceptable salt thereof.

In a another aspect, the present disclosure provides a method of blocking the interaction of PD-L1 with PD-1 and/or CD80 in a subject, said method comprising administering to the subject a therapeutically effective amount of a compound of formula (I) or formula (II), or a pharmaceutically acceptable salt thereof.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₄ alkyl” denotes straight and branched chain alkyl groups with one to four carbon atoms.

The term “cycloalkyl,” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “hydroxyalkyl” includes both branched and straight-chain saturated alkyl groups substituted with one or more hydroxyl groups. For example, “hydroxyalkyl” includes —CH₂OH, —CH₂CH₂OH, and Cu-4 hydroxyalkyl.

The term “aryl” as used herein, refers to a group of atoms derived from a molecule containing aromatic ring(s) by removing one hydrogen that is bonded to the aromatic ring(s). Representative examples of aryl groups include, but are not limited to, phenyl and naphthyl. The aryl ring may be unsubstituted or may contain one or more substituents as valence allows.

The terms “halo” and “halogen”, as used herein, refer to F, Cl, Br, or I.

The aromatic rings of the invention contain 0-3 hetero atoms selected may include from —N—, —S— and —O—. They also include heteroaryl groups as defined below.

The term “heteroaryl” refers to substituted and unsubstituted aromatic 5- or 6-membered monocyclic groups and 9- or 10-membered bicyclic groups that have at least one heteroatom (0, S or N) in at least one of the rings, said heteroatom-containing ring preferably having 1, 2, or 3 heteroatoms independently selected from O, S, and/or N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic group are aromatic and may contain only carbon atoms. The nitrogen and sulfur atoms may optionally be oxidized and the nitrogen atoms may optionally be quaternized. Bicyclic heteroaryl groups must include only aromatic rings. The heteroaryl group may be attached at any available nitrogen or carbon atom of any ring. The heteroaryl ring system may be unsubstituted or may contain one or more substituents.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, and pyrrolopyridyl.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

An “adverse event” or “AE” as used herein is any unfavorable and generally unintended, even undesirable, sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

As used herein, “hyperproliferative disease” refers to conditions wherein cell growth is increased over normal levels. For example, hyperproliferative diseases or disorders include malignant diseases (e.g., esophageal cancer, colon cancer, biliary cancer) and non-malignant diseases (e.g., atherosclerosis, benign hyperplasia, and benign prostatic hypertrophy).

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Programmed Death Ligand 1”, “Programmed Cell Death Ligand 1”, “PD-L1”, “PDL1”, “hPD-L1”, “hPD-LI”, and “B7-H1” are used interchangeably, and include variants, isoforms, species homologs of human PD-L1, and analogs having at least one common epitope with PD-L1. The complete PD-L1 sequence can be found under GENBANK® Accession No. NP_054862.

The terms “Programmed Death 1”, “Programmed Cell Death 1”, “Protein PD-1”, “PD-1”, “PDi”, “hPD-1” and “hPD-1” are used interchangeably, and include variants, isoforms, species homologs of human PD-1, and analogs having at least one common epitope with PD-1. The complete PD-1 sequence can be found under GENBANK® Accession No. U64863.

The term “treating” refers to inhibiting the disease, disorder, or condition, i.e., arresting its development; and (iii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

Those of ordinary skill in the art are aware that an amino acid includes a compound represented by the general structure:

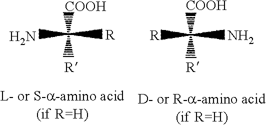

where R and R′ are as discussed herein. Unless otherwise indicated, the term “amino acid” as employed herein, alone or as part of another group, includes, without limitation, an amino group and a carboxyl group linked to the same carbon, referred to as “a” carbon, where R and/or R′ can be a natural or an un-natural side chain, including hydrogen. The absolute “S” configuration at the “a” carbon is commonly referred to as the “L” or “natural” configuration. In the case where both the “R” and the “R′” (prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which possess the ability to inhibit the interaction between PD-1 and PD-L1 and/or CD80 and PD-L1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Certain compounds of the present disclosure can exist as tautomers, which are compounds produced by the phenomenon where a proton of a molecule shifts to a different atom within that molecule. The term “tautomer” also refers to one of two or more structural isomers that exist in equilibrium and are readily converted from one isomer to another. All tautomers of the compounds described herein are included within the present disclosure.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Administration of a therapeutic agent described herein includes, without limitation, administration of a therapeutically effective amount of therapeutic agent. The term “therapeutically effective amount” as used herein refers, without limitation, to an amount of a therapeutic agent to treat a condition treatable by administration of a composition comprising the PD-1/PD-L1 binding inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration. Thus, it is not useful to specify an exact effective amount in advance.

In another aspect, the disclosure pertains to methods of inhibiting growth of tumor cells in a subject using the macrocyclic compounds of the present disclosure. As demonstrated herein, the compounds of the present disclosure are capable of binding to PD-L1, disrupting the interaction between PD-L1 and PD-1, competing with the binding of PD-L1 with anti-PD-1 monoclonal antibodies that are known to block the interaction with PD-1, enhancing CMV-specific T cell IFNγ secretion, and enhancing HIV-specific T cell IFNγ secretion. As a result, the compounds of the present disclosure are useful for modifying an immune response, treating diseases such as cancer or infectious disease, stimulating a protective autoimmune response or to stimulate antigen-specific immune responses (e.g., by co-administration of PD-L1 blocking compounds with an antigen of interest).

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents. For example, the combination therapy can include a macrocyclic compound combined with at least one other anti-inflammatory or immunosuppressant agent. Examples of therapeutic agents that can be used in combination therapy are described in greater detail below in the section on uses of the compounds of the disclosure.

As used herein, “pharmaceutically acceptable carrier” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

A pharmaceutical composition of the disclosure also can include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure can be administered via one or more routes of administration using one or more of a variety of methods known in the art. As will be appreciated by the skilled artisan, the route and/or mode of administration will vary depending upon the desired results. In some embodiments, the routes of administration for macrocyclic compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

An emulsion of at least one compound of Formula (I) and/or at least one pharmaceutically acceptable salt thereof can, for example, be prepared as an oil-in-water emulsion. The oily phase of the emulsions comprising the compounds of Formula (I) can be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase can comprise merely an emulsifier, it can comprise a mixture of at least none emulsifier with a fat or an oil or with both a fat and an oil. Suitable emulsifying agents include, but are not limited to, for example, naturally-occurring phosphatides, e.g., soy bean lecithin, esters or partial esters derived from fatty acids and hexitol anhydrides, such as, for example sorbitan monoleate, and condensation products of partial esters with ethylene oxide, such as, for example, polyoxyethylene sorbitan monooleate. In some embodiments, a hydrophilic emulsifier is included together with a lipophilic emulsifier which acts as a stabilizer. It is also sometimes desirable to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceral disterate alone or with a wax, or other materials well known in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one embodiment, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the disclosure can be formulated to ensure proper distribution in vivo. For example, the blood-brain barrier (BBB) excludes many highly hydrophilic compounds. To ensure that therapeutic compounds of the disclosure cross the BBB (if desired), they can be formulated, for example, in liposomes. For methods of manufacturing liposomes, see, e.g., U.S. Pat. Nos. 4,522,811, 5,374,548, and 5,399,331. The liposomes can comprise one or more moieties which are selectively transported into specific cells or organs, thus enhance targeted drug delivery (see, e.g., Ranade, V. V., J. Clin. Pharmacol., 29:685 (1989)). Exemplary targeting moieties include folate or biotin (see, e.g., U.S. Pat. No. 5,416,016 to Low et al.); mannosides (Umezawa et al., Biochem. Biophys. Res. Commun., 153:1038 (1988)); macrocyclic compounds (Bloeman, P. G. et al., FEBS Lett., 357:140 (1995); Owais, M. et al., Antimicrob. Agents Chemother., 39:180 (1995)); surfactant protein A receptor (Briscoe et al., Am. J. Physiol., 1233:134 (1995)); p 120 (Schreier et al., J. Biol. Chem., 269:9090 (1994)); see also Keinanen, K. et al., FEBS Lett., 346:123 (1994); Killion, J. J. et al., Immunomethods 4:273 (1994).

### 1. Peptide Synthesis

The macrocyclic peptides of the present disclosure can be produced by methods known in the art, such as they can be synthesized chemically, recombinantly in a cell free system, recombinantly within a cell or can be isolated from a biological source. Chemical synthesis of a macrocyclic peptide of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic peptides and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-Cl-HOBt or HOAt active esters produced from DIC/HOBt, HBTU/HOBt, BOP, PyBOP, or from DIC/6-C1-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the peptide analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown below.

### Examples of Orthogonally Protected Amino Acids Used in Solid Phase Synthesis

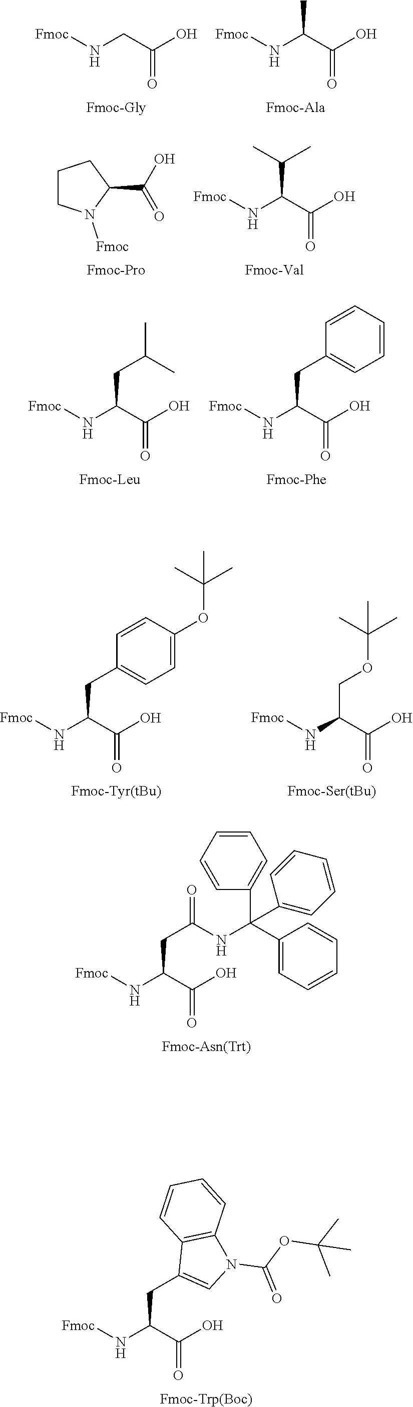

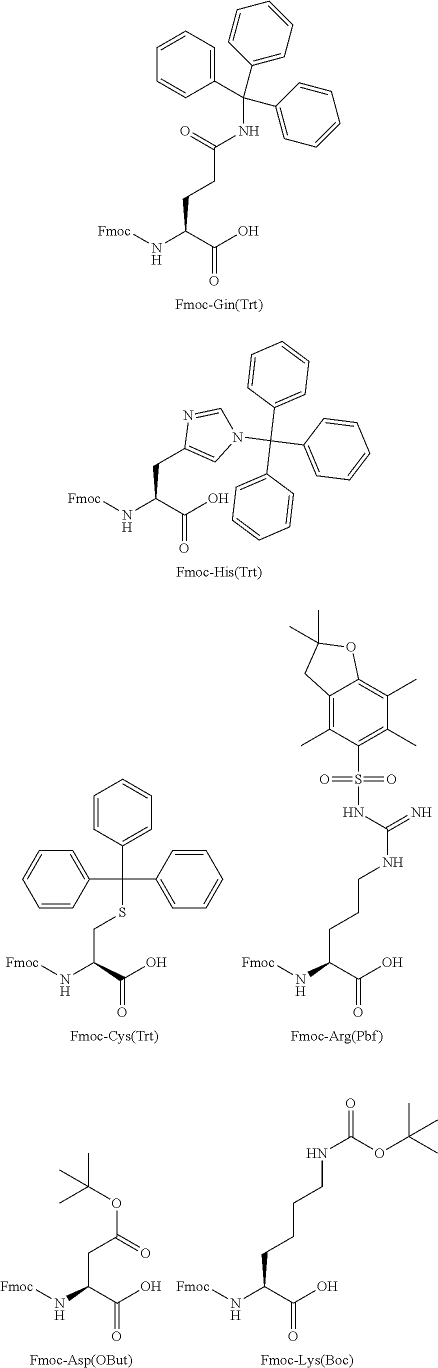

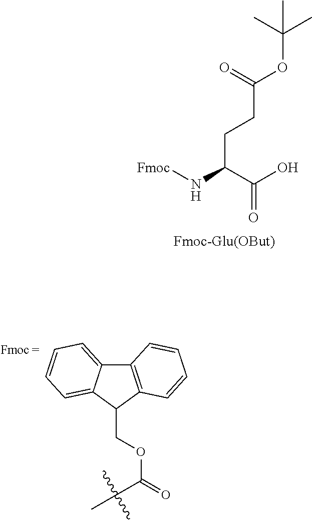

The peptidyl-resin precursors for their respective peptides may be cleaved and deprotected using any standard procedure (see, for example, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 hrs at room temperature. The spent resin is then filtered off and the TFA solution was cooled and Et₂O solution was added. The precipitates were collected by centrifuging and decanting the ether layer (3×). The resulting crude peptide is either redissolved directly into DMF or DMSO or CH₃CN/H₂O for purification by preparative HPLC or used directly in the next step.

Peptides with the desired purity can be obtained by purification using preparative HPLC, for example, on a Waters Model 4000 or a Shimadzu Model LC-8A liquid chromatography. The solution of crude peptide is injected into a YMC S5 ODS (20×100 mm) column and eluted with a linear gradient of MeCN in water, both buffered with 0.1% TFA, using a flow rate of 14-20 mL/min with effluent monitoring by UV absorbance at 217 or 220 nm. The structures of the purified peptides can be confirmed by electro-spray MS analysis.

List of non-naturally occurring amino acids referred to herein is provided below.

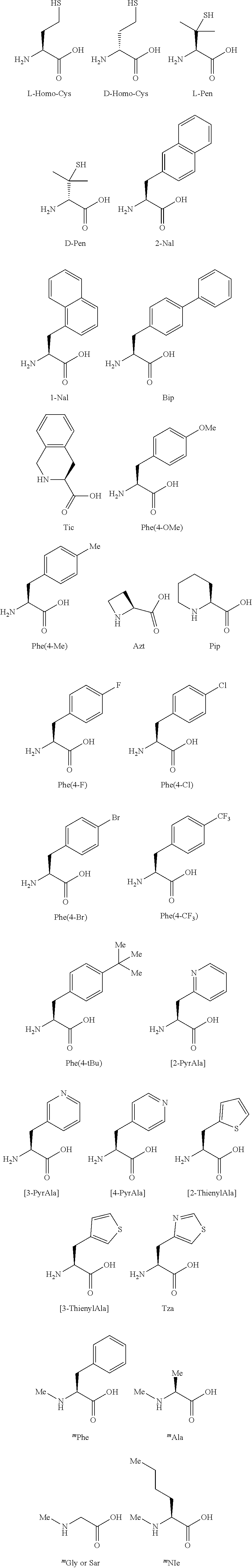

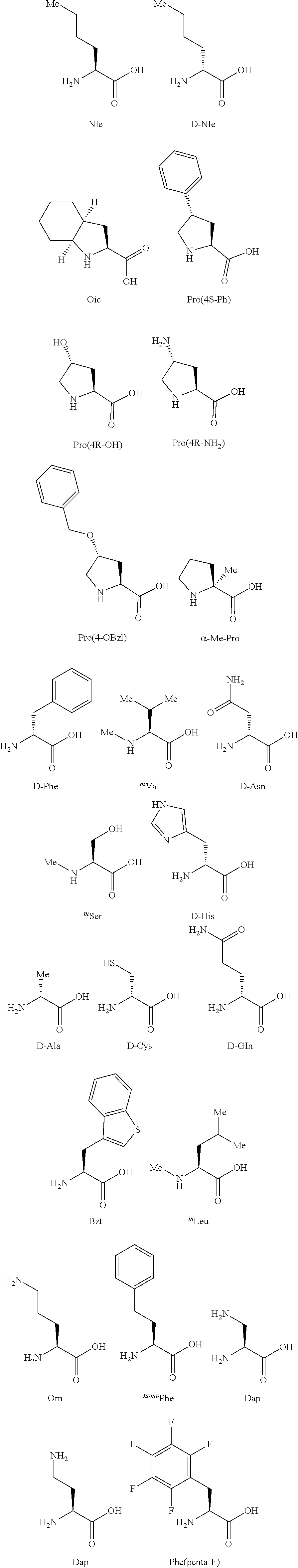

The following abbreviations are employed in the Examples and elsewhere herein:

| | |
|---|---|
| Ph | phenyl |
| Bn | benzyl |
| i-Bu | iso-butyl |
| i-Pr | iso-propyl |
| Me | methyl |
| Et | ethyl |
| Pr | n-propyl |
| Bu | n-butyl |
| t-Bu | tert-butyl |
| Trt | trityl |
| TMS | trimethylsilyl |
| TIS | triisopropylsilane |
| Et₂O | diethyl ether |
| HOAc or AcOH | acetic acid |
| MeCN or AcCN | acetonitrile |
| DMF | N,N-dimethylformamide |
| EtOAc | ethyl acetate |
| THF | tetrahydrofuran |
| TFA | trifluoroacetic acid |
| TFE | α,α,α-trifluoroethanol |
| Et₂NH | diethylamine |
| NMM | N-methylmorpholine |
| NMP | N-methylpyrrolidone |
| DCM | dichloromethane |
| TEA | triethylamine |
| min. | minute(s) |
| h or hr | hour(s) |
| L | liter |
| mL or ml | milliliter |
| μL | microliter |
| g | gram(s) |
| mg | milligram(s) |
| mol | mole(s) |
| mmol | millimole(s) |
| meq | milliequivalent |
| rt or RT | room temperature |
| sat or sat'd | saturated |
| aq. | aqueous |
| mp | melting point |
| BOP reagent | benzotriazol-1-yloxy-tris-dimethylamino- |
| | phosphonium hexafluorophosphate |
| | (Castro's reagent) |
| PyBOP reagent | benzotriazol-1-yloxy-tripyrrolidino |
| | phosphonium hexafluorophosphate |
| HBTU | 2-(1H-Benzotriazol-1-yl)-1,1,3,3- |
| | tetramethyluronim hexafluorophosphate |
| HATU | O-(7-Azabenzotriazol-1-yl)-1,1,3,3- |
| | tetramethyluronim hexafluorophosphate |
| HCTU | 2-(6-Chloro-1-H-benzotriazol-1-yl)- |
| | 1,1,3,3-tetramethyluronium |
| | hexafluorophosphate |
| T3P | 2,4,6-tripropyl-1,3,5,2,4,6- |
| | trioxatriphosphorinane-2,4,6-trioxide |
| DMAP | 4-(dimethylamino)pyridine |
| DIEA | diisopropylethylamine |
| Fmoc or FMOC | fluorenylmethyloxycarbonyl |
| Boc or BOC | tert-butyloxycarbonyl |
| HOBT or HOBT•H₂O | 1-hydroxybenzotriazole hydrate |
| Cl-HOBt | 6-Chloro-benzotriazole |
| HOAT | 1-hydroxy-7-azabenzotriazole |
| HPLC | high performance liquid chromatography |
| LC/MS | high performance liquid |
| | chromatography/mass spectrometry |
| MS or Mass Spec | mass spectrometry |
| NMR | nuclear magnetic resonance |
| Sc or SC or SQ | sub-cutaneous |
| IP or ip | intra-peritoneal |

### Example 0001—Solid Phase Peptide Synthesis and Cyclization of Peptides

The procedures described in this example, either in whole or in part where noted, were used to synthesize the macrocyclic peptides shown in Table 1

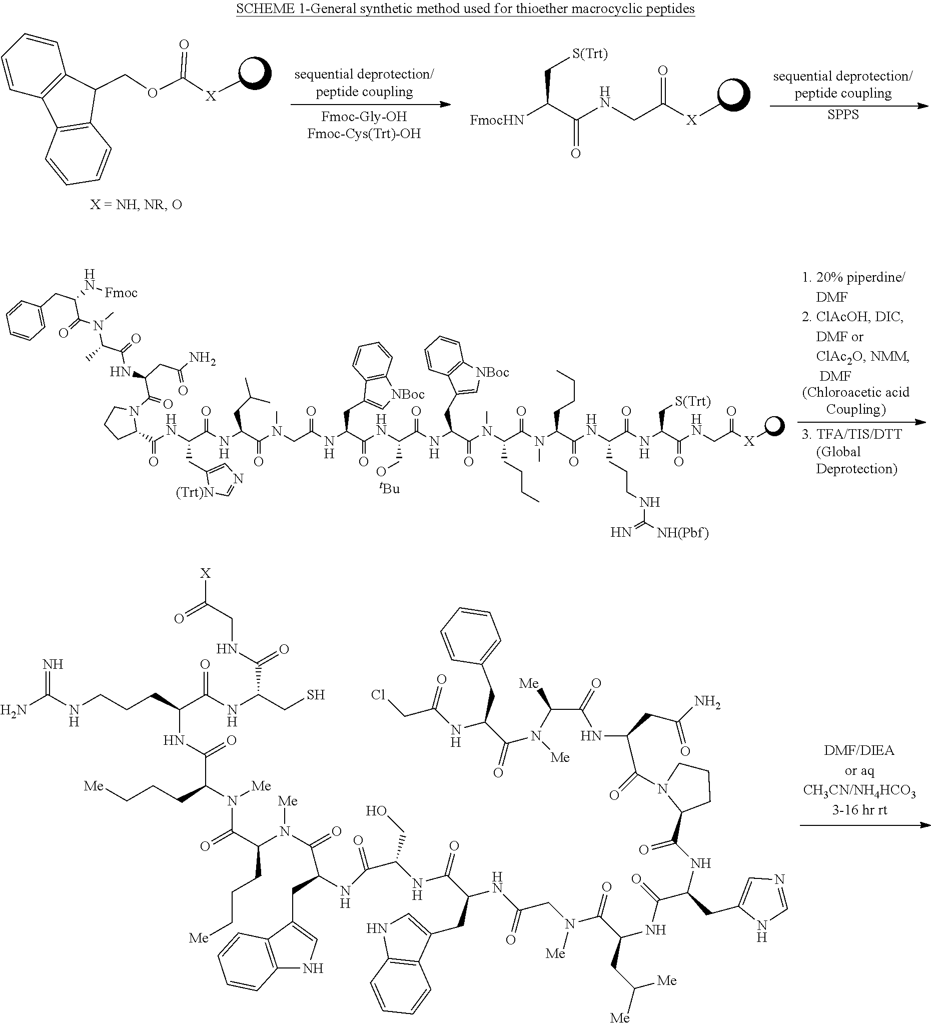

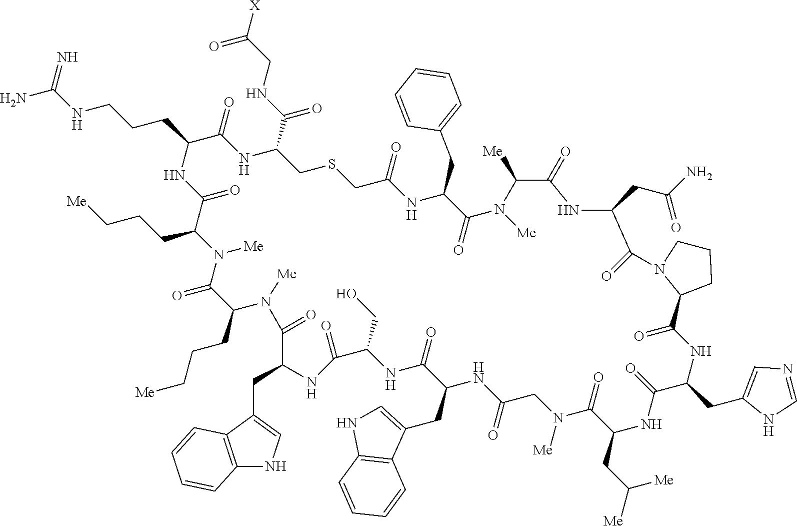

### General Protocol for Solid-Phase Peptide Synthesis (SPPS) and Macrocyclization.

On a Symphony Peptide Synthesizer (Protein Technology Inc. Tucson, AZ), Prelude Peptide Synthesizer (Protein Technology Inc. Tucson, AZ), or Symphony X Peptide Synthesizer (Protein Technology Inc. Tucson, AZ), Chlorotrityl resin preloaded with Fmoc-Pra-OH (0.100 mmol) was swelled with CH₂Cl₂ then DMF when mixing under a gentle stream of N₂. The solvent was drained and the following method was used to couple the first amino acid: the Fmoc group was removed from the resin-supported building block by washing the resin twice with a solution of 20% piperidine in DMF when mixing with a gentle stream of N₂ every 30 seconds. The resin was washed five to six times with DMF. Fmoc-Gly-OH (0.2 M solution in DMF) was then added, followed by coupling activator (i.e., HATU (Chem-Impex Int'l, 0.4 M solution in DMF) and base (i.e., N-methyl morpholine (Aldrich, 0.8 M in DMF). The reaction mixture was agitated by a gentle stream of nitrogen for 1-2 h. The reagents were drained from the reaction vessel, and the resin was washed five to six times with DMF. The resulting resin-supported Fmoc-protected dipeptide was then sequentially deprotected and coupled with third amino acid and so forth in an iterative fashion to give the desired resin-supported product.

The Fmoc group was removed from the N-terminus by washing the resin twice with a solution of 20% piperidine in DMF by a gentle stream of nitrogen. The resin was washed with DMF (5-6×). To the peptide-resin was treated with chloroacetic anhydride (0.2 M in DMF) followed by NMM (0.8 M in DMF). This reaction was repeated. After draining all the reagents and solvents, the resin was washed with DMF and DCM, and then dried.

LCMS analysis was performed on a peptide aliquot, which was cleaved from the resin (analytical amount was treated with a small amount of TFA/TIS/DTT (96:4:1) solution at room temperature to confirm the formation of the desired linear sequence.

The peptide was globally deprotected and cleaved from the resin upon treatment with a TFA/TIS/DTT (96:4:1) solution for 1.5 h. The resin was removed by filtration, washed with a small amount of cleavage cocktail, and the combined filtrates were added to cold Et₂O. The solution was chilled at 0° C. in order to effect the peptide to precipitate out of solution. The slurry was centrifuged to pellet the solids and the supernatant was decanted. Fresh Et₂O was added and the process was repeated three times to wash the solids. To the air-dried solids was added a solution of DIEA/DMF (1-3 mL of DIEA in 40-45 mL of DMF) or 0.1 M NH₄HCO₃/acetonitrile (from 1/1 to 3/1 (v/v)) so that the pH of the solution was greater than 8. The solution was stirred for 16-72 h and monitored by LCMS. The reaction solution was purified by preparative reverse phase HPLC to obtain the desired product.

### General Analytical Protocols and Synthesis Methods

### Analytical Data:

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “m/z” unit designation. Compounds with exact masses greater than 1000 were often detected as double-charged or triple-charged ions.

The crude material was purified via preparative LC/MS. Fractions containing the desired product were combined and dried via centrifugal evaporation.

### Analytical LC/MS Condition A:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LC/MS Condition B:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

### Analytical LC/MS Condition C.

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 70° C.; Gradient: 0-100% B over 3 minutes, then a 2.0-minute hold at 100% B; Flow: 0.75 mL/min; Detection: UV at 220 nm.

### Analytical LC/MS Condition D:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 70° C.; Gradient: 0-100% B over 3 minutes, then a 2.0-minute hold at 100% B; Flow: 0.75 mL/min; Detection: UV at 220 nm.

### Analytical LC/MS Condition E:

Column: Kinetex XB C18, 3.0×75 mm, 2.6-μm particles; Mobile Phase A: 10 mM ammonium formate in water:acetonitrile (98:2); Mobile Phase B: 10 mM ammonium formate in Water:acetonitrile (02:98); Gradient: 20-100% B over 4 minutes, then a 0.6-minute hold at 100% B; Flow: 1.0 mL/min; Detection: UV at 254 nm.

### Analytical LC/MS Condition F.

Column: Ascentis Express C18, 2.1×50 mm, 2.7-μm particles; Mobile Phase A: 10 mM ammonium acetate in water:acetonitrile (95:5); Mobile Phase B: 10 mM ammonium acetate in Water:acetonitrile (05:95), Temperature: 50° C.; Gradient: 0-100% B over 3 minutes; Flow: 1.0 mL/min; Detection: UV at 220 nm.

### Analytical LC/MS Condition G:

Column: X Bridge C18, 4.6×50 mm, 5-μm particles; Mobile Phase A: 0.1% TFA in water; Mobile Phase B: acetonitrile, Temperature: 35° C.; Gradient: 5-95% B over 4 minutes; Flow: 4.0 mL/min; Detection: UV at 220 nm.

### Analytical LC/MS Condition H.

Column: X Bridge C18, 4.6×50 mm, 5-μm particles; Mobile Phase A: 10 mM NH₄OAc; Mobile Phase B: methanol, Temperature: 35° C.; Gradient: 5-95% B over 4 minutes; Flow: 4.0 mL/min; Detection: UV at 220 nm.

### Analytical LC/MS Condition I.

Column: X Bridge C18, 4.6×50 mm, 5-μm particles; Mobile Phase A: 10 mM NH₄OAc; Mobile Phase B: acetonitrile, Temperature: 35° C.; Gradient: 5-95% B over 4 minutes; Flow: 4.0 mL/min; Detection: UV at 220 nm.

### Analytical LC/MS Condition J.

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 70° C.; Gradient: 0-100% B over 1.5 minutes, then a 2.0-minute hold at 100% B; Flow: 0.75 mL/min; Detection: UV at 254 nm.

### Analytical LC/MS Condition K.

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 1.0 minutes, then at 1.0-1.5 minute hold at 98% B; Flow: 0.80 mL/min; Detection: UV at 220 nm.

### Analytical LC/MS Condition L:

Column: Waters Acquity UPLC BEH C18, 2.1×50 mm, 1.7-μm particles; Buffer: 10 mM Ammonium Acetate. Mobile Phase A: buffer” CH3CN (95/5); Mobile Phase B: Mobile Phase B:Buffer:ACN(5:95); Temperature: 50° C.; Gradient: 20-98% B over 2.0 minutes, then at 0.2 minute hold at 100% B; Flow: 0.70 mL/min; Detection: UV at 220 nm.

### Analytical LC/MS Condition M.

Column: Waters Acquity UPLC BEH C18, 3.0×50 mm, 1.7-μm particles; Mobile Phase A: 95% water and 5% water with 0.1% trifluoroacetic acid; Mobile Phase B: 95% acetonitrile and 5% water with 0.1% trifluoroacetic acid; Temperature: 50° C.; Gradient: 20-100% B over 2.0 minutes, then at 2.0-2.3 minute hold at 100% B; Flow: 0.7 mL/min; Detection: UV at 220 nm.

### Analytical LC/MS Condition N.

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 5.0 minutes, then at 5.0-5.5 minute hold at 98% B; Flow: 0.80 mL/min; Detection: UV at 220 nm.

### Analytical LC/MS Condition O:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 2%-98% B over 2 minutes, then a 0.5-minute hold at 98% B; Flow: 0.8 mL/min; Detection: UV at 220 nm.

### Analytical LC/MS Condition P.

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 0%-100% B over 3 minutes, then a 0.5-minute hold at 100% B; Flow: 1.0 mL/min; Detection: UV at 220 nm.

### Analytical LC/MS Condition Q:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 0%-100% B over 1 minute, then a 0.5-minute hold at 100% B; Flow: 1.0 mL/min; Detection: UV at 220 nm.

### Analytical LC/MS Condition R:

Column: Waters Acquity UPLC BEH C18, 2.1×50 mm, 1.7-μm particles; Buffer: 10 mM Ammonium Acetate. Mobile Phase A: buffer” CH3CN (95/5); Mobile Phase B: Mobile Phase B:Buffer:ACN(5:95); Temperature: 50° C.; Gradient: 0%-100% B over 1 minute, then a 0.5-minute hold at 100% B; Flow: 1.0 mL/min; Detection: UV at 220 nm.

### Analytical LC/MS Condition S:

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Gradient: 2-98% B over 1.6 minutes, then at 0.2 minute hold at 98% B; Flow: 0.80 mL/min; Detection: UV at 220 nm.

### Analytical LC/MS Condition T.

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Gradient: 2%-98% B over 2.6 minutes, then a 0.4-minute hold at 98% B; Flow: 0.8 mL/min; Detection: UV at 220 nm.

### Analytical LC/MS Condition U.

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Gradient: 30-100% B over 3 minutes, then a 0.75-minute hold at 100% B; Flow: 1.0 mL/min; Detection: UV at 220 nm.

### General Procedures:

### Prelude Method:

All manipulations were performed under automation on a Prelude peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N2 gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis.

Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Prelude Method” describe an experiment performed on a 0.100 mmol scale, where the scale is determined by the amount of Sieber or Rink or 2-chlorotrityl or PL-FMP resin. This scale corresponds to approximately 140 mg of the Sieber amide resin described above. All procedures can be scaled down or up from the 0.100 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Double-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hour, then the reaction solution was drained through the frit. The resin was washed successively two times as follows: for each wash, DMF (5.0 mL) was added through the top of the vessel and the resulting mixture was periodically agitated for 1.0 minute before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hours, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Single-Coupling Manual Addition Procedure A:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-2 mL) was added manually using a pipette from the top of the vessel while the bottom of the vessel was remain attached to the instrument, then the vessel was closed. The automatic program was resumed and HATU (0.4 M in DMF, 1.3 mL, 4 equiv) and NN (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, followed by the manual addition of HATU (2-4 equiv, same equiv as the unnatural amino acid), and then the vessel was closed. The automatic program was resumed and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 60 seconds before the solution was drained through the frit. To the reaction vessel was added bromoacetic acid (0.4 M in DMF, 2.0 mL, 16 eq), then DIC (0.4 M in DMF, 2.0 mL, 16 eq). The mixture was periodically agitated for 1 hour, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. To the reaction vessel was added the amine (0.4 M in DMF, 2.0 mL, 16 eq). The mixture was periodically agitated for 1 hour, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed twice as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed successively five times as follows: for each wash, DMF (6.0 mL) was added through the top of the vessel and the resulting mixture was periodically agitated for one minute before the solution was drained through the frit. The resin was washed successively four times as follows: for each wash, DCM (6.0 mL) was added through the top of the vessel and the resulting mixture was periodically agitated for one minute before the solution was drained through the frit. The resin was then dried with nitrogen flow for 10 minutes. The resulting resin was used directly in the next step.

### Symphony Method:

All manipulations were performed under automation on a 12-channel Symphony peptide synthesizer (Protein Technologies). Unless noted, all procedures were performed in a 25-mL polypropylene reaction vessel fitted with a bottom frit. The reaction vessel connects to the Symphony peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N2 gas through the bottom frit; the pulse lasts approximately 5 seconds and occurs every 30 seconds. Amino acid solutions were generally not used beyond two weeks from preparation. HATU solution were used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading.

PL-FMP resin: (4-Formyl-3-methoxyphenoxymethyl)polystyrene.

Fmoc-glycine-2-chlorotrityl chloride resin, 200-400 mesh, 1% DVB, 0.63 mmol/g loading.

Common amino acids used are listed below with side-chain protecting groups indicated inside parenthesis:

Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony Method” describe an experiment performed on a 0.05 mmol scale, where the scale is determined by the amount of Sieber or Rink or chlorotrityl linker or PL-FMP bound to the resin. This scale corresponds to approximately 70 mg of the Sieber resin described above. All procedures can be scaled up from the 0.05 mmol scale by adjusting the described volumes by the multiple of the scale.

Prior to the amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.

### Resin-Swelling Procedure:

To a 25-mL polypropylene solid-phase reaction vessel was added the resin (0.05 mmol). The resin was washed (swelled) as follows: to the reaction vessel was added DMF (2.0-3.0 mL, 1-2 times), upon which the mixture was periodically agitated for 10 minutes before the solvent was drained through the frit. Sometimes the resin was washed (swelled) as follows: to the reaction vessel was added CH₂Cl₂ (3-5 mL, 2 times) which the mixture was periodically agitated for 30 min and the solvent was drained through the frit. Then DMF (2.0-3.0 mL, 1-6 times), upon which the mixture was periodically agitated for 2-10 minutes before the solvent was drained through the frit.

### Single-Coupling Procedure:

To the reaction vessel containing the resin from the previous step was added DMF (2.5-3.75 mL) three times, upon which the mixture was agitated for 30 seconds before the solvent was drained through the frit each time. To the resin was added piperidine:DMF (20:80 v/v, 3.0-3.75 mL). The mixture was periodically agitated for 5.0 minutes and then the solution was drained through the frit. To the reaction vessel was added piperidine:DMF (20:80 v/v, 3.0-3.75 mL). The mixture was periodically agitated for 5.0 minutes and then the solution was drained through the frit. Sometimes the deprotection step was performed the third time. The resin was washed successively six times as follows: for each wash, DMF (2.5-3.75 mL) was added to the vessel and the resulting mixture was periodically agitated for 30 seconds before the solution was drained through the frit. To the reaction vessel was added the amino acid (0.2 M in DMF, 2.0-2.5 mL, 8-10 equiv), then HATU (0.4 M in DMF, 1.0-1.25 mL, 8-10 equiv), and finally NN (0.8 M in DMF, 1.0-1.25 mL, 20 equiv). The mixture was periodically agitated for 30-120 minutes, then the reaction solution was drained through the frit. The resin was washed successively six times as follows: for each wash, DMF (2.5-3.0 mL) was added and the resulting mixture was periodically agitated for 30 seconds before the solution was drained through the frit. The resulting resin was used directly in the next step.

### Single-Coupling Manual Addition Procedure:

To the reaction vessel containing the resin from the previous step was added DMF (3.0-3.75 mL) three times, upon which the mixture was agitated for 30 seconds before the solvent was drained through the frit each time. To the resin was added piperidine:DMF (20:80 v/v, 3.0-3.75 mL). The mixture was periodically agitated for 5.0 minutes and then the solution was drained through the frit. To the reaction vessel was added piperidine:DMF (20:80 v/v, 3.0-3.75 mL). The mixture was periodically agitated for 5.0 minutes and then the solution was drained through the frit. The mixture was periodically agitated for 5.0 minutes and then the solution was drained through the frit. The resin was washed successively six times as follows: for each wash, DMF (3.0-3.75 mL) was added to the vessel and the resulting mixture was periodically agitated for 30 seconds before the solution was drained through the frit. To the reaction vessel was added the premixed amino acid (2.0-5.0 equiv) and HATU (0.4 M in DMF, 2.0-5.0 equiv), then NMM (0.8 M in DMF, 4.0-10.0 equiv) and the molar ratio for amino acid, HATU, and NN was 1:1:2. The mixture was periodically agitated for 2-6 hours, then the reaction solution was drained through the frit. The resin was washed successively four times as follows: for each wash, DMF (3.75 mL) was added and the resulting mixture was periodically agitated for 30 seconds before the solution was drained through the frit. The resulting resin was used directly in the next step.

### Double-Coupling Procedure:

To the reaction vessel containing resin from the previous step was added DMF (2.5-3.75 mL) three times, upon which the mixture was agitated for 30 seconds before the solvent was drained through the frit each time. To the reaction vessel was added piperidine:DMF (20:80 v/v, 3.0-3.75 mL). The mixture was periodically agitated for 5 minutes and then the solution was drained through the frit. To the reaction vessel was added piperidine:DMF (20:80 v/v, 3.0-3.75 mL). The mixture was periodically agitated for 5 minutes and then the solution was drained through the frit. The resin was washed successively six times as follows: for each wash, DMF (3.0-3.75 mL) was added and the resulting mixture was periodically agitated for 30 seconds before the solution was drained through the frit. To the reaction vessel was added the amino acid (0.2 M in DMF, 2.0-2.5 mL, 8-10 equiv), then HATU (0.4 M in DMF, 1.0-1.25 mL, 10 equiv), and finally NMM (0.8 M in DMF, 1.0-1.25 mL, 16-20 equiv). The mixture was periodically agitated for 1 hour, then the reaction solution was drained through the frit. The resin was washed twice with DMF (3.0-3.75 mL) and the resulting mixture was periodically agitated for 30 seconds before the solution was drained through the frit each time. To the reaction vessel was added the amino acid (0.2 M in DMF, 2.0-2.5 mL, 8-10 equiv), then HATU (0.4 M in DMF, 1.0-1.25 mL, 8-10 equiv), and finally NMM (0.8 M in DMF, 1.0-1.25 mL, 16-20 eq). The mixture was periodically agitated for 1-2 hours, then the reaction solution was drained through the frit. The resin was successively washed six times as follows: for each wash, DMF (3.0-3.75 mL) was added and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine: DMF (20:80 v/v, 3.75 mL). The mixture was periodically agitated for 5 minutes and then the solution was drained through the frit. To the reaction vessel was added piperidine: DMF (20:80 v/v, 3.75 mL). The mixture was periodically agitated for 5 minutes and then the solution was drained through the frit. The resin was washed successively six times as follows: for each wash, DMF (3.75 mL) was added to the vessel and the resulting mixture was periodically agitated for 30 seconds before the solution was drained through the frit. To the reaction vessel was added the bromoacetic acid (0.4 M in DMF, 2.5 mL, 10 eq), then DIC (0.4 M in DMF, 2.5 mL, 10 eq). The mixture was periodically agitated for 60 mins, then the reaction solution was drained through the frit. The resin was washed successively two times as follows: for each wash, DMF (3.75 mL) was added to the vessel and the resulting mixture was periodically agitated for 30 seconds before the solution was drained through the frit. To the reaction vessel was added the amine (0.4 M in DMF, 2.5 mL, 10 eq), the mixture was periodically agitated for 60 mins, and then the reaction solution was drained through the frit. The resin was washed successively five times as follows: for each wash, DMF (3.75 mL) was added to the vessel and the resulting mixture was periodically agitated for 30 seconds before the solution was drained through the frit. The resulting resin was used directly in the next step.

### Chloroacetic Anhydride Coupling:

To the reaction vessel containing resin from the previous step was added DMF (3.0-3.75 mL) three times, upon which the mixture was agitated for 30 seconds before the solvent was drained through the frit each time. To the reaction vessel containing the resin from the previous step was added piperidine:DMF (20:80 v/v, 3.0-3.75 mL). The mixture was periodically agitated for 5 minutes and then the solution was drained through the frit. To the reaction vessel was added piperidine:DMF (20:80 v/v, 3.0-3.75 mL). The mixture was periodically agitated for 5 minutes and then the solution was drained through the frit. The resin was washed successively six times as follows: for each wash, DMF (3.0-3.75 mL) was added through the top of the vessel and the resulting mixture was periodically agitated for 30 seconds before the solution was drained through the frit. To the reaction vessel was added the chloroacetic anhydride solution (0.4 M in DMF, 3.0-3.75 mL, 30 equiv), then NMM (0.8 M in DMF, 2.5 mL, 40 equiv). The mixture was periodically agitated for 15 minutes, then the reaction solution was drained through the frit. The resin was washed once as follows: DMF (5.0-6.25 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NMM (0.8 M in DMF, 2.5 mL, 40 equiv). The mixture was periodically agitated for 15 minutes, then the reaction solution was drained through the frit. The resin was washed successively six times as follows: for each wash, DMF (2.5 mL) was added through the top of the vessel and the resulting mixture was periodically agitated for 30 seconds before the solution was drained through the frit. The resin was washed successively four times as follows: for each wash, DCM (2.5 mL) was added through the top of the vessel and the resulting mixture was periodically agitated for 30 seconds before the solution was drained through the frit. The resulting resin was dried using a nitrogen flow for 10 mins before being used directly in the next step.

### Symphony X Methods:

All manipulations were performed under automation on a Symphony X peptide synthesizer (Protein Technologies). Unless noted, all procedures were performed in a 45-mL polypropylene reaction vessel fitted with a bottom frit. The reaction vessel connects to the Symphony X peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N2 gas through the bottom frit; the pulse lasts approximately 5 seconds and occurs every 30 seconds. A “single shot” mode of addition describes the addition of all the solution contained in the single shot falcon tube that is usually any volume less than 5 mL. Amino acid solutions were generally not used beyond two weeks from preparation. HATU solution was used within 14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis:

Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony X Method” describe an experiment performed on a 0.050 mmol scale, where the scale is determined by the amount of Sieber or Rink or 2-chlorotrityl or PL-FMP bound to the resin. This scale corresponds to approximately 70 mg of the Sieber amide resin described above. All procedures can be scaled beyond or under 0.050 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- or D-Leu used the “Double-coupling procedure” or the “Single-Coupling 2-Hour Procedure” described below. Unless otherwise specified, the last step of automated synthesis is the acetyl group installation described as “Chloroacetyl Anhydride Installation”. All syntheses end with a final rinse and drying step described as “Standard final rinse and dry procedure”.

### Resin-Swelling Procedure:

To a 45-mL polypropylene solid-phase reaction vessel was added Sieber amide resin (70 mg, 0.050 mmol). The resin was washed (swelled) three times as follows: to the reaction vessel was added DMF (5.0 mL) through the top of the vessel “DMF top wash” upon which the mixture was periodically agitated for 3 minutes before the solvent was drained through the frit.

### Single-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling 4 Equivalent Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling 4 Equivalent Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure A:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, then the vessel was closed. The automatic program was resumed and HATU (0.4 M in DMF, 1.0 mL, 8 equiv) and NMM (0.8 M in DMF, 1.0 mL, 16 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, followed by the manual addition of HATU (2-4 equiv, same equiv as the unnatural amino acid), then the vessel was closed. The automatic program was resumed and NN (0.8 M in DMF, 1.0 mL, 16 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure C.

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) containing HATU (an equimolor amount relative to the unnatural amino acid), and NMM (4-8 equiv) was added manually using a pipette from the top of the vessel while the bottom of the vessel remained attached to the instrument. The automatic program was resumed and the mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure D:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) containing DIC (an equimolor amount relative to the unnatural amino acid), and HOAt (an equimolor amount relative to the unnatural amino acid) was added manually using a pipette from the top of the vessel while the bottom of the vessel remained attached to the instrument. The automatic program was resumed and the mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine: DMF (20:80 v/v, 3.0 mL). The mixture was periodically agitated for 5 minutes and then the solution was drained through the frit. To the reaction vessel was added piperidine: DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added bromoacetic acid (0.4 M in DMF, 1.0 mL, 8 eq), then DIC (0.4 M in DMF, 1.0 mL, 8 eq). The mixture was periodically agitated for 1 hour, then the reaction solution was drained through the frit. The resin was washed successively two times as follows: for each wash, DMF (4.0 mL) was added through the top of the vessel and the resulting mixture was periodically agitated for 30 seconds before the solution was drained through the frit. To the reaction vessel was added the amine (0.4 M in DMF, 2.0 mL, 16 eq). The mixture was periodically agitated for 1 hour, then the reaction solution was drained through the frit. The resin was washed successively five times as follows: for each wash, DMF (3.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 3.0 mL). The mixture was periodically agitated for 3.5 or 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for 1.0 minute before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for 1.0 minute before the solution was drained through the frit. The resulting resin was used directly in the next step.

### Final Rinse and Dry Procedure:

The resin from the previous step was washed successively six times as follows: for each wash, DCM (5.0 mL) was added through the top of the vessel and the resulting mixture was periodically agitated for 30 seconds before the solution was drained through the frit. The resin was then dried using a nitrogen flow for 10 minutes. The resulting resin was used directly in the next step.

### General Deprotection, Cyclization, N-Methylation, Click, Suzuki Procedures:

### Global Deprotection Method A:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL Falcon tube was added the resin and 2.0-5.0 mL of the cleavage cocktail (TFA:TIS:DTT, v/v/w=95:5:1). The volume of the cleavage cocktail used for each individual linear peptide can be variable. Generally, a higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et₂O (30-40 mL); then the mixture was centrifuged for 3-5 minutes; and the solution was decanted away from the solids and discarded. For a final time, the solids were suspended in Et₂O (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 30-ml bio-rad poly-prep chromatography column was added the resin and 2.0-5.0 mL of the cleavage cocktail (TFA:TIS:H₂O:DTT, v/v/w=94:3:3:1). The volume of the cleavage cocktail used for each individual linear peptide can be variable. Generally, a higher number of protecting groups present in the sidechain of the peptide requires a larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA solution. The mixture was centrifuged for 3-5 minutes, then the solution was decanted away from the solids and discarded. The solids were suspended in Et₂O (35 mL); then the mixture was centrifuged for 3-5 minutes; and the solution was decanted away from the solids and discarded. For a final time, the solids were suspended in Et₂O (35 mL); the mixture was centrifuged for 3-5 minutes; and the solution was decanted away from the solids and discarded to afford the crude peptide as a white to off-white solid after drying under a flow of nitrogen and/or under house vacuum. The crude was used the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the global deprotection were dissolved in DMF (30-45 mL) in the 50-mL centrifuge tube at room temperature, and to the solution was added DIEA (1.0-2.0 mL) and the pH value of the reaction mixture above was 8. The solution was then allowed to shake for several hours or overnight or over 2-3 days at room temperature. The reaction solution was concentrated to dryness on a speedvac or genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in a CH₃CN/0.1 M aqueous solution of ammonium bicarbonate (1:1,v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on a speedvac or genevac EZ-2. The resulting residue was charged with CH₃CN:H₂O (2:3, v/v, 30 mL), and concentrated to dryness on a speedvac or genevac EZ-2. This procedure was repeated (usually 2 times). The resulting crude solids were then dissolved in DMF or DMF/DMSO or CH₃CN/H₂O/formic acid. After filtration, the solution was subjected to single compound reverse-phase HPLC purification to afford the desired cyclic peptide.

N-Methylation on-Resin Method A.

To the resin (50 μmol) in a Bio-Rad tube was added CH₂Cl₂ (2 mL) and shaken for 5 min at rt. 2-Nitrobenzene-1-sulfonyl chloride (44.3 mg, 200 μmol, 4 equiv) was added followed by the addition of 2,4,6-trimethylpyridine (0.040 mL, 300 μmol, 6 equiv). The reaction was shaken at rt for 2 h. The solvent was drained and the resin was rinsed with CH₂Cl₂ (5 mL×3), DMF (5 mL×3) and then THF (5 mL×3). To the resin was added THE (1 mL). Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 2-16 h. The reaction was repeated. Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 1-16 h. The solvent was drained, and the resin was washed with THE (5 mL×3) and CHCl₃ (5 mL×3). The resin was air dried and used directly in the next step. The resin was shaken in DMF (2 mL). 2-Mercaptoethanol (39.1 mg, 500 umol) was added followed by DBU (0.038 mL, 250 umol, 5 equiv). The reaction was shaken for 1.5 h. The solvent was drained. The resin was washed with DMF (4×). Air dried and used directly in the next step.

N-Methylation On-Resin Method B (Turner, R. A. Et al, Org. Lett., 15(19):5012-5015 (2013)).

All manipulations were performed manually unless noted. The procedure of “N-methylation on-resin Method A” describes an experiment performed on a 0.100 mmol scale, where the scale is determined by the amount of Sieber or Rink linker bound to the resin that was used to generate the peptide. This scale is not based on a direct determination of the quantity of peptide used in the procedure. The procedure can be scaled beyond 0.10 mmol scale by adjusting the described volumes by the multiple of the scale. The resin was transferred into a 25 mL fritted syringe. To the resin was added piperidine:DMF (20:80 v/v, 5.0 mL). The mixture was shaken for 3 min. and then the solution was drained through the frit. The resin was washed 3 times with DMF (4.0 mL). To the reaction vessel was added piperidine:DMF (20:80 v/v, 4.0 mL). The mixture was shaken for 3 min. and then the solution was drained through the frit. The resin was washed successively three times with DMF (4.0 mL) and three times with DCM (4.0 mL). The resin was suspended in DMF (2.0 mL) and ethyl trifluoroacetate (0.119 ml, 1.00 mmol), 1,8-diazabicyclo[5.4.0]undec-7-ene (0.181 ml, 1.20 mmol). The mixture was placed on a shaker for 60 min. The solution was drained through the frit. The resin was washed successively three times with DMF (4.0 mL) and three times with DCM (4.0 mL). The resin was washed three times with dry THE (2.0 mL) to remove any residual water. In an oven dried 4.0 mL vial was added THE (1.0 mL) and triphenylphosphine (131 mg, 0.500 mmol) and dry 4 Å molecular sieves (20 mg). The solution was transferred to the resin and diisopropyl azodicarboxylate (0.097 mL, 0.5 mmol) was added slowly. The resin was stirred for 15 min. The solution was drained through the frit and the resin was washed three times with dry THE (2.0 mL) to remove any residual water. In an oven dried 4.0 mL vial was added THE (1.0 mL), triphenylphosphine (131 mg, 0.50 mmol) and dry 4 A molecular sieves (20 mg). The solution was transferred to the resin and diisopropyl azodicarboxylate (0.097 mL, 0.5 mmol) was added slowly. The resin was stirred for 15 min. The solution was drained through the frit. The resin was washed successively three times with DMF (4.0 mL) and three times with DCM (4.0 mL). The resin was suspended in Ethanol (1.0 mL) and THE (1.0 mL), and sodium borohydride (37.8 mg, 1.000 mmol) was added. The mixture was stirred for 30 min. and drained. The resin was washed successively three times with DMF (4.0 mL) and three times with DCM (4.0 mL).

### N-Alkylation On-Resin Procedure Method A:

A solution of the alcohol corresponding to the alkylating group (0.046 g, 1.000 mmol), triphenylphosphine (0.131 g, 0.500 mmol), and DIAD (0.097 mL, 0.500 mmol) in 3 mL of THF was added to nosylated resin (0.186 g, 0.100 mmol), and the reaction mixture was stirred for 16 hours at room temperature. The resin was washed three times with THE (5 mL), and the above procedure was repeated 1-3 times. Reaction progress was monitored by TFA micro-cleavage of small resin samples treated with a solution of 50 μL of TIS in 1 mL of TFA for 1.5 hours.

### N-Alkylation On-Resin Procedure Method B:

The nosylated resin (0.100 mmol) was washed three times with N-methylpyrrolidone (NMP) (3 mL). A solution of NMP (3 mL), alkyl bromide (20 eq, 2.000 mmol) and DBU (20 eq, 0.301 mL, 2.000 mmol) was added to the resin, and the reaction mixture was stirred for 16 hours at room temperature. The resin was washed with NMP (3 mL) and the above procedure was repeated once more. Reaction progress was monitored by TFA micro-cleavage of small resin samples treated with a solution of 50 μL of TIS in 1 mL of TFA for 1.5 hours.

### N-Nosylate Formation Procedure:

A solution of collidine (10 eq.) in DCM (2 mL) was added to the resin, followed by a solution of Nos-Cl (8 eq.) in DCM (1 mL). The reaction mixture was stirred for 16 hours at room temperature. The resin was washed three times with DCM (4 mL) and three times with DMF (4 mL). The alternating DCM and DMF washes were repeated three times, followed by one final set of four DCM washes (4 mL).

### N-Nosylate Removal Procedure:

The resin (0.100 mmol) was swelled using three washes with DMF (3 mL) and three washes with NMP (3 mL). A solution of NMP (3 mL), DBU (0.075 mL, 0.500 mmol) and 2-mercaptoethanol (0.071 mL, 1.000 mmol) was added to the resin and the reaction mixture was stirred for 5 minutes at room temperature. After filtering and washing with NMP (3 mL), the resin was re-treated with a solution of NMP (3 mL), DBU (0.075 mL, 0.500 mmol) and 2-mercaptoethanol (0.071 mL, 1.000 mmol) for 5 minutes at room temperature. The resin was washed three times with NMP (3 mL), four times with DMF (4 mL) and four times with DCM (4 mL), and was placed back into a Symphony reaction vessel for completion of sequence assembly on the Symphony peptide synthesizer.

### General Procedure for Preloading Amines on the PL-FMP Resin:

PL-FMP resin (Novabiochem, 1.00 mmol/g substitution) was swollen with DMF (20 mL/mmol) at room temperature. The solvent was drained and 10 ml of DMF was added, followed by the addition of the amine (2.5 mmol) and acetic acid (0.3 mL) into the reaction vessel. After 10-min agitation, sodium triacetoxyhydroborate (2.5 mmol) was added. The reaction was allowed to agitate overnight. The resin was washed with DMF (1×), THF/H₂O/AcOH (6:3:1) (2×), DMF (2×), DCM (3×), and dried. The resulting PL-FMP resin preloaded with the amine can be checked by the following method: Take 100 mg of the above resin and react with benzoyl chloride (5 equiv), and DIEA (10 equiv) in DCM (2 mL) at room temperature for 0.5 h. The resin was washed with DMF (2×), MeOH (1×), and DCM (3×). The sample was then cleaved with 40% TFA/DCM (1 h). The product was collected and analyzed by HPLC and MS. Collected sample was dried and the weight was determine to calculate resin loading.

### General Procedure for Preloading Fmoc-Amino Acids on Cl-Trityl Resin:

To a glass reaction vessel equipped with a frit was added the 2-Chloro-chlorotrityl resin mesh 50-150, (1.54 meq/gram, 1.94 grams, 3.0 mmole) to be swollen in DCM (5 mL) for 5 minutes. A solution of the acid (3.00 mmol, 1.0 eq) in DCM (5 mL) was added to the resin followed by DIEA (2.61 ml, 15.00 mmol, 5.0 eq). The reaction was shaken at room temperature for 60 minutes. DIEA (0.5 mL) and methanol (3 mL) were added, and the reaction was shaken for an additional 15 minutes. The reaction solution was filtered through the frit and the resin was rinsed with DCM (4×5 mL), DMF (4×5 mL), DCM (4×5 mL), diethyl ether (4×5 mL), and dried using a flow of nitrogen. The resin loading can be determined as follows:

A sample of resin (13.1 mg) was treated with 20% piperidine/DMF (v/v, 2.0 mL) for 10 minutes with shaking. 1 mL of this solution was transferred to a 25.0 mL volumetric flask and diluted with methanol to a total volume of 25.0 mL. A blank solution of 20% piperidine/DMF (v/v, 1.0 mL) was diluted up with methanol in a volumetric flask to 25.0 mL. The UV was set to 301 nm. The absorbance was brought to zero with the blank solution followed by the reading of the sample solution, give the absorbance of 1.9411. Loading of the resin was measured to be 0.6736 mmol/g ((1.9411/20 mg)×6.94=0.6736).

### Click Reaction On-Resin Method A:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swollen with DCM (2×5 mL×5 mins) and then DMF (2×5 mL×5 mins). In a 200-ml bottle was charged with a volume of 30 times of the following: ascorbic acid (vitamin C, 0.026 g, 0.150 mmol), bis(2,2,6,6-tetramethyl-3,5-heptanedionato)copper(II) (10.75 mg, 0.025 mmol), DMF (1.5 mL), 2,6-lutidine (0.058 mL, 0.50 mmol) and THE (1.5 mL), followed by DIEA (0.087 ml, 0.50 mmol) and the corresponding azide used in the examples (1.0-2.0 equiv.). The mixture was stirred until everything was in solution. The DMF in the above Bio-Rad tube was drained, and the above click solution (3 mL each) was added to each Bio-Rad tube. The tubes were shaken overnight on an orbital shaker. Solutions were drained through the frit. The resins were washed with DMF (3×2 mL) and DCM (3×2 mL).

### Click Reaction On-Resin Method B:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swollen with DCM (2×5 mL×5 mins) and then DMF (2 5 mL×5 mins). In a separate bottle, nitrogen was bubbled into 4.0 mL of DMSO for 15 mins. To the DMSO was added copper iodide (9.52 mg, 0.050 mmol, 1.0 eq) (sonicated), lutidine (58 μL, 0.500 mmol, 10.0 eq) and DIEA (87 uL, 0.050 mmol, 10.0 eq). The solution was purged with nitrogen again. DCM was drained through the frit. In a separate vial, ascorbic acid (8.8 mg, 0.050 mmol, 1.0 eq) was dissolved into water (600 uL). Nitrogen was bubbled through the solution for 10 mins. Coupling partners were distributed in the tubes (0.050 mmol to 0.10 mmol, 1.0 to 2.0 eq) followed by the DMSO copper and base solution and finally ascorbic acid aqueous solution. The solutions were topped with a blanket of nitrogen and capped. The tubes were put onto the rotatory mixer for 16 hours. Solutions were drained through the frit. The resins were washed with DMF (3×2 mL) and DCM (3×2 mL).

### Suzuki Reaction On-Resin Procedure:

In a Bio Rad tube was placed 50 umoles of dried Rink resin of a N-terminus Fmoc-protected linear polypeptide containing a 4-bromo-phenylalanine side chain. The resin was swelled with DMF (2×5 mL). To this was added a DMF solution (2 mL) of p-tolylboronic acid (0.017 g, 0.125 mmol), potassium phosphate (0.2 mL, 0.400 mmol) followed by the catalyst [1,1′-bis(di-tert-butylphosphino)ferrocene]dichloropalladium(II) [PdCl₂(dtbpf)] (3.26 mg, 5.00 μmol). The tube was shaken at rt overnight. The solution was drained and the resin was washed with DMF (5×3 mL) followed by alternating DCM (2×3 mL), then DMF (2×3 mL), and then DCM (5×3 mL). A small sample of resin was micro-cleaved using 235 μL of TIS in 1 ml TFA at rt for 1 h. The rest of the resin was used in the next step of peptide coupling or chloroacetic acid capping of the N-terminus.

### Solution Phase Click Reaction Method A:

To a 20-ml scintillation vial was added 100-fold of the needed amount of sodium ascorbate (sodium (R)-2-((S)-1,2-dihydroxyethyl)-4-hydroxy-5-oxo-2,5-dihydrofuran-3-olate) and copper(II) sulfate pentahydrate (CuSO₄: sodium ascorbate mol ratio: 1:3 to 1:5). The reaction was diluted with water. This solution was shaken at RT for 1-10 min. The resulting yellowish slurry was added to the respective reactions.

To the vial containing the alkyne and the azide (1.0-2.0 equiv) was added the needed amount of the above copper solution (CuSO₄: 0.3-1.0 equiv of the alkyne). The mixture was shaken at rt for 1-3 h and the progress was monitored by LC/MS. Additional amounts of azide or copper solution can be added to drive the triazole formation if required. After completion, the mixture was diluted with CH₃CN:aq. NH₄CO₃ solution (v/v 1:1), filtered, and purified the same day via reverse phase HPLC purification.

### Solution Phase Click Reaction Method B:

A stock solution of CuSO₄ and sodium ascorbate was prepared by diluting a dry 1:2 to 1:3 mol ratio of copper(II) sulfate pentahydrate and sodium ascorbate to a concentration of 0.1-0.3 M with respect to copper sulfate pentahydrate. To a solution of the peptide alkyne in DMF (0.05-0.1 M) was added the corresponding azide used in the examples (1.0-2.0 equiv) followed by the above freshly prepared aqueous copper solution (0.03-1.0 equiv). The mixture was stirred at room temperature and monitored by LCMS. Additional amounts of azide or copper solution can be added to drive the triazole formation if required. Upon full conversion, the mixture was diluted, filtered and purified the same day by reverse phase HPLC.

### General Purification Procedures:

The crude material was purified via preparative LC/MS using the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at certain percentage of B, then a linear increase from this percentage to a higher percentage of B over 20-30 minutes, then a 0-minute hold at 100% B; Flow Rate: 20-40 mL/min; Column Temperature: 25° C. Fraction collection was triggered by MS and UV signals. Fractions containing the desired product were combined and dried via centrifugal evaporation. If the material was not pure based on the orthogonal analytical data, it was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at certain percentage of B, then a linear increase from the starting percentage of B over 20-30 minutes, then a 0-minute hold at 100% B; Flow Rate: 20-40 mL/min; Column Temperature: 25° C. Fraction collection was triggered by MS signals. Fractions containing the desired product were combined and dried via centrifugal evaporation. The yield and the purity of the product were determined.

Alternatively, based on the initial analytical data, the crude material was purified via preparative LC/MS using the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at certain percentage of B, then a linear increase from the starting percentage of B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. If the material was not pure based on the orthogonal analytical data, it was further purified via preparative LC/MS using the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at certain percentage of B, then a linear increase from this percentage to a higher percentage of B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield and the purity of the product were determined.

### Unnatural Amino Acid Synthesis:

### Preparation of (S)-2-((((9H-fluoren-9-yl)methoxy)carbonyl)-3-(1-(2-(tert-butoxy)-2-oxoethyl)-1H-indol-3-yl)propanoic acid

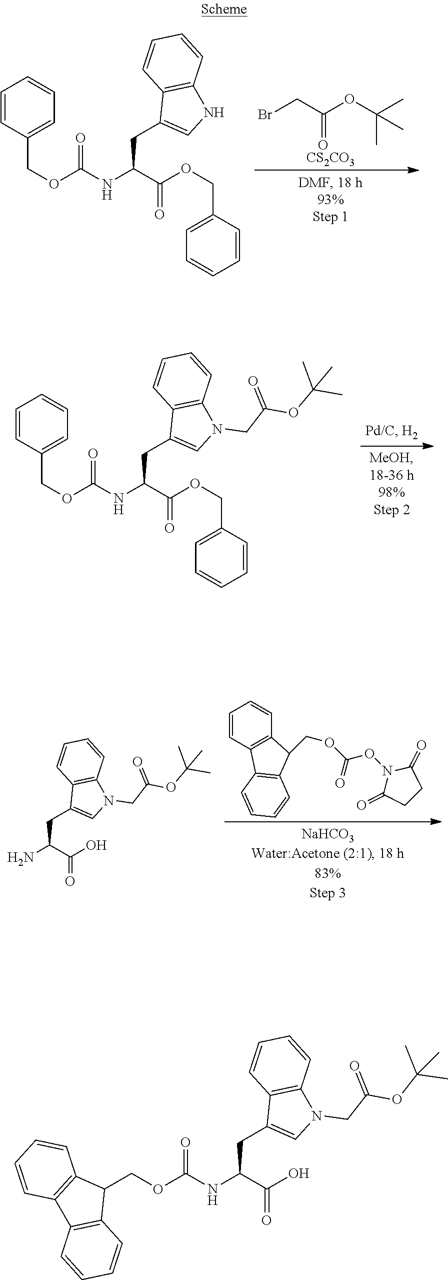

### Step 1:

To a 0° C. solution of (S)-benzyl 2-(((benzyloxy)carbonyl)amino)-3-(1H-indol-3-yl) propanoate (25.0 g, 58.3 mmol) and cesium carbonate (20.9 g, 64.2 mmol) in DMF (200 mL) was added tert-butyl 2-bromoacetate (9.36 mL, 64.2 mmol). The solution was allowed to slowly warm up to RT with stirring for 18 h. The reaction mixture was poured into ice water:aq. 1N HCl (1:1) and then extracted with EtOAc. The organic layer was washed with brine, collected, dried over MgSO₄, filtered, and then concentrated in vacuo. The resulting solid was subjected to flash chromatography (330 g column, 0-50% EtOAc:Hex over 20 column volumes) to afford (S)-benzyl 2-(((benzyloxy)carbonyl)amino)-3-(1-(2-(tert-butoxy)-2-oxoethyl)-1H-indol-3-yl)propanoate as a white solid (29.6 g, 93%).

### Step 2:

H₂ was slowly bubbled through a mixture of (S)-benzyl 2-(((benzyloxy)carbonyl)amino)-3-(1-(2-(tert-butoxy)-2-oxoethyl)-1H-indol-3-yl)propanoate (29.6 g, 54.5 mmol) and Pd—C (1.45 g, 1.36 mmol) in MeOH (200 mL) at RT for 10 min. The mixture was then stirred under positive pressure of H₂ while conversion was monitored by LCMS. After 48 h the reaction mixture was filtered through diatomaceous earth and evaporated to afford crude (S)-2-amino-3-(1-(2-(tert-butoxy)-2-oxoethyl)-1H-indol-3-yl)propanoic acid (17.0 g) which was carried into step three without additional purification.

### Step 3:

To a solution of (S)-2-amino-3-(1-(2-(tert-butoxy)-2-oxoethyl)-1H-indol-3-yl)propanoic acid (5.17 g, 16.2 mmol) and sodium bicarbonate (6.8 g, 81 mmol) in acetone:water (50.0 mL:100 mL) was added (9H-fluoren-9-yl)methyl (2,5-dioxopyrrolidin-1-yl) carbonate (5.48 g, 16.2 mmol). The mixture stirred overnight upon which LCMS analysis indicated complete conversion. The vigorously stirred mixture was acidified via slow addition of aq 1N HCl. Once acidified, the mixture was diluted with DCM (150 mL), and the isolated organic phase was then washed with water, followed by brine. The organic layer was collected, dried over sodium sulfate, and concentrated under vacuum to afford the crude product. The crude material was purified via silica gel chromatography (330 g column, 20-80% EtOAc:Hex over 20 column 25 volumes) to afford (S)-2-((((9H-fluoren-9-yl)methoxy)carbonyl)amino)-3-(1-(2-(tertbutoxy)-2-oxoethyl)-1H-indol-3-yl)propanoic acid as a white foam (7.26 g, 83%). ¹H NMR (500 MHz, methanol-d4) δ 7.80 (d, J=7.6 Hz, 2H), 7.67-7.60 (m, 2H), 7.39 (t, J=7.5 Hz, 2H), 7.32-7.22 (m, 3H), 7.18 (td, J=7.6, 0.9 Hz, 1H), 7.08 (td, J=7.5, 0.9 Hz, 1H), 7.04 (s, 1H), 4.54 (dd, J=8.4, 4.9 Hz, 1H), 4.36-4.23 (m, 2H), 4.23-4.14 (m, 1H), 30 3.43-3.35 (m, 2H), 3.25-3.09 (m, 1H), 1.55-1.38 (m, 9H). ESI-MS(+) m/z=541.3 (M+H).

### Preparation of (S)-2-((((9H-fluoren-9-yl)methoxy)carbonyl)amino)-3-(4-(2-(tert-butoxy)-2-oxoethoxy)phenyl)propanoic acid

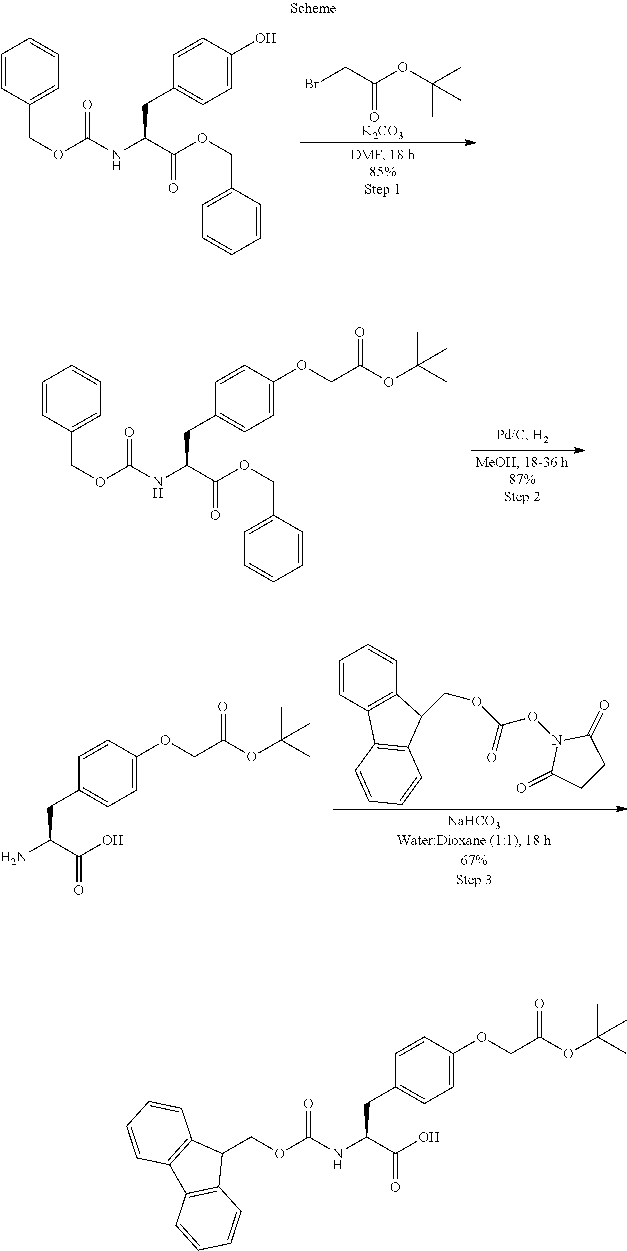

### Step 1:

To a cooled stirred solution of (S)-benzyl 2-(((benzyloxy)carbonyl)amino)-3-(4-hydroxyphenyl)propanoate (70 g, 173 mmol) and K₂CO₃ (35.8 g, 259 mmol) in DMF (350 mL) was added tert-butyl-2-bromoacetate (30.6 mL, 207 mmol) dropwise and the resulting mixture was stirred at RT overnight. The reaction mixture was diluted with 10% brine solution (1000 mL) and extracted with ethyl acetate (2×250 mL). The combined organic layer was washed with water (500 mL), saturated brine solution (500 mL), dried over anhydrous sodium sulfate, filtered, and concentrated to afford a colorless gum. The crude compound was purified by flash column chromatography using 20% ethyl acetate in petroleum ether as an eluent to afford a white solid (78 g, 85%).

### Step 2:

The (S)-benzyl 2-(((benzyloxy)carbonyl)amino)-3-(4-(2-(tert-butoxy)-2-oxoethoxy)phenyl)propanoate (73 g, 140 mmol) was dissolved in MeOH (3000 mL) and purged with nitrogen for 5 min. To the above purged mixture was added Pd/C (18 g, 16.91 mmol) and stirred under hydrogen pressure of 3 kg for 15 hours. The reaction mixture was filtered through a bed of diatomaceous earth (Celite©) and washed with methanol (1000 mL). The filtrate was concentrated under vacuum to afford a white solid (36 g, 87%).

### Step 3:

To a stirred solution of (S)-2-amino-3-(4-(2-(tert-butoxy)-2-oxoethoxy)phenyl)propanoic acid (38 g, 129 mmol) and sodium bicarbonate (43.2 g, 515 mmol) in water (440 mL) was added Fmoc-OSu (43.4 g, 129 mmol) dissolved in dioxane (440 mL) dropwise and the resulting mixture was stirred at RT overnight. The reaction mixture was diluted with 1.5 N HCl (200 mL) and water (500 mL) and extracted with ethyl acetate (2×250 mL). The combined organic layer was washed with water (250 mL), saturated brine solution (250 mL), and dried over Na₂SO₄, filtered, and concentrated to afford a pale yellow gum. The crude compound was purified by column chromatography using 6% MeOH in chloroform as an eluent to afford a pale green gum. The gum was further triturated with petroleum ether to afford an off-white solid (45 g, 67%). ¹H NMR (400 MHz, DMSO-d6) δ 12.86-12.58 (m, 1H), 7.88 (d, J=7.5 Hz, 2H), 7.73-7.61 (m, 3H), 7.58-7.47 (m, 1H), 7.44-7.27 (m, 4H), 7.18 (d, J=8.5 Hz, 2H), 6.79 (d, J=8.5 Hz, 2H), 4.57 (s, 2H), 4.25-4.10 (m, 4H), 3.34 (br s, 3H), 3.02 (dd, J=13.8, 4.3 Hz, 1H), 2.81 (dd, J=14.1, 10.5 Hz, 1H), 1.41 (s, 9H).

### Linker/Tail Synthesis:

### Preparation of (2S)-4-[(35-azido-3,6,9,12,15,18,21,24,27,30,33-undecaoxapentatriacontan-1-yl)carbamoyl]-2-(16-sulfohexadecanamido)butanoic acid (IUPAC) (4, Ns-PEG11-γGlu-FSA16)

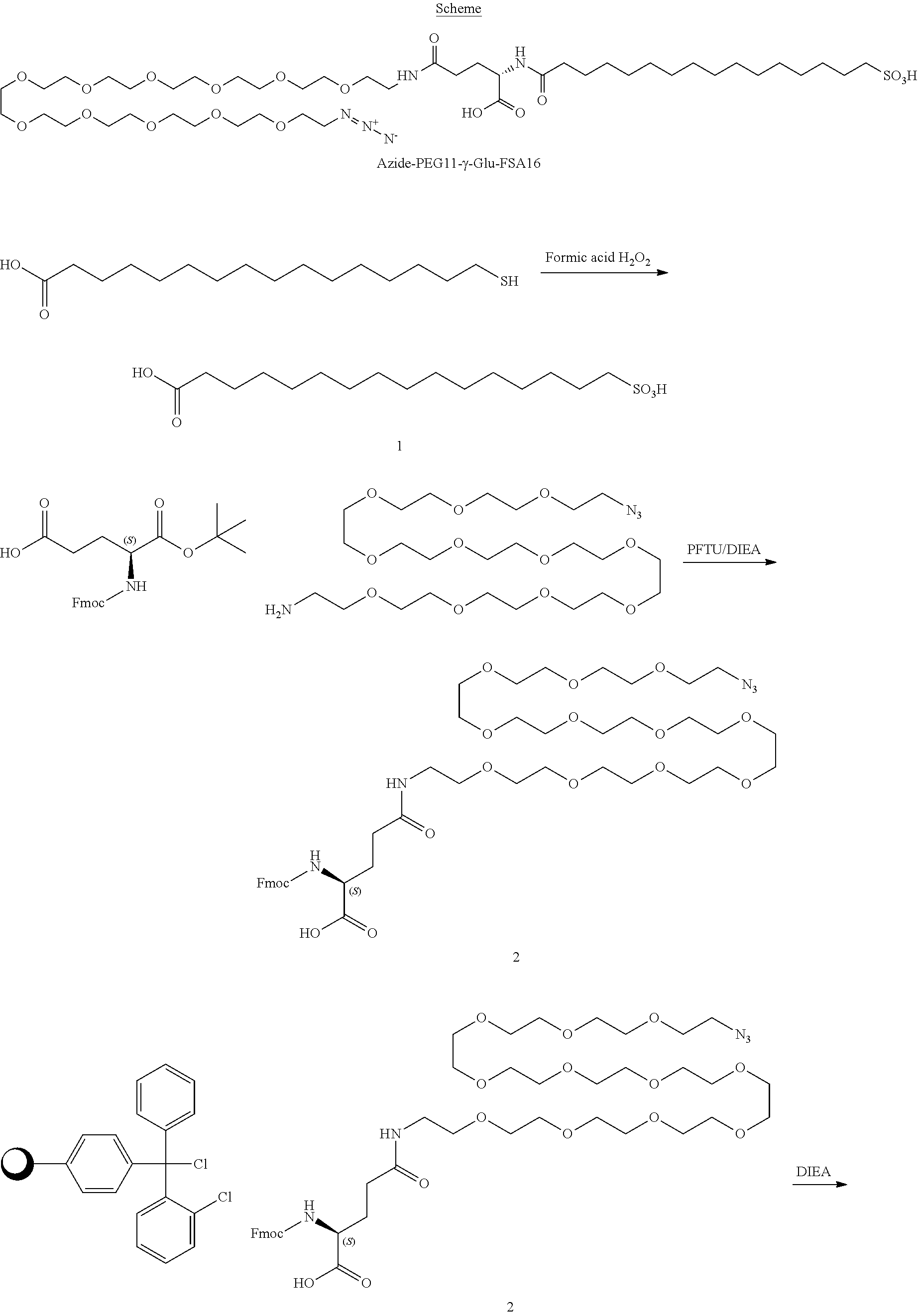

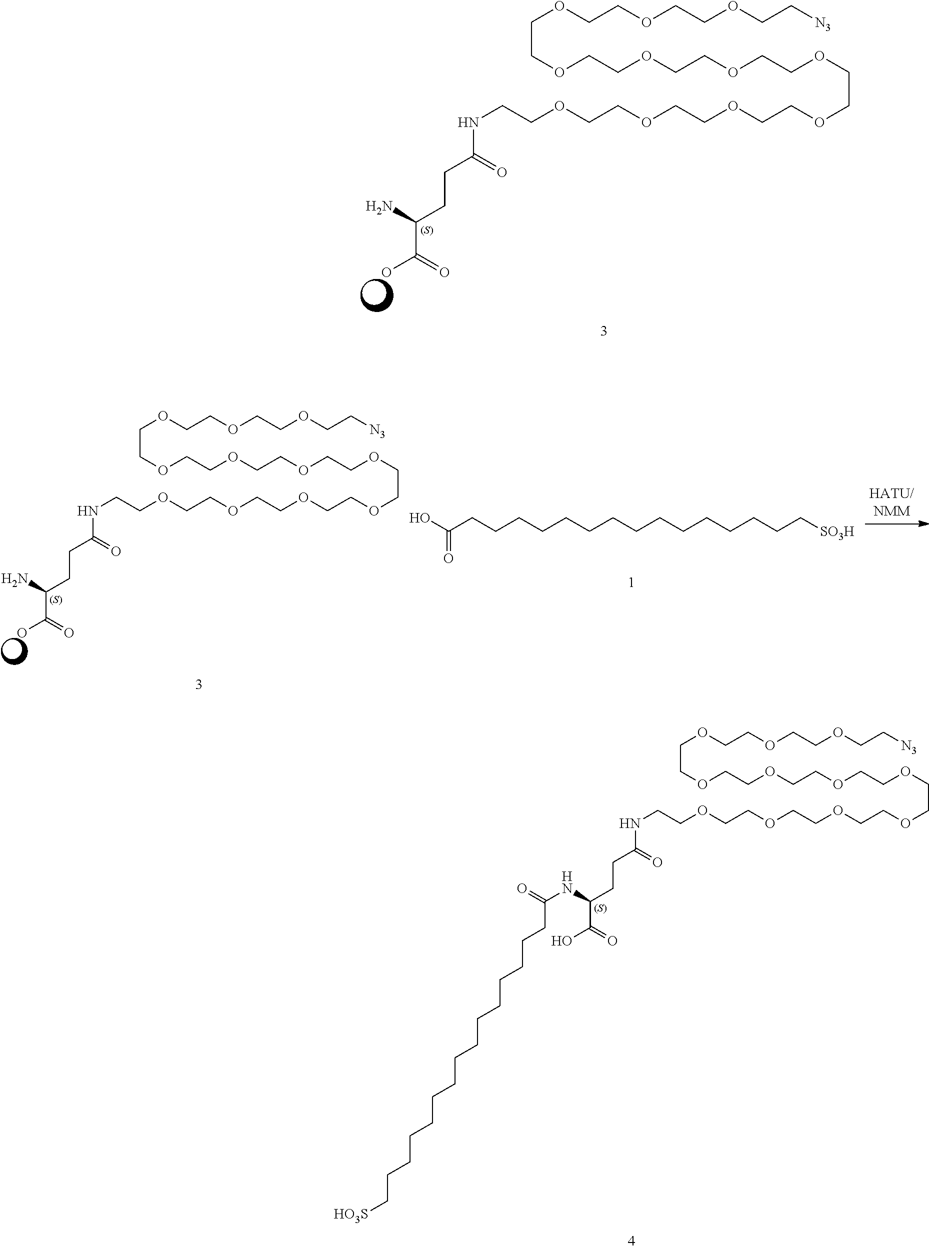

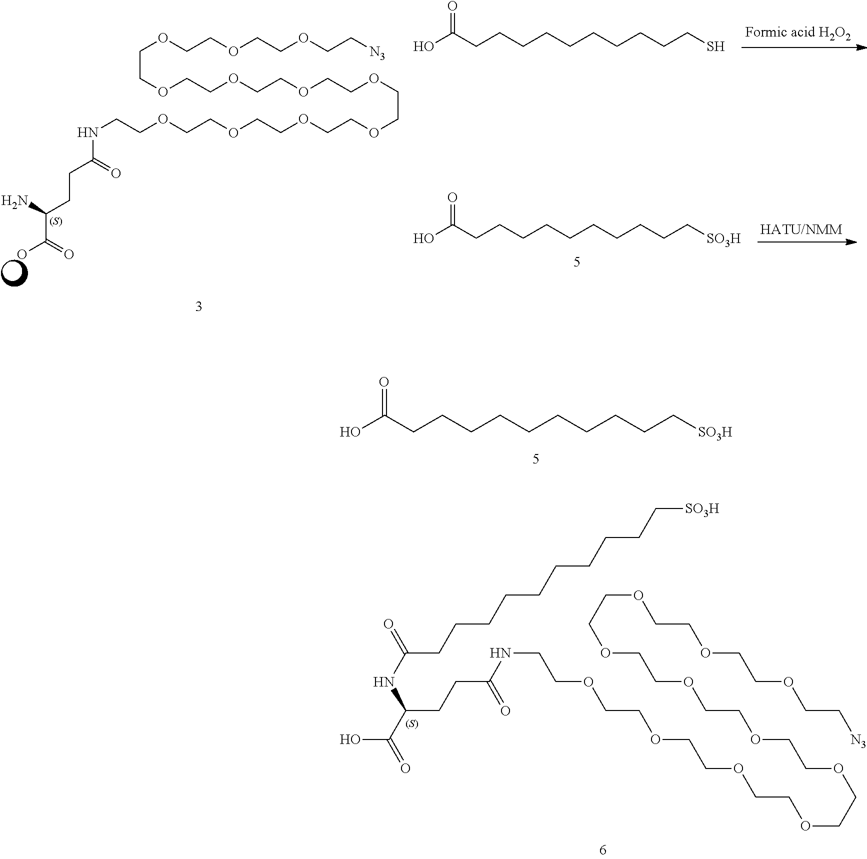

### Step 1:

16-Mercaptohexadecanoic acid (5.3 g, 18.37 mmol) was added to a 500-mi round-bottomed flask with a stir bar. Formic acid (172 ml, 4485 mmol) was added followed by dropwise addition of hydrogen peroxide (12.3 ml, 120 mmol). The mixed suspension was stirred for 6 hours open to the air. Ran a gentle stream of nitrogen over the sample overnight. After 16 hours, the product was a nice white powder. Pumped on for 6 hours and then used as is. Isolated 6.18 g of compound 1. Analysis LCMS condition O: Retention time=1.30 min; ESI-MS(+) m/z 337.0 (M+1)

Elaborated thiol oxidation step with reductive workup: To a stirring slurry of 16-mercaptohexadecanoic acid (5.07 g, 17.57 mmol) in formic acid (176 ml) was added hydrogen peroxide (11.76 ml, 115 mmol) dropwise via addition funnel (over ˜ 5-10 min). It was stirred at rt for 6 h. Peroxide test strips test positive above reaction surface, intense blue. Upon submerging in reaction solution, they turn yellow (not described on color scale). LCMS shows desired product present and no detectable amount of starting material (programmed MW values, no UV signals, AA the +/−ion mode). Cool reaction solution with an ice bath and equip with 2-way nitrogen inlet adapter attached to manifold. Fit with a stopper, begin adding dimethyl sulfide (3.90 ml, 52.7 mmol) through stopper dropwise via syringe (˜1 mL/min). Test with strips, positive above surface and yellow below surface. Begin slow addition of remaining dimethyl sulfide (0.715 ml, 9.67 mmol) and monitor with test strips. As full addition is neared, test strips show no indication above reaction solution and test positive upon submerge. With ˜0.1 mL sulfide remaining, strips no longer indicate/change color. Remove ice bath, stir 1 h. Concentrate under strong stream of nitrogen over weekend. Concentrate in vacuo on high vacuum for 48 h, isolate: 7.63 g. NMR consistent with product structure shows residual solvents (DMSO and water mainly, trace dimethylsulfide). Use with a nominal w % of 75% to account for residual solvents.

### Step 2:

A dry mixture of Fmoc-Glu-OtBu (1.640 g, 3.86 mmol) and PFTU (1.651 g, 3.86 mmol) was diluted with DMF (15 ml). While stirring under nitrogen, DIEA (1.347 ml, 7.71 mmol) was slowly added via syringe. After stirring for 1 h, 35-azido-3,6,9,12,15,18,21,24,27,30,33-undecaoxapentatriacontan-1-amine (2.0 g, 3.50 mmol) was added using minimal DMF to quantitate transfer. After stirring for 1.5 h at RT, reaction was quenched with 10% LiCl and extracted once with EtOAc. Resulting concentrate was purified by normal phase ISCO eluting with 90/10 DCM/MeOH. Resulting oil was diluted with 10 ml of DCM and then treated with 15 ml of TFA. After ¾ hours, reaction was concentrated to dryness and purified by ISCO chromatography eluting with 85/15 DCM/MeOH. Isolated 3.20 g of product as compound 2. Analysis LCMS condition O: Retention time=1.40 min; ESI-MS(+) m/z 923.08 (M+1)

### Step 3:

Chloro Trityl Resin (4334 mg, 13.88 mmol) was washed with 5×DCM and then diluted with DCM (20 mL). To this solution was then added 2 (3200 mg, 3.47 mmol) followed by DIEA (4.85 mL, 27.8 mmol). Reaction immediately turned grape color and was allowed to shake for 1.5 hours. The resin was then diluted with 20 ml of a 9:1 Methanol/Hunigs base solution and quickly filtered and washed with 3×DCM and 3×DMF. The resulting brownish purple resin was then treated with 2×20% piperidine/DMF to deprotect the Fmoc group, washed 5×DMF and used as is. Resin turned yellow in color. Cleaved aliquot of resin with 20% HFIPA/DCM. LCMS indicates desired product on resin 3. Analysis LCMS condition O: Retention time=0.83 min; ESI-MS(+) m/z 700.08 (M+1)

### Step 4:

Resin 3 (5.74 g, 2.87 mmol) was washed with 5×DMF and then diluted with DMF (40 mL). To this solution was then added a solution of 1 (1.642 g, 4.88 mmol) and HATU (1.855 g, 4.88 mmol) in DMF (40 mL) followed by N-methylmorpholine (2.52 mL, 22.96 mmol). Reaction was allowed to shake for 16 hours and then washed with 5×DMF and 5×DCM. Cleaved product off resin with a 30-min treatment of 20% HFIPA/DCM. Drained and concentrated to give 2.92 g of 4.

Analysis LCMS condition O: Retention time=1.30 min; ESI-MS(+) m/z 1019.08 (M+1). ¹H NMR (400 MHz, DMSO) δ 1.25 (s, 22H), 1.50 (m, 2H), 1.80 (m, 2H), 1.90 (m, 2H), 2.10 (m, 5H), 2.38 (m, 2H), 3.20 (m, 2H), 3.40 (m, 2H), 3.50 (m, 40H), 3.60 (m, 2H), 4.20 (m, 1H), 5.20 (m, 1H), 8.10 (s, 1H), 12.50 (m, 1H). Yield (4 steps): 96%.

### Preparation of (S)-1-azido-37-oxo-40-(11-sulfoundecanamido)-3,6,9,12,15,18,21,24,27,30,33-undecaoxa-36-azahentetracontan-41-oic acid (6)

### Step 1:

11-Mercaptoundecanoic acid (5.0 g, 22.90 mmol) was added to a 500-ml round-bottomed flask with a stir bar. Formic acid (214 mL, 5587 mmol) was added followed by dropwise addition of hydrogen peroxide (15.32 mL, 150 mmol). The mixed suspension was stirred for 6 h open to the air. A gentle stream of nitrogen was bubbled over the reaction mixture. After 16 h, the product was a nice white powder. It was pumped on for 6 h and then used as is. Isolated 6.10 g of compound 5. Analysis LCMS condition P: Retention time=1.03 min; ESI-MS(+) m/z 267.0 (M+1)

### Step 2:

Resin 3 (4.00 g, 2.00 mmol) was washed with 5×DMF and then diluted with DMF (40 mL). To this solution was then added a solution of 5 (0.906 g, 3.40 mmol) and HATU (1.293 g, 3.40 mmol) in DMF (40 mL) followed by N-methylmorpholine (1.759 mL, 16.00 mmol). The reaction was allowed to shake for 16 h and then washed with 5×DMF and 5×DCM. The product was cleaved off the resin with a 30-min treatment of 20% HFIPA/DCM. The solution was drained and concentrated to give 3.12 g of 6. Analysis LCMS condition P: Retention time=1.29 min; ESI-MS(+) m/z 949.0 (M+1). Yield (4 steps): 96%.

### Preparation of 14-mercaptotetradecanoic acid

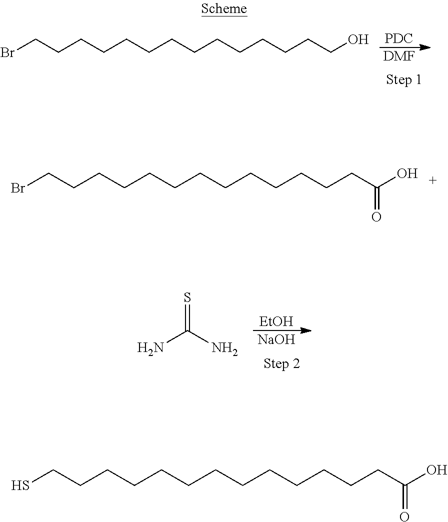

### Step 1.

PDC (10.26 g, 27.3 mmol) dissolved in DMF (34.1 ml) was added to 14-bromotetradecan-1-ol (2.0 g, 6.82 mmol) in DMF (34.1 ml). The resulting mixture was stirred at room temperature overnight and most of the starting alcohol is consumed. Water was added and the resulting mixture is extracted with CH2Cl2. The organic phase was washed with brine, dried over MgSO4 and concentrated in vacuum to give crude 14-bromotetradecanoic acid which was taken to the next step as is. 1H NMR (499 MHz, CHLOROFORM-d) δ 3.55-3.27 (m, 2H), 2.49-2.17 (m, 2H), 1.97-1.77 (m, 2H), 1.71-1.56 (m, 2H), 1.49-1.11 (m, 18H).

### Step 2.

The mixture of 14-bromotetradecanoic acid (2.096 g, 6.82 mmol)) and thiourea (0.880 g, 11.56 mmol) in Ethanol (42.6 ml) was refluxed under nitrogen atmosphere for 1 day. Cooled off the mixture to room temperature and evaporate the solvent under reduced pressure. Heated the residue with NaH (20 mL, 7.5 mol/L) for another 6 h. Acidified the mixture carefully with HCl (1 mol/L). Separated the organic layer. Extracted the aqueous phase with CH2Cl2. The combined organic layers were washed with brine, dried over MgSO4 and concentrated in vacuum to provide 14-mercaptotetradecanoic acid which was taken to the next step as is. 1H NR (499 MHz, CHILOROFORM-d) δ 2.76-2.66 (m, 1H), 2.58-2.50 (m, 1H), 2.41-2.34 (m, 2H), 1.72-1.60 (c, 4H), 1.42-1.23 (i, 18H).

The following compounds were prepared according to same procedures described previously.

| Structure | Name | LCMS |
|---|---|---|
| 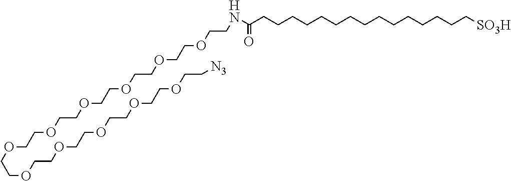 [SMILES: [N-]=[N+]=NCCOCCOCCOCCOCCOCCOCCOCCOCCOCCOCCOCCNC(=O)CCCCCCCCCCCCCCCS(=O)(=O)O] | 1-azido-37-oxo- 3,6,9,12,15,18,21,24,27,30,33- undecaoxa-36-azadopentacontane- 52-sulfonic acid (ELN) 15-[(35-azido- 3,6,9,12,15,18,21,24,27,30,33- undecaoxapentatriacontan-1- yl)carbamoyl]pentadecane-1- sulfonic acid (IUPAC) (N₃-PEG11-FSA16) | Analysis condition A: Retention time = 1.68 min; ESI- MS(+) m/z [M − H]⁻: 887.0 |
| 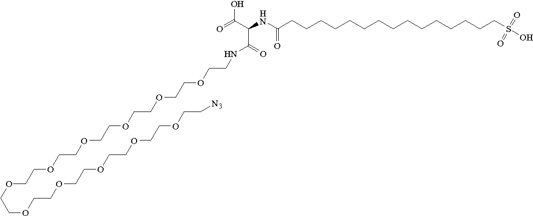 [SMILES: [N-]=[N+]=NCCOCCOCCOCCOCCOCCOCCOCCOCCOCCOCCOCCNC(=O)[C@H](NC(=O)CCCCCCCCCCCCCCCS(=O)(=O)O)C(=O)O] | N-(35-azido- 3,6,9,12,15,18,21,24,27,30,33- undecaoxapentatriacontyl) stearamide (ELN) (2S)-4-[(35-azido- 3,6,9,12,15,18,21,24,27,30,33- undecaoxapentatriacontan-1- yl)carbamoyl]-2-(16- sulfohexadecanamido)butanoic acid (IUPAC) (N₃-PEG11-γGlu-FSA16) | Analysis condition A: Retention time = 1.47 min; ESI- MS(+) m/z [M + H]⁺: 1018.0 |
| 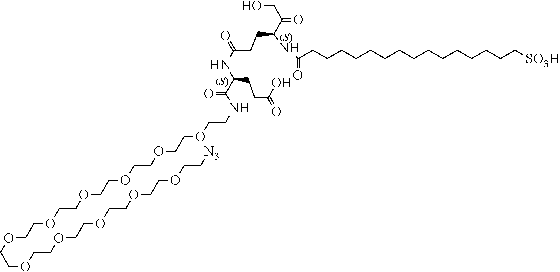 | (38S,43S)-1-azido-38-(2- carboxyethyl)-37,40-dioxo-43- (16-sulfohexadecanamido)- 3,6,9,12,15,18,21,24,27,30,33- undecaoxa-36,39- diazatetratetracontan-44-oic acid (ELN) (2S)-4-[(35-azido-(2S)-4-{[(1S)- 1-[(35-azido- 3,6,9,12,15,18,21,24,27,30,33- undecaoxapentatriacontan-1- yl)carbamoyl]-3- carboxypropyl]carbamoyl}-2-(16- sulfohexadecanamido)butanoic acid (IUPAC) (N₃(Dab)-Glu-γGlu-FDA16) | Analysis condition B: Retention time = 1.35 min; ESI- MS(+) m/z [M + H]⁺: 1147.1 |
| 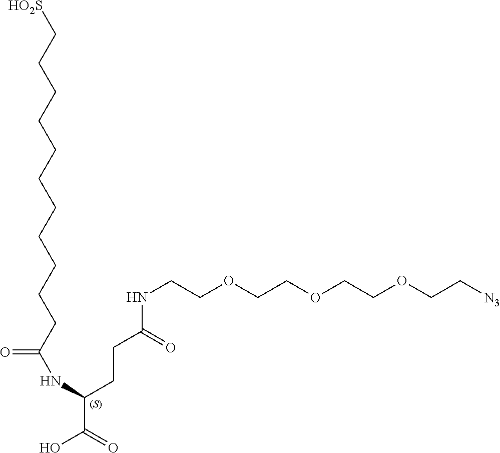 | (S)-1-azido-13-oxo-16-(12- sulfododecanamido)-3,6,9-trioxa- 12-azaheptadecan-17-oic acid (ELN) (2S)-4-[(2-{2-[2-(2- azidoethoxy)ethoxy]ethoxy}ethyl) carbamoyl]-2-(12- sulfododecanamido)butanoic acid (IUPAC) (N₃-PEG3-γGlu-FSA12) | Analysis condition B: Retention time = 1.24 min; ESI- MS(+) m/z [M + H]⁺: 610.3 |
| 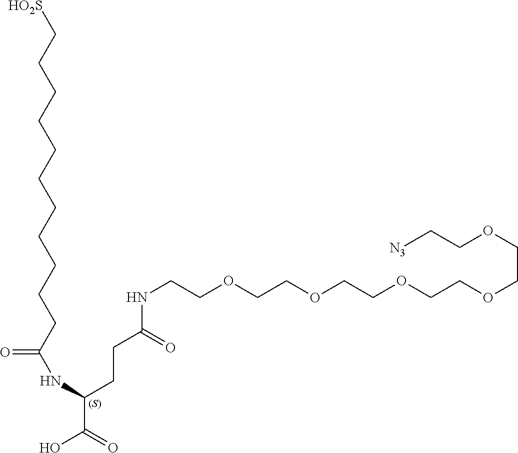 | (S)-1-azido-19-oxo-22-(12- sulfododecanamido)-3,6,9,12,15- pentaoxa-18-azatricosan-23-oic acid (ELN) (2S)-4-[(17-azido-3,6,9,12,15- pentaoxaheptadecan-1- yl)carbamoyl]-2-(12- sulfododecanamido)butanoic acid (IUPAC) (N₃-PEG5-γGlu-FSA12) | Analysis condition B: Retention time = 1.28 min; ESI- MS(+) m/z [M + H]⁺: 698.0 |
| 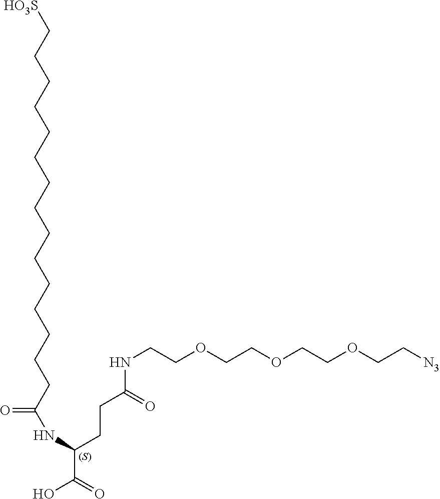 | (S)-1-azido-13-oxo-16-(16- sulfohexadecanamido)-3,6,9- trioxa-12-azaheptadecan-17- oic acid (ELN) (2S)-4-[(2-{2-[2-(2- azidoethoxy)ethoxy]ethoxy}ethyl) carbamoyl]-2-(16- sulfohexadecanamido)butanoic acid (IUPAC) (N₃-PEG3-γGlu-FSA16) | Analysis condition A: ESI- MS(+) m/z [M + H]⁺: 666 |
| 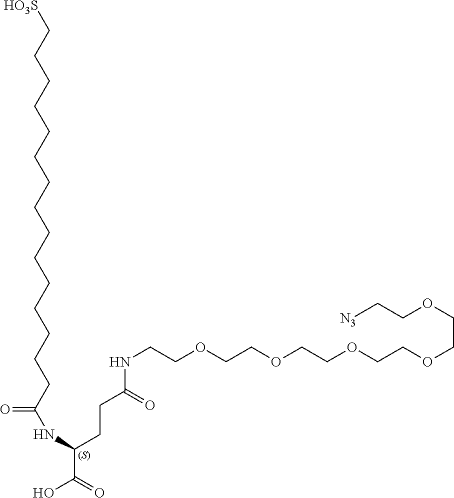 | (S)-1-azido-19-oxo-22-(16- sulfohexadecanamido)- 3,6,9,12,15- pentaoxa-18-azatricosan-23-oic acid (ELN) (9Z)-N-(35-azido-(2S)-4-[(17- azido-3,6,9,12,15- pentaoxaheptadecan-1- yl)carbamoyl]-2-(16- sulfohexadecanamido)butanoic acid (IUPAC) (N₃-PEG5-γGlu-FSA16) | Analysis condition N: ESI- MS(+) m/z [M + H]⁺: 754 |
| 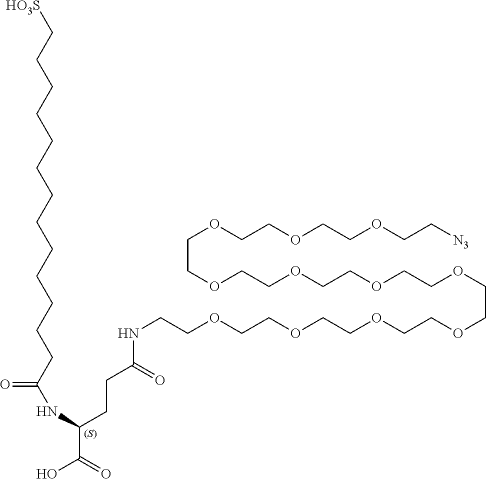 | (S)-1-azido-37-oxo-40-(14- sulfotetradecanamido)- 3,6,9,12,15,18,21,24,27,30,33- undecaoxa-36-azahentetracontan- 41-oic acid (ELN) (2S)-4-[(35-azido- 3,6,9,12,15,18,21,24,27,30,33- undecaoxapentatriacontan-1- yl)carbamoyl]-2-(14- sulfotetradecanamido)butanoic acid (IUPAC) (N₃-PEG5-γGlu-FSA14) | Analysis condition N: ESI- MS(+) m/z [M + H]⁺: 990.3 |
| 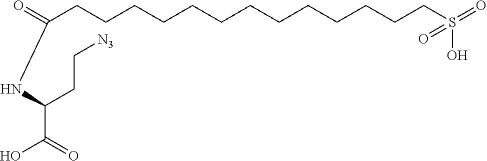 [SMILES: [N-]=[N+]=NCC[C@H](NC(=O)CCCCCCCCCCCCCS(=O)(=O)O)C(=O)O] | (2S)-4-azido-2-(14- sulfotetradecanamido)butanoic Acid (IUPAC) (N₃(Dab)-FSA14) | Analysis condition O: ESI- MS(+) m/z [M + H]⁺: 435.1 |
| 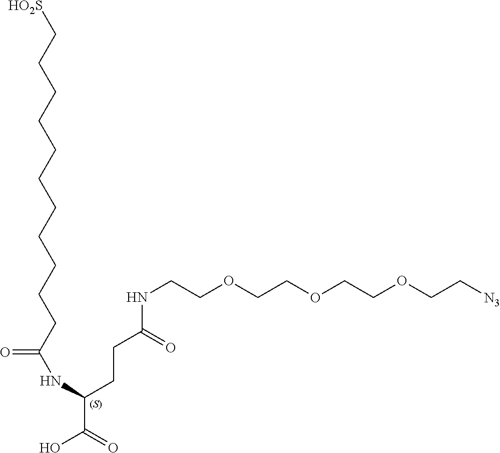 | (2S)-4-[(2-{2-[2-(2- azidoethoxy)ethoxy]ethoxy} ethyl)carbamoyl]-2-(12- sulfododecanamido)butanoic acid (IUPAC) (N₃-PEG3-γGlu-FSA12) | Analysis condition O: ESI- MS(+) m/z [M + H]⁺: 610.6 |

### Synthesis of FPA16 Tail

### Preparation of (S)-1-azido-37-oxo-40-(16-phosphonohexadecanamido)-3,6,9,12,15,18,21,24,27,30,33-undecaoxa-36-azahentetracontan-41-oic acid (3, N₃-PEG11-γGlu-FPA16, A2323-456,457,459)

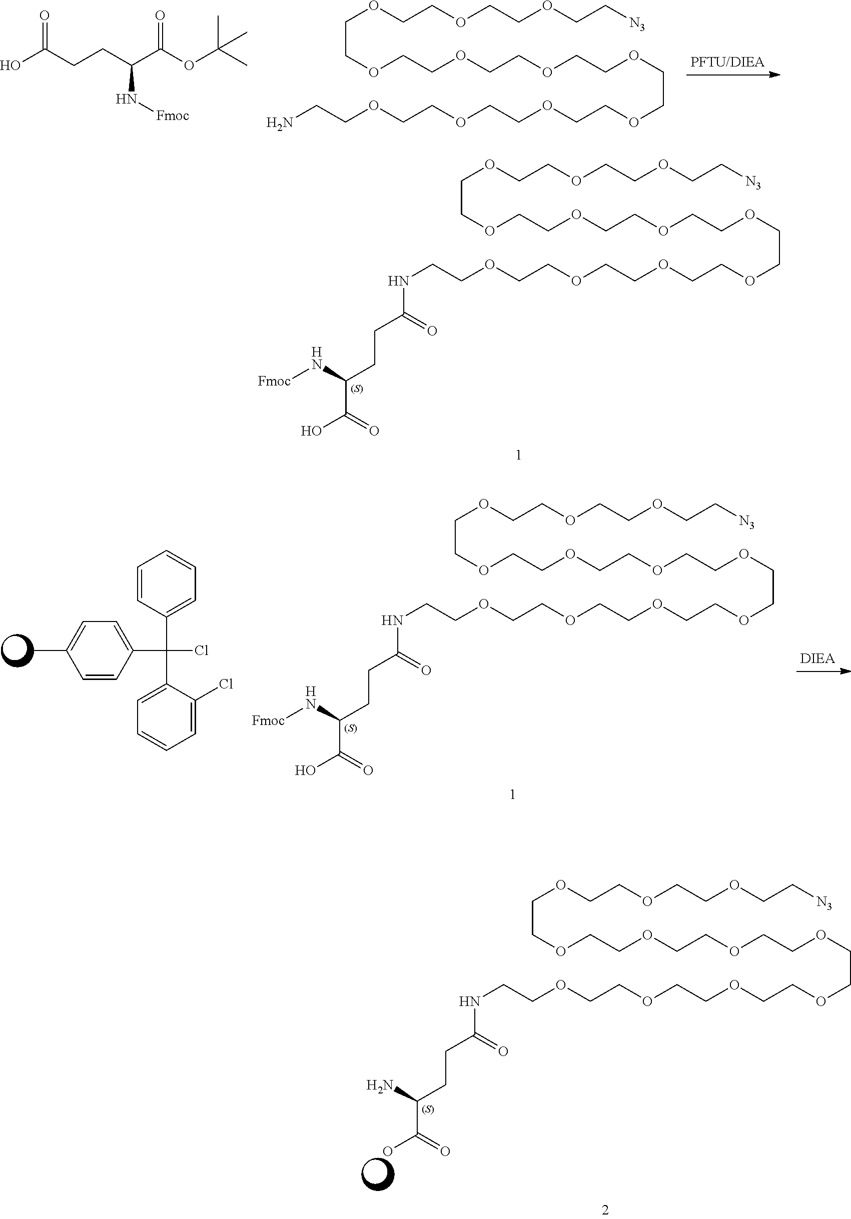

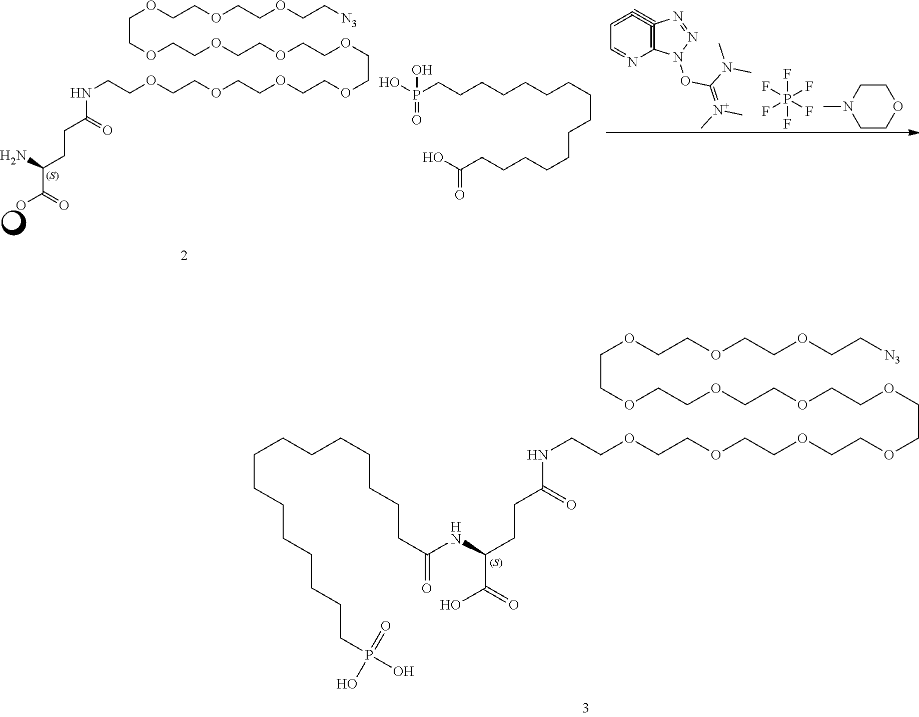

### Step 1:

A dry mixture of Fmoc-Glu-OtBu (2.0 g, 4.70 mmol) and PFTU (2.214 g, 5.17 mmol) was diluted with DMF (15 ml). While stirring under nitrogen, DIEA (1.806 ml, 10.34 mmol) was slowly added via syringe. After stirring for 1 h at rt. 35-azido-3,6,9,12,15,18,21,24,27,30,33-undecaoxapentatriacontan-1-amine (2.68 g, 4.70 mmol) was added using minimal DMF to quantitate transfer.

After stirring for 16 h at rt, the reaction was quenched with 10% LiCl and extracted once with EtOAc. The resulting concentrate was purified by normal phase ISCO eluting with 90/10 DCM/MeOH. The resulting oil was diluted with 10 mL of DCM and then treated with 15 mL of TFA. After ¾ h, the reaction was concentrated to dryness and purified by ISCO chromatography eluting with 90/10 DCM/MeOH to give 3.9 g of product of compound 1. Analysis condition P: Retention time=1.79 min; ESI-MS(+) m/z 922.3 (M+1)

### Step 2:

Chloro Trityl Resin (5282 mg, 16.92 mmol) was washed with 5×DCM and then diluted with DCM (20 mL). To this solution was then added 1 (3900 mg, 4.23 mmol) followed by DIEA (5.91 mL, 33.8 mmol). The reaction immediately turned grape color and was allowed to shake for 1.5 h. The resin was then diluted with 20 mL of a 9:1 Methanol/Hunigs base solution and quickly filtered and washed with 3×DCM and 3×DMF. The resulting brownish purple resin was then treated with 2×20% piperidine/DMF to deprotect the Fmoc group, washed 5×DMF and used as is. The resin turned yellow in color. Cleaved aliquot of resin with 20% HFIPA/DCM. LCMS indicates desired product on resin 2. Analysis condition P: Retention time=1.16 min; ESI-MS(+) m/z 700.3 (M+1)

### Step 3:

Resin 2 (1.0 g, 0.5 mmol) was washed with 5×DMF and then diluted with DMF (40 mL). To this solution was then added a solution of 16-phosphonohexadecanoic acid (0.336 g, 1.000 mmol) and HATU (0.380 g, 1.000 mmol) in DMF (40 mL) followed by N-methylmorpholine (0.440 mL, 4.00 mmol). The reaction was allowed to shake for 16 h and then washed w/5×DMF and 5×DCM. The product was cleaved off of the resin with a 30 minute treatment of 20% HFIPA/DCM. It was drained and concentrated to give 0.50 g of 3. Analysis condition P: Retention time=1.73 min; ESI-MS(+) m/z 1018.4 (M+1). Yield (3 steps): 84%

### Preparation of 15-sulfopentadecanoic acid

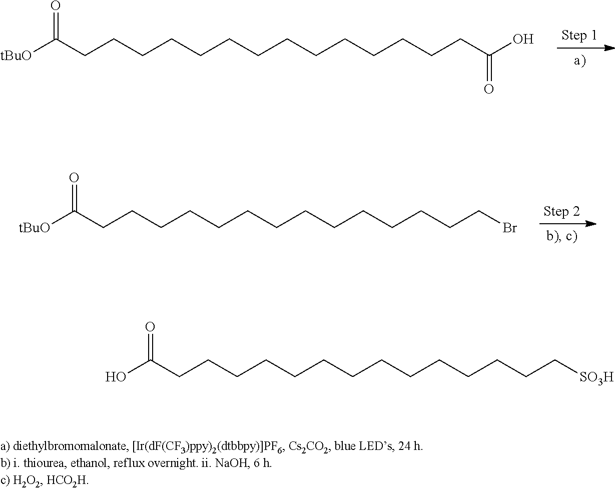 [SMILES: CC(C)(C)OC(=O)CCCCCCCCCCCCCCC(=O)O ; CC(C)(C)OC(=O)CCCCCCCCCCCCCCBr ; O=C(O)CCCCCCCCCCCCCCS(=O)(=O)O]

### Step 1

Decarboxylative halogenation: To a 40 mL pressure relief vial was added 16-(tert-butoxy)-16-oxohexadecanoic acid (512 mg, 1.495 mmol), dimethyl 2-bromomalonate (0.555 mL, 3.74 mmol), [Ir(dF(CF₃)ppy)₂(dtbbpy)]PF₆ (33.5 mg, 0.030 mmol), and cesium carbonate (487 mg, 1.495 mmol). A stir bar was added and the mixture was diluted with chlorobenzene (29.9 mL), capped, and nitrogen was bubbled for 10 min. The vessel was sealed with parafilm and irradiated with blue LED strip lights (lambda max 450, ˜1-2 cm from lights, fan on) with stirring overnight. The vial was centrifuged and the translucent solution was decanted. The reaction mixture was concentrated in vacuo and purified by flash chromatography (0-10% EtOAc/Heptane over 15-20 min). The fractions were monitored by TLC staining with KMnO₄ stain, and the product containing fractions were collected and concentrated to give 324.4 mg of tert-butyl 15-bromopentadecanoate. ¹H NMR (499 MHz, CHLOROFORM-d) δ 3.41 (t, J=6.9 Hz, 2H), 2.21 (t, J=7.6 Hz, 2H), 1.86 (dt, J=14.6, 7.1 Hz, 2H), 1.59 (br d, J=7.4 Hz, 2H), 1.45 (s, 9H), 1.44-1.39 (m, 2H), 1.36-1.20 (m, 18H).

### Step 2:

Thiol formation and oxidation: The above tert-butyl 15-bromopentadecanoate material (292 mg, 0.774 mmol) was treated according to the thiolation procedure for the preparation of 14-mercaptotetradecanoic acid (step 1) to provide 219 mg of product (as a mixture of tBu ester and free carboxylic acid). The crude mixture was then oxidized according to the procedure for the preparation of 16-sulfopentadecanoic acid to provide 241 mg of 15-sulfopentadecanoic acid product. ¹H NMR (499 MHz, METHANOL-d4) δ 4.93 (s, 5H), 2.88-2.79 (m, 2H), 2.35-2.24 (m, 2H), 1.83-1.74 (m, 2H), 1.64-1.56 (m, 2H), 1.47-1.39 (m, 2H), 1.37-1.27 (m, 18H).

### Alkyne Intermediate Synthesis

The following is the detailed example of Pra intermediates used in the solution phase synthesis.

### Preparation of INT-1001

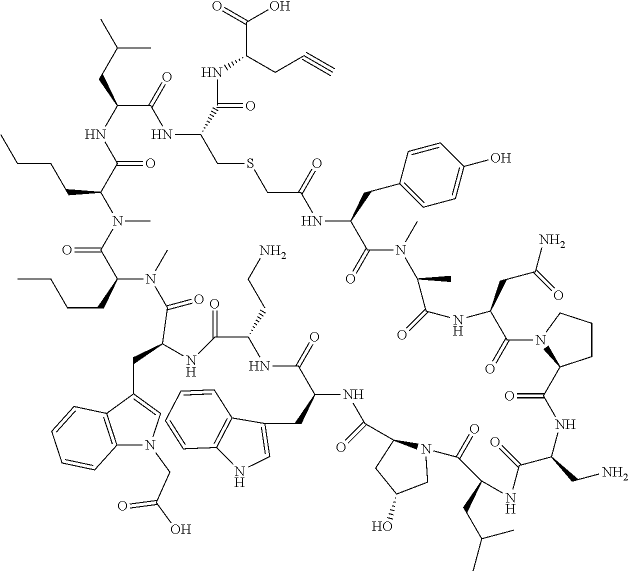 [SMILES: C#CC[C@H](NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1001 was prepared following the general synthetic sequence described for the preparation of Example 0001, composed of the following general procedures. To a 45-mL polypropylene solid-phase reaction vessel was added 2-chlorotrityl resin pre-loaded with Fmoc-Pra-OH on a 100 μmol scale, and the reaction vessel was placed on the Symphony X peptide synthesizer. The following procedures were then performed sequentially:
“Symphony X Resin-Swelling procedure” was followed;
“Symphony X Single-Coupling procedure” was followed with Fmoc-Cys(Trt)-OH;
“Symphony X Single-Coupling procedure” was followed with Fmoc-Leu-OH;
“Symphony X Single-Coupling procedure” was followed with Fmoc-N-Me-Nle-OH;
“Symphony Double-Coupling procedure” was followed with Fmoc-N-Me-Nle-OH;
“Symphony Double-Coupling procedure” was followed with Fmoc-Trp(1-CH₂COOtBu)-OH;
“Symphony X Single-Coupling procedure” was followed with Fmoc-Dab(Boc)-OH;
“Symphony X Single-Coupling procedure” was followed with Fmoc-Trp(Boc)-OH;
“Symphony X Single-Coupling procedure” was followed with Fmoc-Hyp(OtBu)-OH;
“Symphony X Single-Coupling procedure” was followed with Fmoc-Leu-OH;
“Symphony X Single-Coupling procedure” was followed with Fmoc-Dap(Boc)-OH;
“Symphony X Single-Coupling procedure” was followed with Fmoc-Pro-OH;
“Symphony Double-Coupling procedure” was followed with Fmoc-Asn(Trt)-OH;
“Symphony X Single-Coupling procedure” was followed with Fmoc-N-Me-Ala-OH;
“Symphony Double-Coupling procedure” was followed with Fmoc-Tyr(OtBu)-OH;
“Symphony X Chloroacetic Anhydride coupling procedure” was followed;
Alternatively, “Symphony X 4 Equivalent Single-Coupling procedure” or “Symphony X 4 Equivalent Double-Coupling procedure” was used for each step of the peptide elongation;

Alternatively, the above linear sequence was assembled on Prelude peptide synthesizer composed of the following general procedures: “Prelude Resin-Swelling procedure”, “Prelude Single-Coupling procedure” or “Prelude Double-Coupling procedure” (wherein, 4-5 equiv of the amino acid and 90-120 min coupling time was used for each coupling reaction), and “Prelude Chloroacetic Anhydride Coupling procedure”.
“Global Deprotection Method A” was followed;
“Cyclization Method A” was followed.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 25.4 mg, and its estimated purity by LCMS analysis was 100%.
Analysis condition A: Retention time=1.58 min; ESI-MS(+) m/z [M+H]¹⁺: 1926.25.
Analysis condition B: Retention time=1.69 min; ESI-MS(+) m/z [M+2H]²⁺: 963.98.

According to similar procedures as described in INT-1001 and general procedures described above, the following alkyne compounds were synthesized.

### Preparation of INT-1002

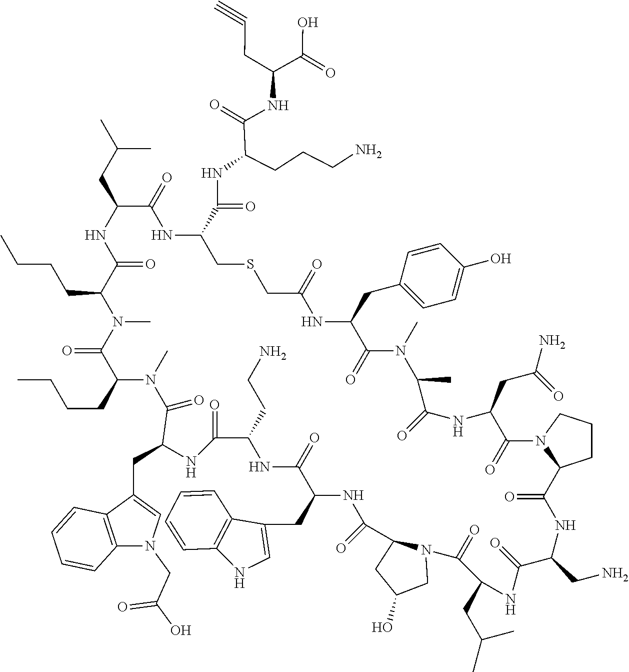 [SMILES: C#CC[C@H](NC(=O)[C@H](CCCN)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1002 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 14% B, 14-54% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 4-minute hold at 100% B; Flow Rate: 40 mL/min; Column Temperature: 25° C. Fraction collection was triggered by MS signals. Fractions containing the desired product were combined and dried via centrifugal evaporation. The yield of the product was 3.1 mg, and its estimated purity by LCMS analysis was 100%.
Analysis condition A: Retention time=1.55 min; ESI-MS(+) m/z [M+2H]²⁺: 1021.2.

### Preparation of INT-1003

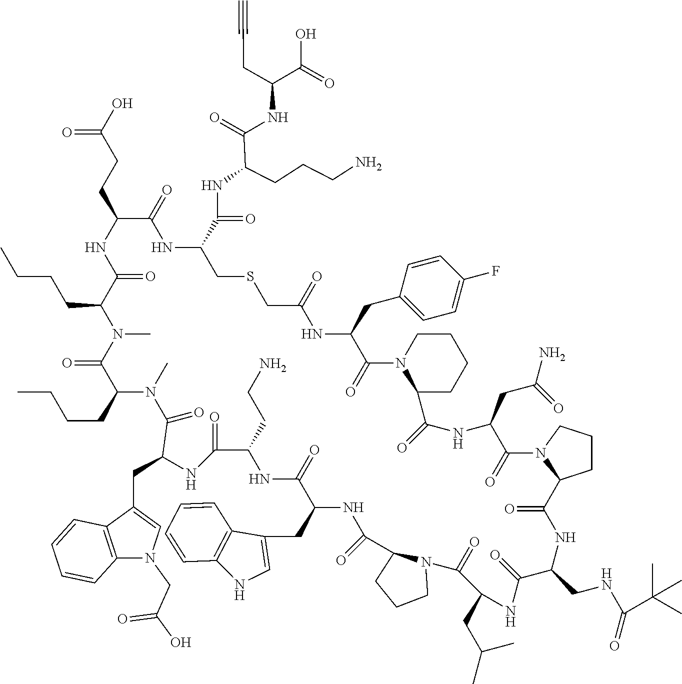 [SMILES: C#CC[C@H](NC(=O)[C@H](CCCN)NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1003 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 25% B, 25-65% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 30% B, 30-70% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 11.6 mg, and its estimated purity by LCMS analysis was 97%.
Analysis condition B: Retention time=1.99 min; ESI-MS(+) m/z [M+2H]²⁺: 1069.4.

### Preparation of INT-1004

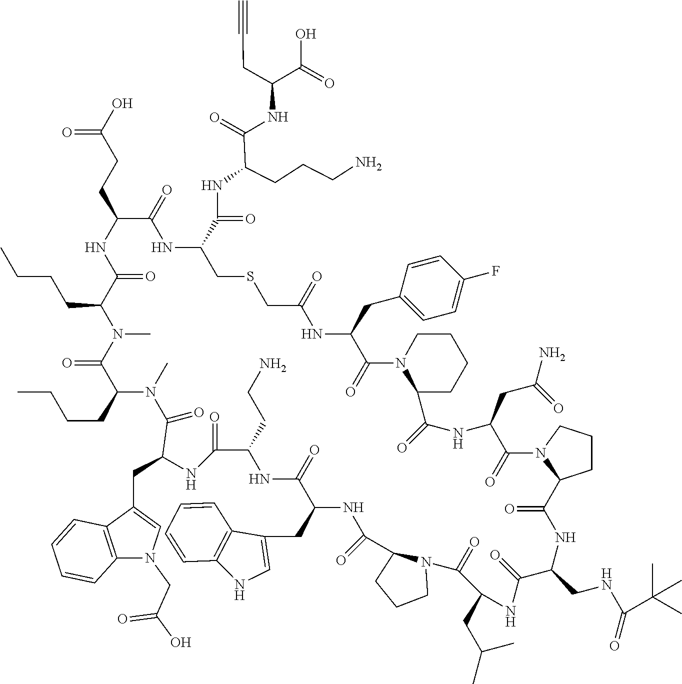 [SMILES: C#CC[C@H](NC(=O)[C@H](CCCN)NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1004 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 26% B, 26-66% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 11.2 mg, and its estimated purity by LCMS analysis was 92.8%.
Analysis condition B: Retention time=1.81, 2.02 min; ESI-MS(+) m/z [M+2H]²⁺: 1077.

### Preparation of INT-1005

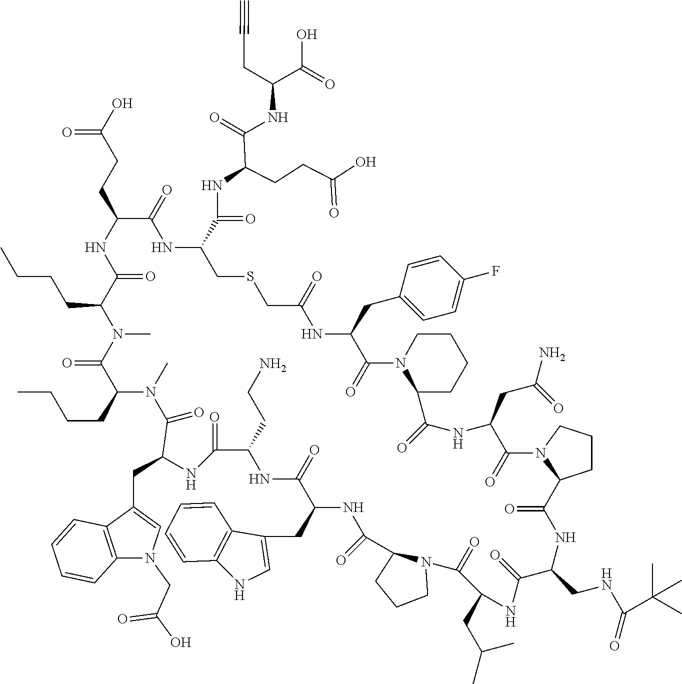 [SMILES: C#CC[C@H](NC(=O)[C@@H](CCC(=O)O)NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1005 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 25% B, 25-65% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 22.4 mg, and its estimated purity by LCMS analysis was 100%.
Analysis condition A: Retention time=1.87 min; ESI-MS(+) m/z [M+2H]²⁺: 1084.4.

### Preparation of INT-1006

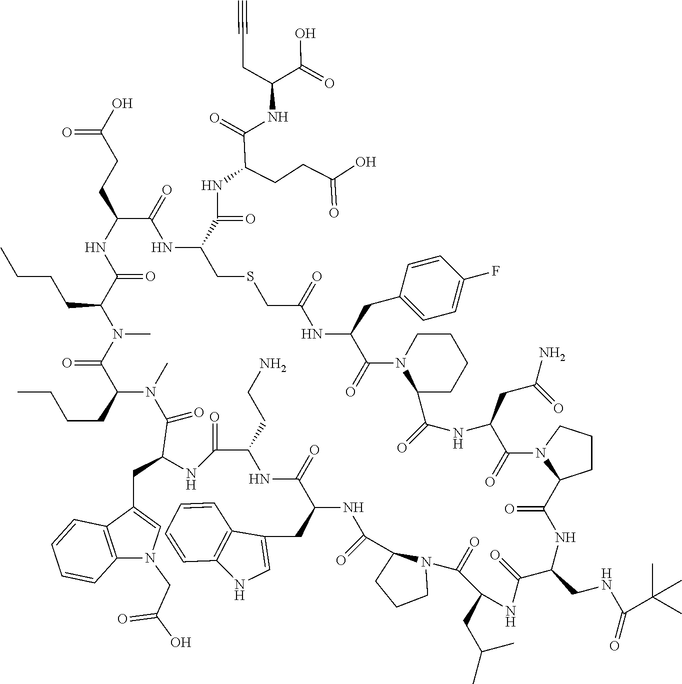 [SMILES: C#CC[C@H](NC(=O)[C@H](CCC(=O)O)NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1006 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 25% B, 25-65% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 18.9 mg, and its estimated purity by LCMS analysis was 100%. Analysis condition A: Retention time=1.71 min; ESI-MS(+) m/z [M+2H]²⁺: 1084.

### Preparation of INT-1007

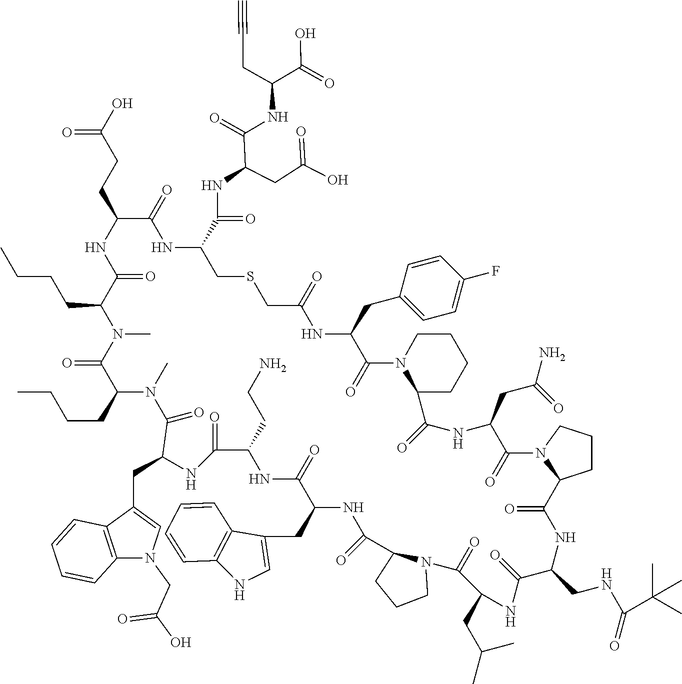 [SMILES: C#CC[C@H](NC(=O)[C@@H](CC(=O)O)NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1007 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 23% B, 23-63% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 26.8 mg, and its estimated purity by LCMS analysis was 99%.
Analysis condition B: Retention time=2.23 min; ESI-MS(+) m/z [M+2H]²⁺: 1077.2.

### Preparation of INT-1008

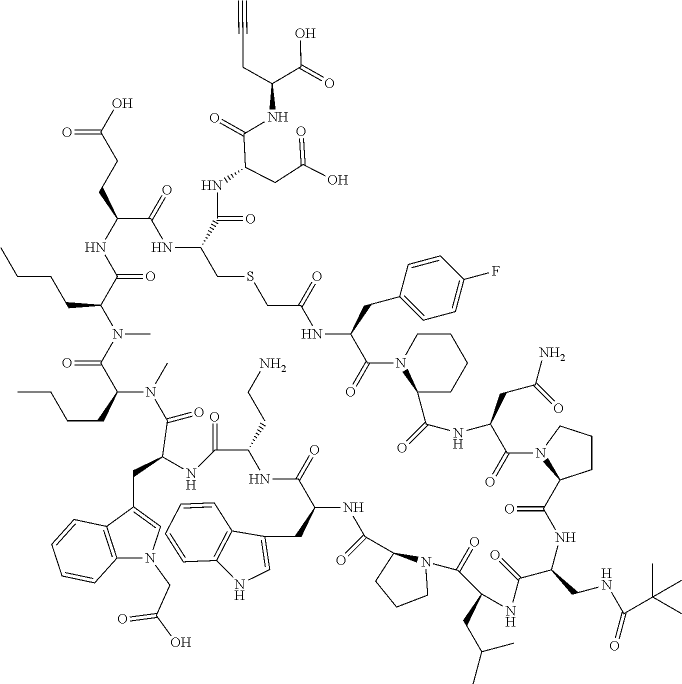 [SMILES: C#CC[C@H](NC(=O)[C@H](CC(=O)O)NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1008 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 23% B, 23-63% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 24.9 mg, and its estimated purity by LCMS analysis was 96.9%.
Analysis condition B: Retention time=2.25 min; ESI-MS(+) m/z [M+2H]²⁺: 1077.4.

### Preparation of INT-1009

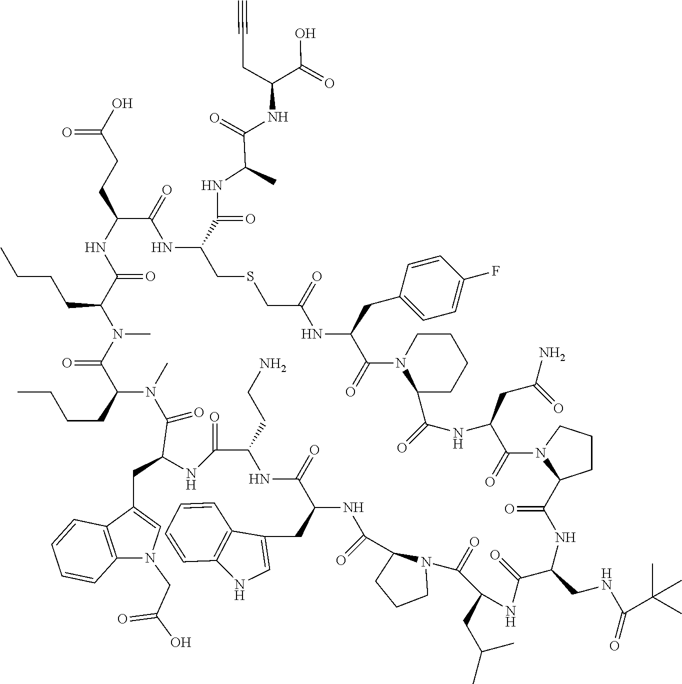 [SMILES: C#CC[C@H](NC(=O)[C@@H](C)NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1009 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 38% B, 38-78% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 36.5 mg, and its estimated purity by LCMS analysis was 100%.
Analysis condition B: Retention time=2.21 min; ESI-MS(+) m/z [M+2H]²⁺: 1055.4.

### Preparation of INT-1010

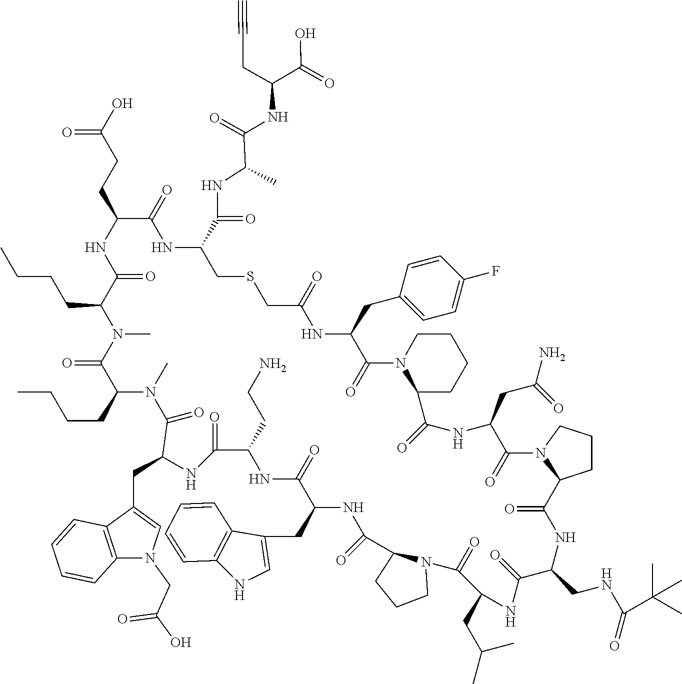 [SMILES: C#CC[C@H](NC(=O)[C@H](C)NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1010 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 26% B, 26-66% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 26.3 mg, and its estimated purity by LCMS analysis was 100%.
Analysis condition A: Retention time=1.92 min; ESI-MS(+) m/z [M+2H]²⁺: 1055.3.

### Preparation of INT-1011

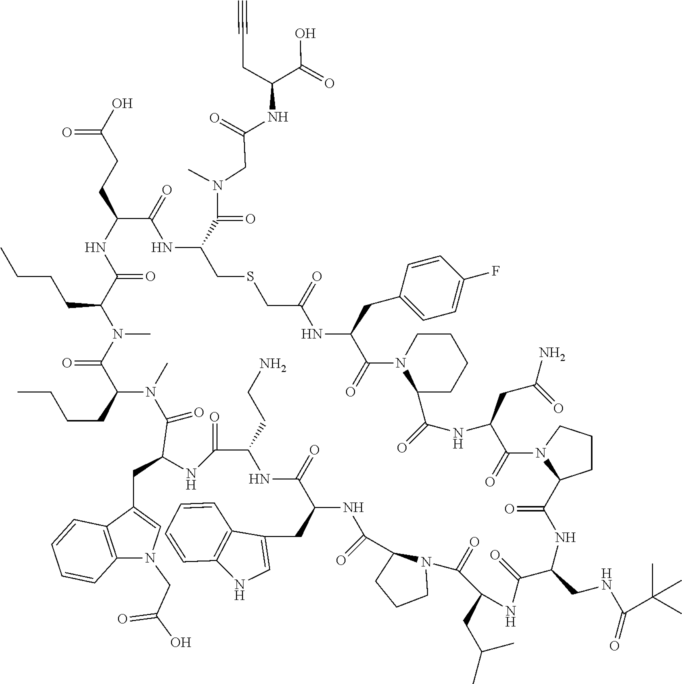 [SMILES: C#CC[C@H](NC(=O)CN(C)C(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1011 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 24% B, 24-64% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 17.7 mg, and its estimated purity by LCMS analysis was 96.9%.
Analysis condition B: Retention time=2.13 min; ESI-MS(+) m/z [M+2H]²⁺: 1055.8.

### Preparation of INT-1012

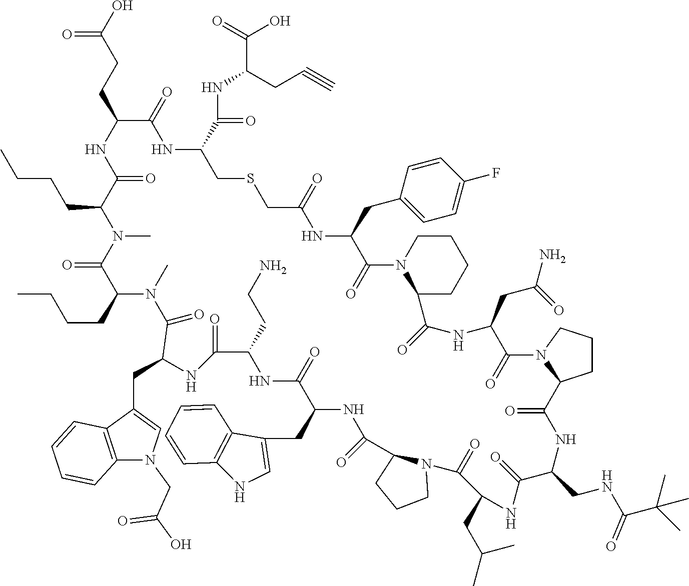 [SMILES: C#CC[C@H](NC(=O)[C@@H]1CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CCC(=O)O)C(=O)N1)C(=O)O]

INT-1012 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 38% B, 38-78% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 16.1 mg, and its estimated purity by LCMS analysis was 98.9%.
Analysis condition B: Retention time=2.18 min; ESI-MS(+) m/z [M+2H]²⁺: 1020.1.

### Preparation of INT-1013

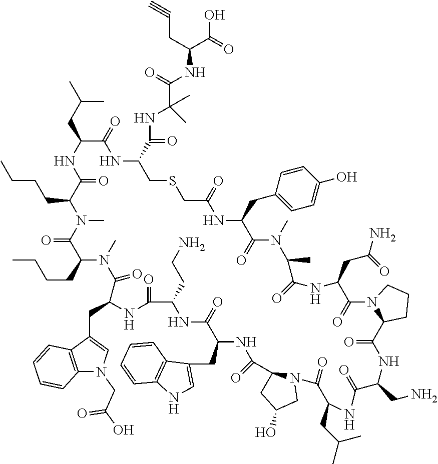 [SMILES: C#CC[C@H](NC(=O)C(C)(C)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1013 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 20.2 mg, and its estimated purity by LCMS analysis was 96.3%.
Analysis condition A: Retention time=1.53 min; ESI-MS(+) m/z [M+2H]²⁺: 1006.2.

### Preparation of INT-1014

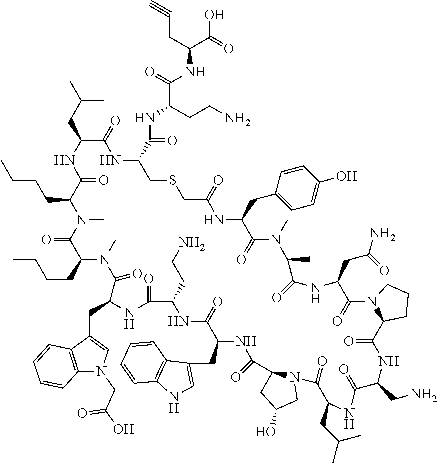 [SMILES: C#CC[C@H](NC(=O)[C@H](CCN)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1014 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 14% B, 14-54% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation.

The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 10.7 mg, and its estimated purity by LCMS analysis was 100%.
Analysis condition A: Retention time=1.5 min; ESI-MS(+) m/z [M+2H]²⁺: 1014.

### Preparation of INT-1015

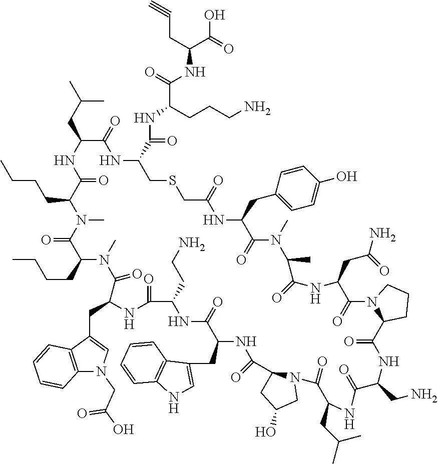 [SMILES: C#CC[C@H](NC(=O)[C@H](CCCN)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1015 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 14% B, 14-54% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 35 mL/min; Column Temperature: 25° C. Fraction collection was triggered by MS signals. Fractions containing the desired product were combined and dried via centrifugal evaporation. The yield of the product was 9.1 mg, and its estimated purity by LCMS analysis was 100%.
Analysis condition A: Retention time=1.55 min; ESI-MS(+) m/z [M+2H]²⁺: 1021.1.

### Preparation of INT-1016

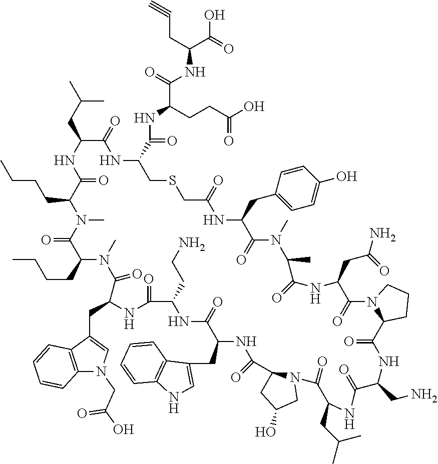 [SMILES: C#CC[C@H](NC(=O)[C@@H](CCC(=O)O)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1016 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 38.6 mg, and its estimated purity by LCMS analysis was 93%.
Analysis condition A: Retention time=1.53 min; ESI-MS(+) m/z [M+2H]²⁺: 1028.4.

### Preparation of INT-1017

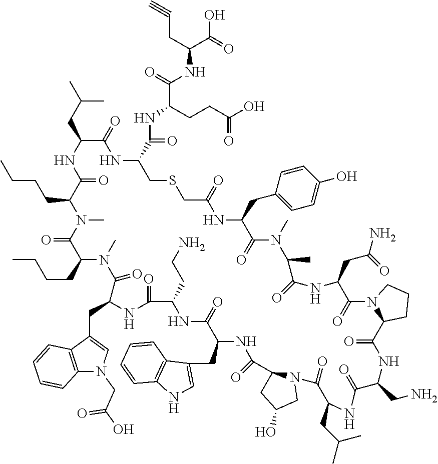 [SMILES: C#CC[C@H](NC(=O)[C@H](CCC(=O)O)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1017 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 38.3 mg, and its estimated purity by LCMS analysis was 90%.
Analysis condition A: Retention time=1.52 min; ESI-MS(+) m/z [M+2H]²⁺: 1028.

### Preparation of INT-1018

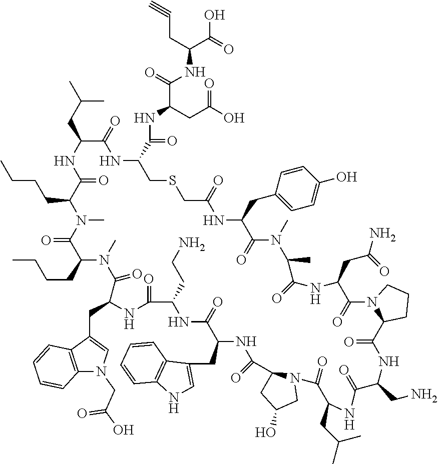 [SMILES: C#CC[C@H](NC(=O)[C@@H](CC(=O)O)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1018 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 22.7 mg, and its estimated purity by LCMS analysis was 92.6%.
Analysis condition A: Retention time=1.43 min; ESI-MS(+) m/z [M+2H]²⁺: 1021.4.

### Preparation of INT-1019

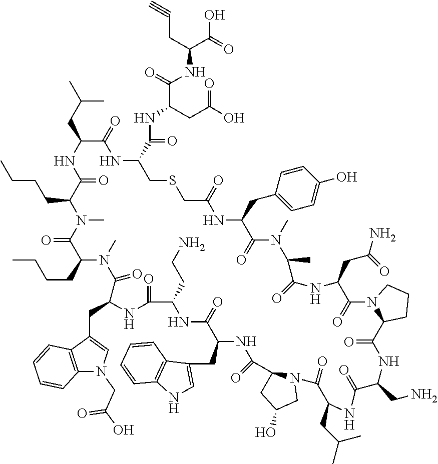 [SMILES: C#CC[C@H](NC(=O)[C@H](CC(=O)O)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1019 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 23.6 mg, and its estimated purity by LCMS analysis was 91.2%.
Analysis condition B: Retention time=1.59 min; ESI-MS(+) m/z [M+3H]³⁺: 681.

### Preparation of INT-1020

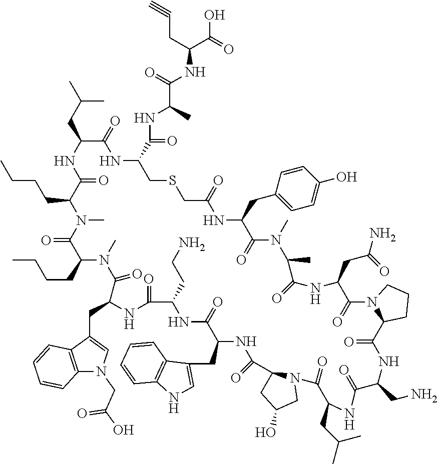 [SMILES: C#CC[C@H](NC(=O)[C@@H](C)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1020 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 31.1 mg, and its estimated purity by LCMS analysis was 98.1%.
Analysis condition B: Retention time=1.63 min; ESI-MS(+) m/z [M+H]⁺: 1997.3.

### Preparation of INT-1021

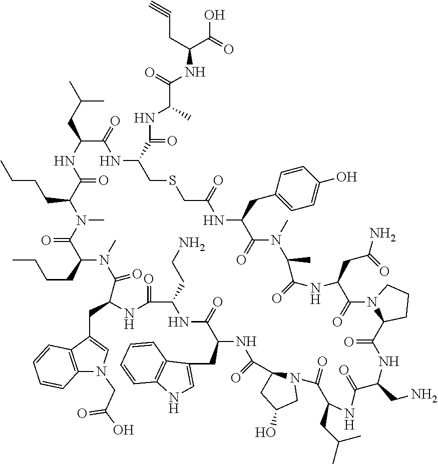 [SMILES: C#CC[C@H](NC(=O)[C@H](C)NC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1021 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 12 mg, and its estimated purity by LCMS analysis was 100%.
Analysis condition B: Retention time=1.62 min; ESI-MS(+) m/z [M+2H]²⁺: 999.3.

### Preparation of INT-1022

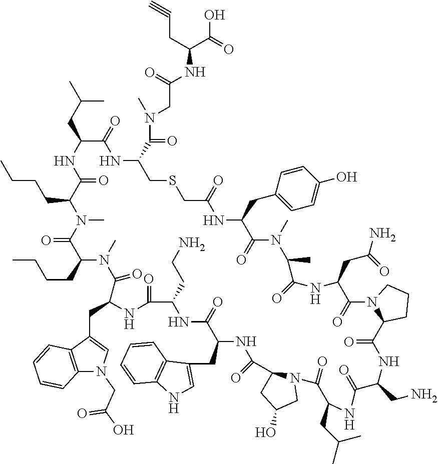 [SMILES: C#CC[C@H](NC(=O)CN(C)C(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

INT-1022 was prepared, using chlorotrityl resin preloaded with Fmoc-Pra-OH, on a 50 μmol scale, following the general synthetic sequence described for the preparation of INT-1001. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 1⁸-58% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 11 mg, and its estimated purity by LCMS analysis was 98.3%.
Analysis condition A: Retention time=1.59 min; ESI-MS(+) m/z [M+H]⁺: 1996.9.

### Preparation of INT-1023

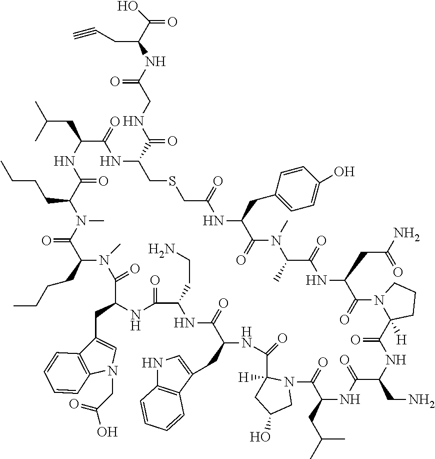 [SMILES: C#CC[C@H](NC(=O)CNC(=O)[C@@H]1CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N(C)[C@@H](CCCC)C(=O)N(C)[C@@H](CCCC)C(=O)N[C@@H](CC(C)C)C(=O)N1)C(=O)O]

The linear sequence of INT-1023 was prepared, using chlorotrityl resin preloaded with Fmoc-Pra-OH, according to the synthetic sequence described previously (see Scheme 1). The linear sequence Resin A in Scheme 1) was cleavaged and globally deprotected using “Global Deprotection Method A” and subsequently cyclized using “Cyclization Method A”. The crude was purified by reverse phase HPLC. The yield of the product was 30.4 mg, and its estimated purity by LCMS analysis was 95.1%.
Analysis condition A: Retention time=1.53 min; ESI-MS(+) m/z [M+H]⁺: 1983.8.

### Final Compound Synthesis

The following is a detailed example on final compounds synthesized via a solution phase click reaction.

### Example 1001

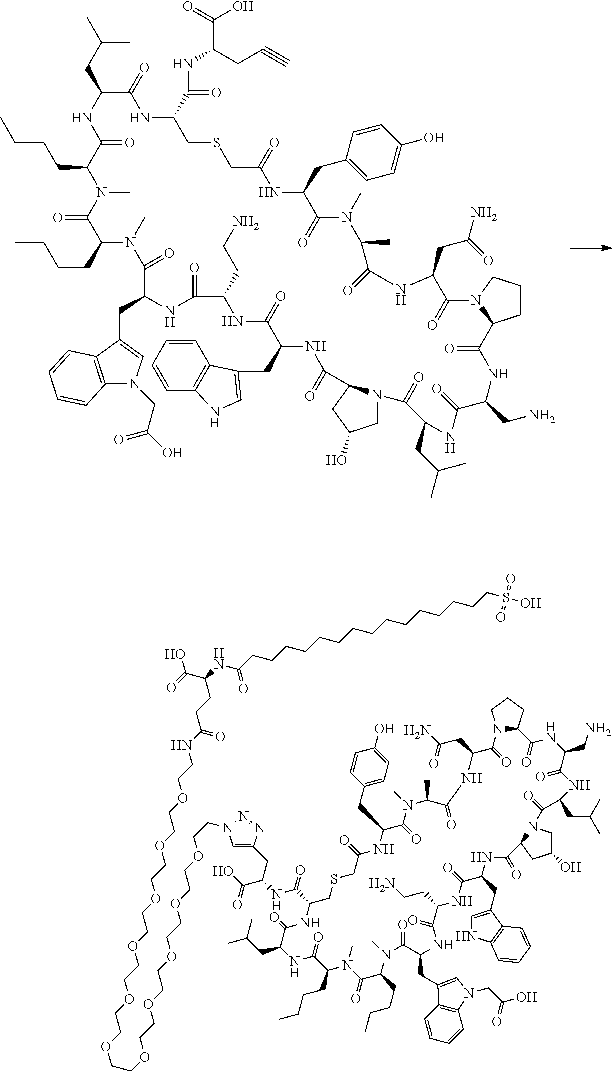

The compound was synthesized following the general procedure “Solution Phase Click Method A”. To a 20-ml scintillation vial is added 100-fold the needed amount of sodium (R)-2-((S)-1,2-dihydroxyethyl)-4-hydroxy-5-oxo-2,5-dihydrofuran-3-olate (0.987 mg, 4.89 μmol) and copper(II) sulfate pentahydrate (0.622 mg, 2.492 μmol). The reaction was diluted with water (10 mL). This solution was shaken at RT for 1-10 min. The resulting yellowish slurry was added to the reaction.

To the vial containing INT-1001 (24 mg, 12.0 μmol) was added tBuOH/water (v/v 1:1, 1 mL) and (S)-1-azido-37-oxo-40-(16-sulfohexadecanamido)-3,6,9,12,15,18,21,24,27,30,33-undecaoxa-36-azahentetracontan-41-oic acid (N3-Peg11-γGlu-FSA16, 16.49 mg, 0.016 mmol), followed by 100 μL of the above copper solution. The mixture was shaken at rt for 1 h and the progress was monitored by LC/MS. After completion, the mixture was diluted with CH₃CN:aq. ammonium bicarbonate solution (v/v 1:1), filtered, and submitted to single compound purification.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 26% B, 26-66% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 8.8 mg, and its estimated purity by LCMS analysis was 99%.
Analysis condition A: Retention time=1.55 min; ESI-MS(+) m/z [M+3H]³⁺: 982.10.
Analysis condition B: Retention time=1.87 min; ESI-MS(+) m/z [M+3H]³⁺: 981.98.

Following similar procedures as Example 1001, the following compounds in Examples 1002-1006 and 1009 were obtained.

### Preparation of Example 1002

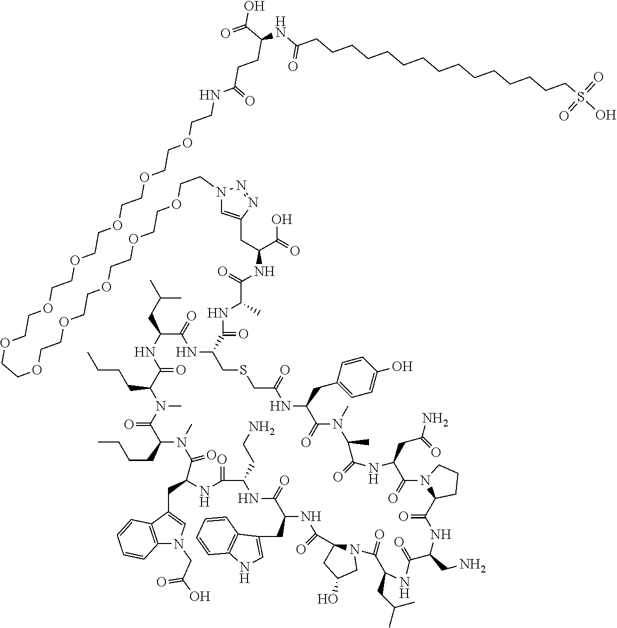 [SMILES: CCCC[C@H]1C(=O)N(C)[C@@H](CCCC)C(=O)N[C@@H](CC(C)C)C(=O)N[C@H](C(=O)N[C@@H](C)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Example 1002 was prepared on a 12 μmol scale. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 5.6 mg, and its estimated purity by LCMS analysis was 97.8%. Analysis condition 2: Retention time=1.6 min; ESI-MS(+) m/z²⁺: 1006.2.

### Preparation of Example 1003

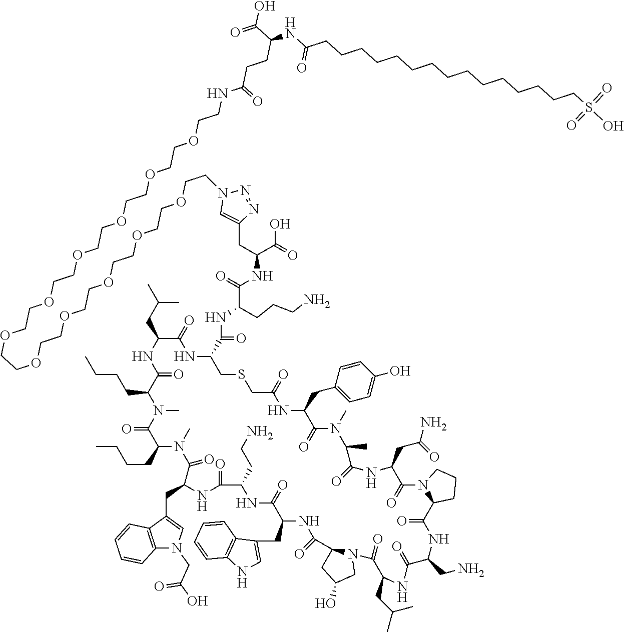 [SMILES: CCCC[C@H]1C(=O)N(C)[C@@H](CCCC)C(=O)N[C@@H](CC(C)C)C(=O)N[C@H](C(=O)N[C@@H](CCCN)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Example 1003 was prepared, on a 10.3 μmol scale. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 22% B, 22-62% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 7 mg, and its estimated purity by LCMS analysis was 91.1%. Analysis condition 1: Retention time=1.54 min; ESI-MS(+) m/z²⁺: 1530.1.

### Preparation of Example 1004

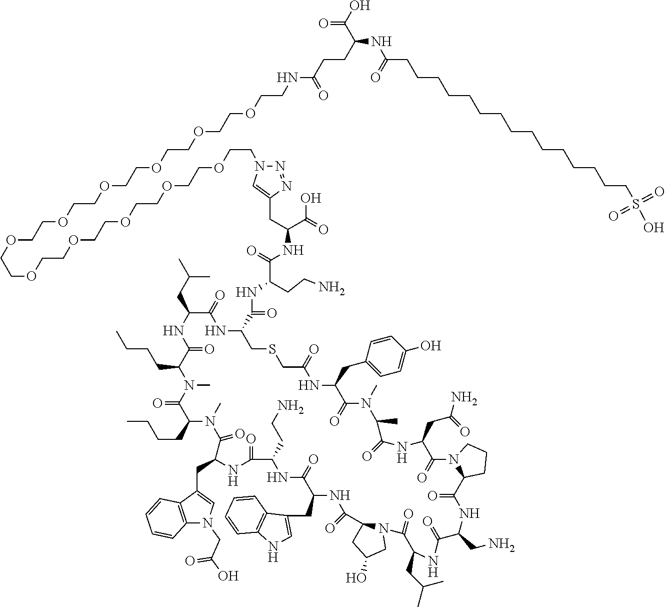 [SMILES: CCCC[C@H]1C(=O)N(C)[C@@H](CCCC)C(=O)N[C@@H](CC(C)C)C(=O)N[C@H](C(=O)N[C@@H](CCN)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Example 1004 was prepared, on a 9.9 μmol scale. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 3.7 mg, and its estimated purity by LCMS analysis was 91.8%. Analysis condition 2: Retention time=1.76 min; ESI-MS(+) m/z²⁺: 1015.3.

### Preparation of Example 1005

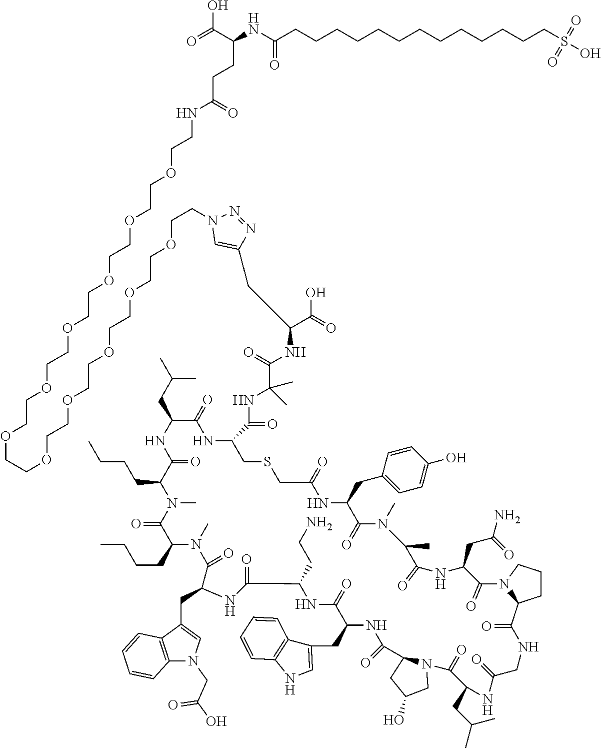 [SMILES: CCCC[C@H]1C(=O)N(C)[C@@H](CCCC)C(=O)N[C@@H](CC(C)C)C(=O)N[C@H](C(=O)NC(C)(C)C(=O)N[C@@H](Cc2cn(CCOCCOCCOCCOCCOCCOCCOCCOCCOCCOCCOCCNC(=O)CC[C@H](NC(=O)CCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)NCC(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Example 1005 was prepared, on a 8 μmol scale. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 6.7 mg, and its estimated purity by LCMS analysis was 98.9%. Analysis condition 1: Retention time=1.54 min; ESI-MS(+) m/z²ⁿ: 1515.1.

### Preparation of Example 1006

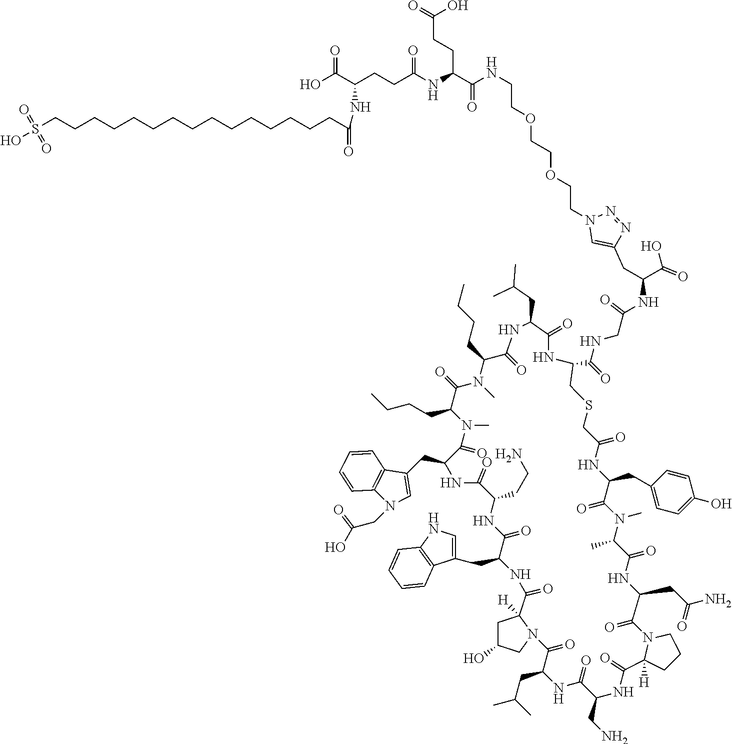 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCNC(=O)[C@H](CCC(=O)O)NC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Example 1006 was prepared on a 10.6 mmol scale. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 1.5 mg, and its estimated purity by LCMS analysis was 91%.
Analysis condition A: Retention time=1.49 min; ESI-MS(+) m/z [M+2H]²⁺: 1367.3.

### Preparation of Example 1007

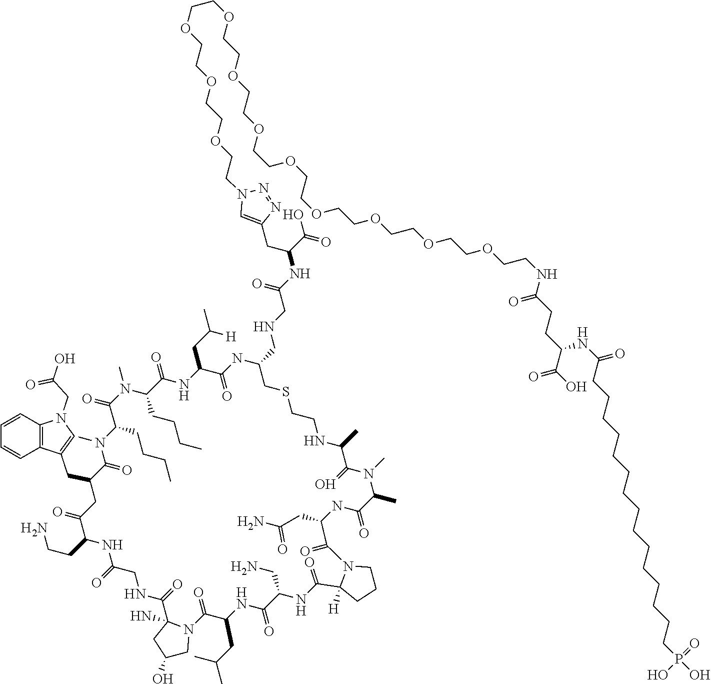

The linear alkyne intermediate material of INT-1023 (299 mg, 100 moles) was subjected to “Click Reaction On Resin Method A” with N₃-Peg11-γGlu-FPA16, followed by “Global Deprotection Method A” and “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 2.8 mg, and its estimated purity by LCMS analysis was 100%.
Analysis condition A: Retention time=1.75 min; ESI-MS(+) m/z [M+3H]³⁺: 1001.3.
Analysis condition B: Retention time=1.82 min; ESI-MS(+) m/z [M+3H]³⁺: 1001.3.

### Preparation of Example 1008

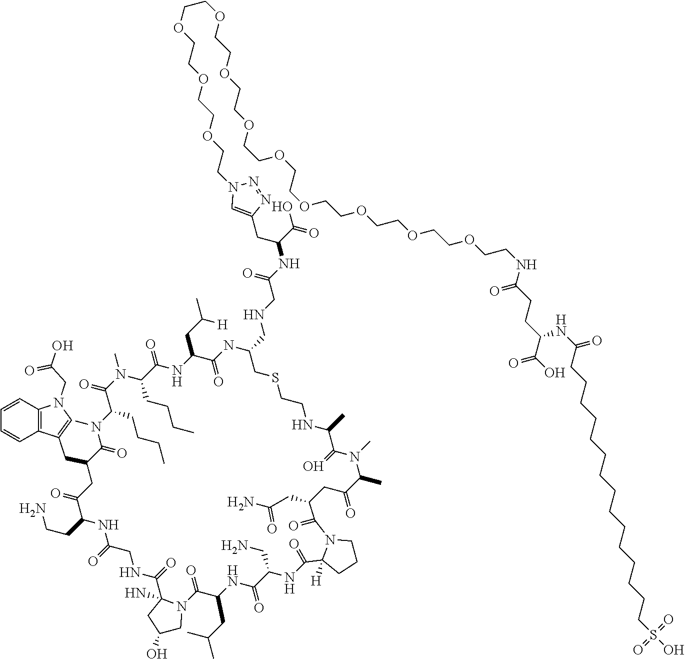

Example 1008 was prepared on a 100 μmol scale following the procedure as Example 1007 starting from the linear sequence of INT-1023 using “Click Reaction On Resin Method A” with N₃-Peg11-□Glu-FSA16, followed by “Global Deprotection Method A” and “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 24% B, 24-64% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 3 mg, and its estimated purity by LCMS analysis was 98.2%. Analysis condition B: Retention time=1.83 min; ESI-MS(+) m/z [M+3H]³⁺: 1001.08.

### Preparation of Example 1009

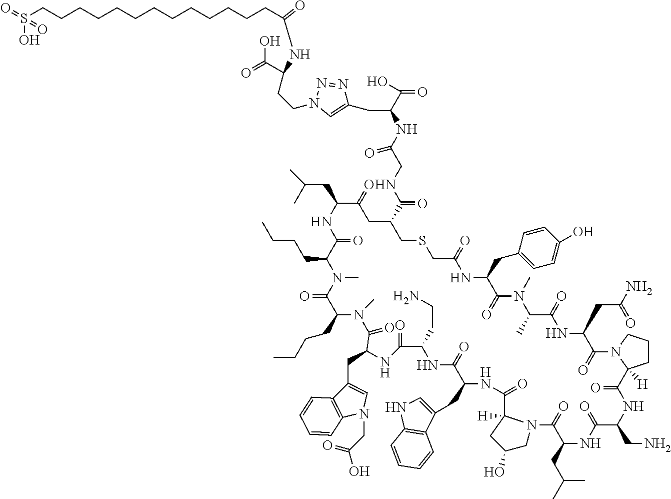 [SMILES: CCCC[C@H]1C(=O)N(C)[C@@H](CCCC)C(=O)N[C@@H](CC(C)C)C(=O)C[C@H](C(=O)NCC(=O)N[C@@H](Cc2cn(CC[C@H](NC(=O)CCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Example 1009 was prepared on a 50 μmol scale following the procedures described in Example 1007 starting from the linear sequence of INT-1023 using “Click Reaction On-Resin Method A” with (S)-4-azido-2-(14-sulfotetradecanamido)butanoic acid, followed by “Global Deprotection Method A” and “Cyclization Method A”.

Click chemistry was conducted on resin-bound capped linear peptide A on a 0.05 mmol scale: The resin A in a Bio Rad tube was washed with DCM (3×5 mL) and then with DMF (4×5 mL). To the resin was added the following solution: bis(2,2,6,6-tetramethyl-3,5-heptanedionato)copper(II) (10.75 mg, 0.025 mmol), vitamin C (0.026 g, 0.150 mmol), 2,6-lutidine (0.058 ml, 0.500 mmol), DIEA (0.087 ml, 0.500 mmol), THE (1.500 mL) and DMF (1.5 mL). The above solution was added to a weighed vial of (S)-4-azido-2-(14-sulfotetradecanamido)butanoic acid (0.037 g, 0.085 mmol). The resulting solution was added to the resin. The tube was capped and shaken overnight on a shaker table. The solution was drained and the resin was washed with DMF (6×5 ml), then DCM (3×5 mL). The resin was treated with 95% TFA 2.5% TIS and 2.5% DTT (5 mL) and shaken at rt for 1 h. The solution was drained to a 50-mL Falcon tube, which contained 35 mL of cooled Et₂O. The resulting white precipitate was collected by centrifuge (3×4 min×300 RPM), discarding the ether layers. The pellet was air-dried for 1 hour at RT. It was dissolved in DMF (30 m) and DIEA (1 mL) was added. The reaction mixture was shaken at rt for 6 h. The reaction was concentrated on a Genevac. The resulting sample was dissolved in 2 ml DMF and submitted to purification.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-60% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 4.2 mg, and its estimated purity by LCMS analysis was 89%. Analytical LC/MS was used to determine the final purity. Injection 1 conditions: Column: Waters XBridge C18, 2.1 mm×50 mm, 1.7 m particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results (analysis condition A): Purity: 88.8%; Observed Mass: ESI-MS(+) m/z [M+2H]²+1210.10; Retention Time: 1.5 min. Injection 2 conditions: Column: Waters XBridge C18, 2.1 mm×50 mm, 1.7 m particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results (analysis condition B): Purity: 98.1%; Observed Mass: ESI-MS(+) m/z [M+2H]²⁺ 1209.80; Retention Time: 1.74 min.

### Preparation of Example 1010

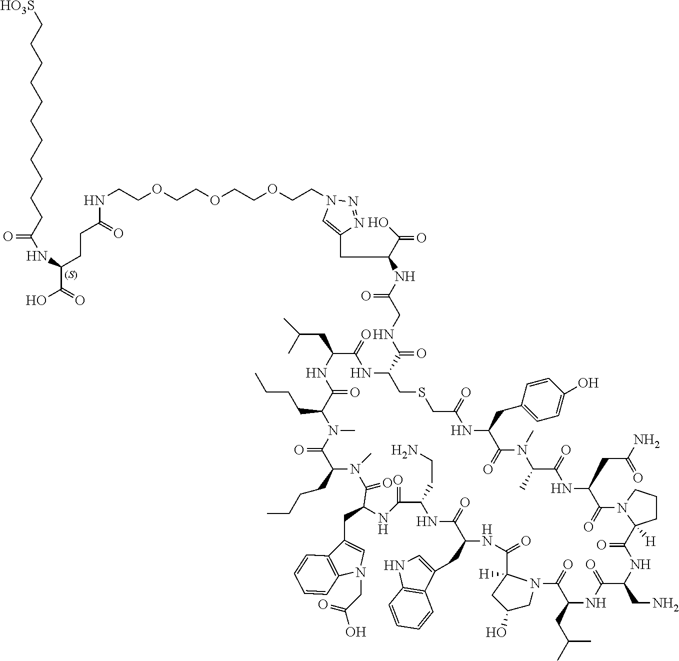

Example 1010 was prepared on a 44 μmol scale following the procedure of Example 1001 using “Click Reaction On-Resin Method A” to react INT-1023 with N₃-Peg3-γGlu-FSA12.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 17.0 mg, and its estimated purity by LCMS analysis was 98%. Analytical LC/MS was used to determine the final purity. Injection 1 conditions: Column: Waters XBridge C18, 2.1 mm×50 mm, 1.7 m particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection

1 (Analysis condition A) results: Purity: 99.2%; Observed Mass: 865.80; Retention Time: 1.46 min. Injection 2 conditions: Column: Waters XBridge C18, 2.1 mm×50 mm, 1.7 m particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 (Analysis condition B) results: Purity: 97.7%; Observed Mass: 1297.60; Retention Time: 1.66 min.

### Methods for Testing the Ability of Macrocyclic Peptides to Compete for the Binding of PD-1 to PD-L1 Using Homogenous Time-Resolved Fluorescence (HTRF) Binding Assays

The ability of the macrocyclic peptides of the present disclosure to bind to PD-L1 was investigated using a PD-1/PD-L1 Homogenous Time-Resolved Fluorescence (HTRF) binding assay.

### Methods

Homogenous Time-Resolved Fluorescence (HTRF) Assays of Binding of Soluble PD-1 to Soluble PD-L1. Soluble PD-1 and soluble PD-L1 refers to proteins with carboxyl-end truncations that remove the transmembrane-spanning regions and are fused to heterologous sequences, specifically the Fc portion of the human immunoglobulin G sequence (Ig) or the hexahistidine epitope tag (His). All binding studies were performed in an HTRF assay buffer consisting of dPBS supplemented with 0.1% (w/v) bovine serum albumin and 0.05% (v/v) Tween-20. For the PD-1-Ig/PD-L1-His binding assay, inhibitors were pre-incubated with PD-L1-His (10 nM final) for 15 m in 4 μl of assay buffer, followed by addition of PD-1-Ig (20 nM final) in 1 μl of assay buffer and further incubation for 15 m. PD-L1 fusion proteins from either human, cynomologous macaques, mouse, or other species were used. HTRF detection was achieved using europium crypate-labeled anti-Ig monoclonal antibody (1 nM final) and allophycocyanin (APC) labeled anti-His monoclonal antibody (20 nM final). Antibodies were diluted in HTRF detection buffer and 5 μl was dispensed on top of binding reaction. The reaction was allowed to equilibrate for 30 minutes and signal (665 nm/620 nm ratio) was obtained using an EnVision fluorometer. Additional binding assays were established between PD-1-Ig/PD-L2-His (20 and 5 nM, respectively), CD80-His/PD-L1-Ig (100 and 10 nM, respectively) and CD80-His/CTLA4-Ig (10 and 5 nM, respectively).

Binding/competition studies between biotinylated Compound No. 71 (See Example 72 on Page 212 in WO2014/151634 for structure) and human PD-L1-His were performed as follows. Macrocyclic peptide inhibitors were pre-incubated with PD-L1-His (10 nM final) for 60 minutes in 4 μl of assay buffer followed by addition of biotinylated Compound No. 71 (0.5 nM final) in 1 μl of assay buffer. Binding was allowed to equilibrate for 30 minutes followed by addition of europium crypated labeled Streptavidin (2.5 pM final) and APC-labeled anti-His (20 nM final) in 5 μl of HTRF buffer. The reaction was allowed to equilibrate for 30 m and signal (665 nm/620 nm ratio) was obtained using an EnVision fluorometer.

Recombinant Proteins. Carboxyl-truncated human PD-1 (amino acids 25-167) with a C-terminal human Ig epitope tag [hPD-1 (25-167)-3S-IG] and human PD-L1 (amino acids 18-239) with a C-terminal His epitope tag [hPD-L1(19-239)-tobacco vein mottling virus protease cleavage site (TVMV)-His] were expressed in HEK293T cells and purified sequentially by recombinant Protein A affinity chromatography and size exclusion chromatography. Human PD-L2-His (Sino Biologicals), CD80-His (Sino Biologicals), CTLA4-Ig (RnD Systems) were all obtained through commercial sources.

Sequence of Recombinant Human PD-1-Ig

| hPD1(25-167)-3S-IG | |
|---|---|
| (SEQ ID NO: 1) | |
| 1 | LDSPDRPWNP PTFSPALLVV TEGDNATFTC SFSNTSESFV LNWYRMSPSN |
| 51 | QTDKLAAFPE DRSQPGQDCR FRVTQLPNGR DFHMSVVRAR RNDSGTYLCG |
| 101 | AISLAPKAQI KESLRAELRV TERRAEVPTA HPSPSPRPAG QFQGSPGGGG |
| 151 | GREPKSSDKT HTSPPSPAPE LLGGSSVFLF PPKPKDTLMI SRTPEVTCVV |
| 201 | VDVSHEDPEV KFNWYVDGVE VHNAKTKPRE EQYNSTYRVV SVLTVLHQDW |
| 251 | LNGKEYKCKV SNKALPAPIE KTISKAKGQP REPQVYTLPP SRDELTKNQV |
| 301 | SLTCLVKGFY PSDIAVEWES NGQPENNYKT TPPVLDSDGS FFLYSKLTVD |
| 351 | KSRWQQGNVF SCSVMHEALH NHYTQKSLSL SPGK |

Sequence of Recombinant Human PD-L1-TVMV-His (PD-L1-His)

| hPDL1(19-239)-TVMV-His | |
|---|---|
| (SEQ ID NO: 2) | |
| 1 | FTVTVPKDLY VVEYGSNMTI ECKFPVEKQL DLAALIVYWE MEDKNIIQFV |
| 51 | HGEEDLKVQH SSYRQRARLL KDQLSLGNAA LQITDVKLQD AGVYRCMISY |
| 101 | GGADYKRITV KVNAPYNKIN QRILVVDPVT SEHELTCQAE GYPKAEVIWT |
| 151 | SSDHQVLSGK TTTTNSKREE KLFNVTSTLR INTTTNEIFY CTFRRLDPEE |
| 201 | NHTAELVIPE LPLAHPPNER TGSSETVRFQ GHHHHHH |

293T-hPD-L1 Cell Binding High-Content Screening Assay (CBA). Phycoerythrin (PE) was covalently linked to the Ig epitope tag of human PD-1-Ig and fluorescently-labeled PD-1-Ig was used for binding studies with a human embryonic kidney cell line (293T) stably over-expressing human PD-L1 (293T-hPD-L1). Briefly, 2×10³ 293T-hPD-L1 cells were seeded into 384 well plates in 20 μl of DMEM supplemented with 10% fetal calf serum and cultured overnight. 125 nl of compound was added to cells followed by 5 μl of PE-labeled PD-1-Ig (0.5 nM final), diluted in DMEM supplemented with 10% fetal calf serum, followed by incubation at 37° C. for Ih. Cells were washed 3× in 100 μl dPBS followed by fixation with 30 μl of 4% paraformaldehyde in dPBS containing 10 μg/ml Hoechst 33342 for 30 min at room temperature. Cells were washed 3× in 100 μl of dPBS followed by final addition of 15 μl of dPBS. Data was collected and processed using a Cell Insight NXT High Content Imager and associated software.

Table 1 lists the IC₅₀ values for representative examples of this disclosure measured in the PD-1/PD-L1 Homogenous Time-Resolved Fluorescence (HTRF) binding assay and the 293T-hPD-L1 Cell Binding High-Content Screening Assay.

TABLE 1

| Example | LE HTRF hPD1 hPDL1 | 293T-hPD-L1_PD-1 |
|---|---|---|
| Number | IC₅₀ (μM) | IC50 (μM) |
| 1001 | 0.0006 | 0.0000 |
| 1002 | 0.0020 | 0.0001 |
| 1003 | 0.0381 | 0.0028 |
| 1004 | NT | NT |
| 1005 | 0.0102 | 0.0011 |
| 1006 | NT | 0.0618 |
| 1007 | 0.0140 | 0.0016 |
| 1008 | 0.0073 | 0.0012 |
| 1009 | NT | NT |
| 1010 | NT | 0.00013 |

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

The compounds of formula (I) possess activity as inhibitors of the PD-1/PD-L1 interaction, and therefore, can be used in the treatment of diseases or deficiencies associated with the PD-1/PD-L1 interaction. Via inhibition of the PD-1/PD-L1 interaction, the compounds of the present disclosure can be employed to treat infectious diseases such as HIV, septic shock, Hepatitis A, B, C, or D and cancer.

It is to be appreciated that the Detailed Description section, and not the Summary and Abstract sections, is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

We claim:

1. A compound of formula (I) 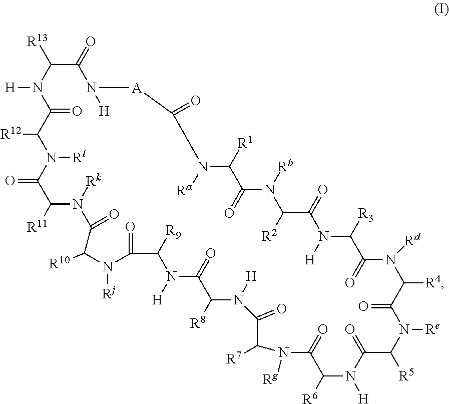
or a pharmaceutically acceptable salt thereof, wherein:
A is selected from 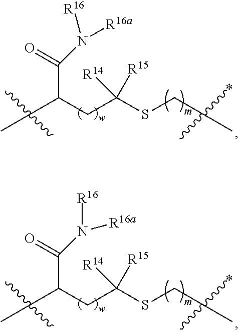
wherein: [symbol]
denotes the point of attachment to the carbonyl group and [symbol] denotes the point of attachment to the nitrogen atom;
n is 0 or 1;
m is 1 or 2;
u is 0 or 1;
w is 0, 1, or 2;
R^{x} is selected from hydrogen, amino, hydroxy, and methyl;
R¹⁴ and R¹¹ are independently selected from hydrogen and methyl;
R^{16a} is selected from hydrogen and C₁-C₆ alkyl;
R¹⁶ is selected from
—(C(R^{17a})₂)₂—X—R³⁰, —(C(R^{17a}R¹⁷))₀₋₂—X′—R³⁰, —(C(R^{17a}R¹⁷)₁₋₂C(O)NR^{16a})_{m′}—X′—R³⁰,
—C(R^{17a})₂C(O)N(R^{16a})C(R^{17a})₂—X′—R³¹, —(C(R^{17a}R¹⁷))₁₂C(O)N(R^{16a})C(R^{17a})₂—X′—R³¹,
—C(R^{17a})₂[C(O)N(R^{16a})C(R^{17a})₂]_{w′}—X—R³¹, —C(R^{17a}R¹⁷)₁₋₂[C(O)N(R^{16a})C(R^{17a}R¹⁷)₁₋₂]_{w′}—X′—R³¹,
—(C(R^{17a})(R¹⁷)C(O)NR^{16a})_{n′}—H; and
—(C(R^{17a})(R¹⁷)C(O)NR^{16a})_{m′}—C(R^{17a})(R¹⁷)—CO₂H;
wherein a PEG_{q′} spacer can be inserted in any part of the R¹⁶ (q′ is the number of —(CH₂CH₂O)— unit in a PEG spacer);
wherein w′ is 2 or 3;
n′ is 1-6;
m′ is 0-5;
q′ is 1-20
X is a chain of between 1 and 172 atoms wherein the atoms are selected from n:
carbon and oxygen and wherein the chain may contain one, two, three, or four groups selected from —NHC(O)—, —NHC(O)NH—, and —C(O)NH— embedded therein; and wherein the chain is optionally substituted with one to six groups independently selected from —CO₂H, —C(O)NH₂, —CH₂C(O)NH₂, and —(CH₂)₁₋₂CO₂H;
X′ is a chain of between 1 and 172 atoms wherein the atoms are selected from carbon and oxygen and wherein the chain may contain one, two, three, or four groups selected from —NHC(O)—, —NHC(O)NH—, and —C(O)NH— embedded therein; and wherein the chain is optionally substituted with one to six groups independently selected from —CO₂H, —C(O)NH₂, and —(CH₂)₁₋₂CO₂H, provided that X′ is other than unsubstituted PEG;
R³⁰ is selected from —SO₃H, —S(O)OH, and —P(O)(OH)₂;
R³¹ is selected from —S(O)₂OH, —S(O)OH, and —P(O)(OH)₂;
each R^{17a} is independently selected from hydrogen, C₁-C₆alkyl, —CH₂OH, —CH₂CO₂H, —(CH₂)₂CO₂H,
each R¹⁷ is independently selected from hydrogen, —CH₃, (CH₂)_{z}N₃,
—(CH₂)_{z}NH₂, —X—R³¹, —(CH₂)_{z}CO₂H, —CH₂OH, CH₂C≡CH, and —(CH₂)_{z}-triazolyl-X—R³⁵, wherein z is 1-6 and R³⁵ is selected from —SO₃H, —S(O)OH, and —P(O)(OH)₂; provided at least one R¹⁷ is other than hydrogen, —CH₃, or —CH₂OH;
provided at least one of R³⁰, R³¹, or R³⁵ is present;
R^{a}, R^{e}, R^{j}, and R^{k}, are each independently selected from hydrogen and methyl;
R¹, R², R³, R⁴, R⁵, R⁶, R⁷, R⁸, R⁹, R¹⁰, R¹¹, R¹², and R¹³ are independently selected from a natural amino acid side chain and an unnatural amino acid side chain or form a ring with the corresponding vicinal R group as described below;
R^{b} is methyl or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{d} is hydrogen or methyl, or, R^{d} and R⁴, together with the atoms to which they are attached, can form a ring selected from azetidine, pyrrolidine, morpholine, piperidine, piperazine, and tetrahydrothiazole; wherein each ring is optionally substituted with one to four groups independently selected from amino, cyano, methyl, halo, hydroxy, and phenyl;
R⁹ is hydrogen or methyl or R⁹ and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R¹ is methyl or, R¹ and R¹², together with the atoms to which they are attached, form a ring selected from azetidine and pyrrolidine, wherein each ring is optionally substituted with one to four independently selected from amino, cyano, methyl, halo, and hydroxy.

2. The compound according to claim 1, or a pharmaceutically acceptable salt thereof,
wherein A is 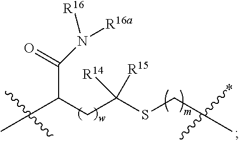
m is 1 and w is 0; and
R¹⁴, R¹⁵, and R^{16a} are each hydrogen.

3. The compound according to claim 1, or a pharmaceutically acceptable salt thereof, wherein
R¹⁶ is selected from —(C(R^{17a})₂)₂—X—R³⁰, —(C(R^{17a}R¹⁷))₀₋₂—X′—R³ and —(C(R^{17a}R¹⁷)₁₋₂C(O)NR^{16a})_{m′}X′—R³⁰,
each R^{17a} is selected from hydrogen, —CO₂H, and —(CH₂)₁₋₂CO₂H;
X is a chain of between 8 and 46 atoms wherein the atoms are selected from carbon and oxygen and wherein the chain may contain one, two, or three —NHC(O)—, C(O)NH groups embedded therein; and wherein the chain is optionally substituted with one or two groups independently selected from —CO₂H, —C(O)NH₂, —CH₂C(O)NH₂, and —CH₂CO₂H; and
R³⁰ is selected from —SO₃H and —P(O)(OH)₂.

4. The compound according to claim 1, or a pharmaceutically acceptable salt thereof, wherein
A is 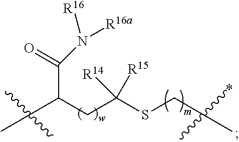
m is 1 and w is 0;
R¹⁴, R¹⁵, and R^{16a} are each hydrogen;
R¹⁶ is selected from —C(R^{17a})₂C(O)N(R^{16a})C(R^{17a})₂—X′—R³¹ and —(C(R^{17a}R¹⁷))₁₋₂C(O)N(R^{16a})C(R^{17a})₂—X′—R³¹,
each R^{17a} is selected from hydrogen, —CO₂H, and —CH₂CO₂H;
X′ is a chain of between 8 and 48 atoms wherein the atoms are selected from carbon and oxygen and wherein the chain may contain one, two, or three —NHC(O)— or —C(O)NH— groups embedded therein; and wherein the chain is optionally substituted with one or two groups independently selected from —CO₂H, —C(O)NH₂, —CH₂C(O)NH₂, and —CH₂CO₂H; provided that X′ is other than unsubstituted PEG; and
R³¹ is selected from —SO₃H and —P(O)(OH)₂.

5. The compound according to claim 1, or a pharmaceutically acceptable salt thereof, wherein A is 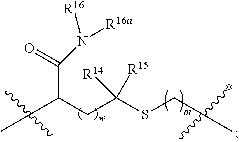
m is 1 and w is 0;
R¹⁴, R¹⁵, and R^{16a} are each hydrogen;
R¹⁶ is selected from —C(R^{17a})₂[C(O)N(R^{16a})C(R^{17a})_{z}]_{w′}—X—R³¹ and —C(R^{17a}R¹⁷)₁₋₂[C(O)N(R^{16a})C(R^{17a}R¹⁷)₁₋₂]_{w′}—X′—R³¹,
each R^{17a} is selected from hydrogen, —CO₂H, and —CH₂CO₂H;
X is a chain of between 8 and 48 atoms wherein the atoms are selected from carbon and oxygen and wherein the chain may contain one, two, or three —NHC(O)— or —C(O)NH— groups embedded therein; and wherein the chain is optionally substituted with one or two groups independently selected from —CO₂H, —C(O)NH₂, —CH₂C(O)NH₂, and —CH₂CO₂H; and
R³¹ is selected from —SO₃H and —P(O)(OH)₂.

6. The compound according to claim 1, or a pharmaceutically acceptable salt thereof, wherein A is 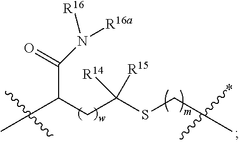
m is 1 and w is 0;
R¹⁴, R¹⁵, and R^{16a} are each hydrogen; and
R¹⁶ is —(C(R^{17a})(R¹⁷)C(O)NR^{16a})_{n′}—H.
each R^{17a} is hydrogen; and
each R¹⁷ is selected from hydrogen, —CH₃, (CH₂)_{z}N₃, —(CH₂)_{z}NH₂, —X—R³¹,
—(CH₂)_{z}CO₂H, —CH₂OH, CH₂C≡CH, and —(CH₂)_{z}-triazolyl-X—R³⁵; provided at least one R¹⁷ is other than hydrogen, —CH₃, or —CH₂OH, and provided at least one R³¹ or R³⁵ is present;
z is 1-4;
R³¹ is selected from —SO₃H and —P(O)(OH)₂;
X is a chain of between 7 and 155 atoms wherein the atoms are selected from carbon and oxygen and wherein the chain may contain one, two, or three —NHC(O)—, or —C(O)NH— groups embedded therein; and wherein the chain is optionally substituted with one or two groups independently selected from —CO₂H, —C(O)NH₂, —CH₂C(O)NH₂, and —CH₂CO₂H; and
R³⁵ is selected from —SO₃H and —P(O)(OH)₂.

7. The compound according to claim 1, or a pharmaceutically acceptable salt thereof, wherein
A is 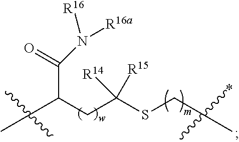
m is 1 and w is 0;
R¹⁴, R¹⁵, and R^{16a} are each hydrogen;
R¹⁶ is —(CR^{17a})(R¹⁷)C(O)NR^{16a})_{m′}—C(R^{17a})(R¹⁷)—CO₂H;
m′ is 0-3;
each R^{17a} is hydrogen;
each R¹⁷ is selected from hydrogen, —CH₃, (CH₂)_{z}N₃, —(CH₂)_{z}NH₂, —X—R³¹,
—(CH₂)_{z}CO₂H, —CH₂OH, CH₂C≡CH, —(CH₂)_{z}-triazolyl-X—R³⁵; and 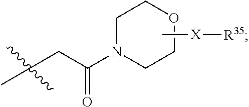
provided at least one R¹⁷ is other than hydrogen, —CH₃, or —CH₂OH, and provided at least one R³¹ or R³⁵ is present;
z is 1-4;
R³¹ is selected from —SO₃H and —P(O)(OH)₂;
X is a chain of between 10 and 60 atoms wherein the atoms are selected from carbon and oxygen and wherein the chain may contain one, two, or three —NHC(O)—, —C(O)NH— groups embedded therein; and wherein the chain is optionally substituted with one or two groups independently selected from —CO₂H, —C(O)NH₂, —CH₂C(O)NH₂, and —CH₂CO₂H; and
R³⁵ is selected from —SO₃H and —P(O)(OH)₂.

8. The compound according to claim 1, or a pharmaceutically acceptable salt thereof, wherein
R¹ is phenylC₁-C₃alkyl wherein the phenyl part is optionally substituted with hydroxyl, halo, or methoxy;
R² is C₁-C₇alkyl or, R² and R^{b}, together with the atoms to which they are attached, form a piperidine ring;
R³ is NR^{x}R^{y}(C₁-C₇alkyl), NR^{u}R^{v}carbonylC₁-C₃alkyl, or carboxyC₁-C₃alkyl;
R⁴ and R^{d}, together with the atoms to which they are attached, form a pyrrolidine ring;
R⁵ is hydroxyC₁-C₃alkyl, imidazolylC₁-C₃alkyl, or NR^{x}R^{y}(C₁-C₇alkyl);
R⁶ is carboxyC₁-C₃alkyl, NR^{u}R^{v}carbonylC₁-C₃alkyl, NR^{x}R^{y}(C₁-C₇alkyl), or C₁-C₇alkyl;
R⁷ and R⁹, together with the atoms to which they are attached, form a pyrrolidine ring optionally substituted with hydroxy;
R⁸ and R¹⁰ are benzothienyl or indolylC₁-C₃alkyl optionally substituted with carboxyC₁-C₃alkyl;
R⁹ is hydroxyC₁-C₃alkyl, aminoC₁-C₄alkyl, or C₁-C₇alkyl, R¹¹ is C₁-C₃alkoxyC₁-C₃alkyl or C₁-C₇alkyl;
R¹² is C₁-C₇alkyl or hydroxyC₁-C₃alkyl; and
R¹³ is C₁-C₇ alkyl, carboxyC₁-C₃alkyl, or —(CH₂)₃NHC(NH)NH₂.

9. The compound according to claim 1, or a pharmaceutically acceptable salt thereof, wherein
A is 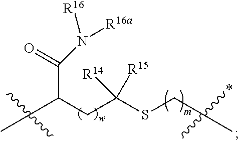
m is 1 and w is 0;
R¹⁴ and R¹⁵, are each hydrogen;
R^{16a} is hydrogen or methyl;
R^{d} is methyl or, R^{d} and R⁴, together with the atoms to which they are attached, form a ring selected from azetidine, pyrrolidine, morpholine, piperidine, piperazine, and tetrahydrothiazole; wherein each ring is optionally substituted with one or two groups independently selected from amino, cyano, methyl, halo, hydroxy, and phenyl;
R⁹ is methyl or, R⁹ and R⁷, together with the atoms to which they are attached,
form a ring selected from azetidine, pyrrolidine, morpholine, piperidine, piperazine, and tetrahydrothiazole; wherein each ring is optionally substituted with one or two groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and pyrrolidine.

10. A compound or a pharmaceutically acceptable salt thereof which is

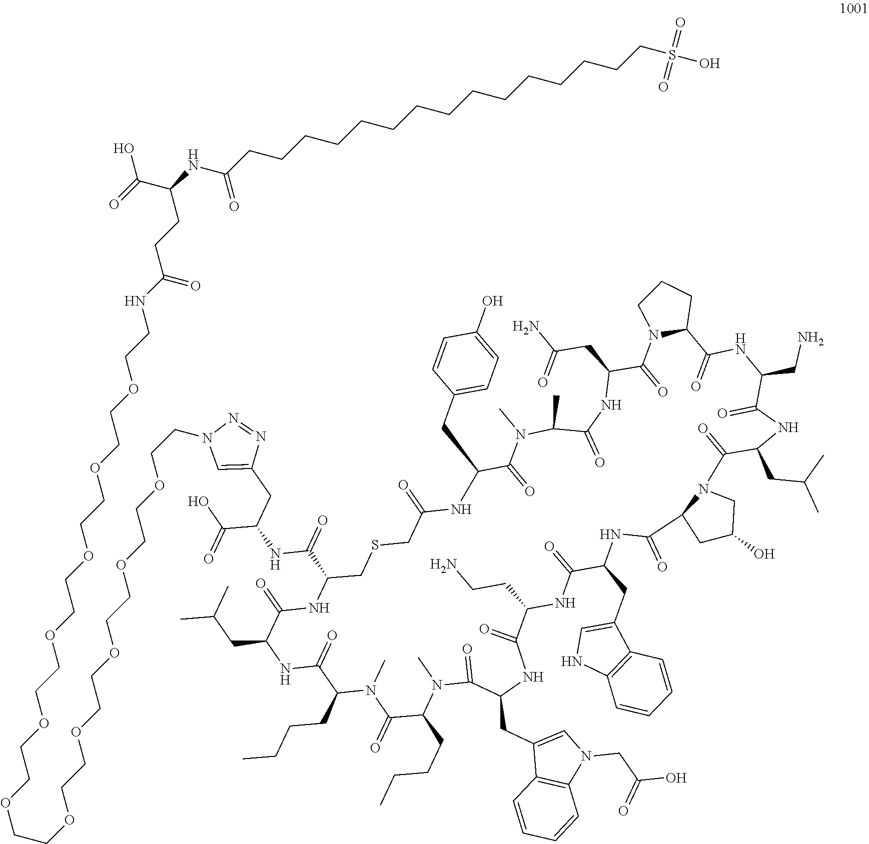 [SMILES: CCCC[C@H]1C(=O)N(C)[C@@H](CCCC)C(=O)N[C@@H](CC(C)C)C(=O)N[C@H](C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

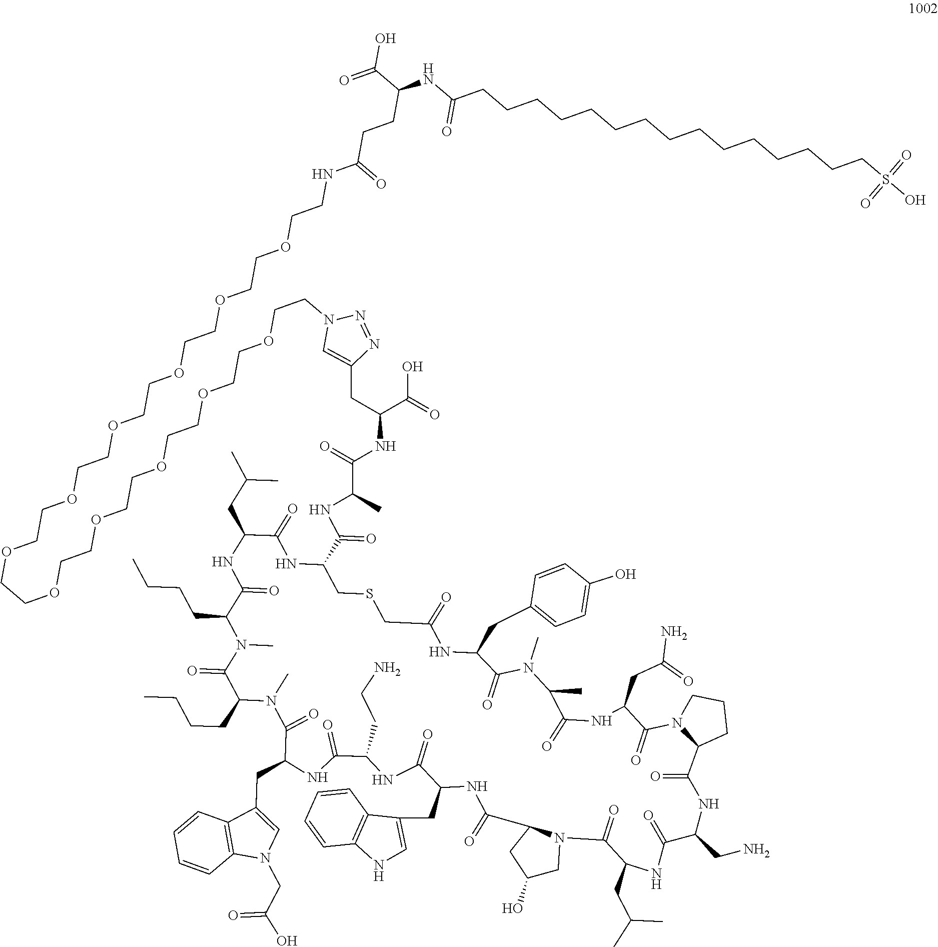 [SMILES: CCCC[C@H]1C(=O)N(C)[C@@H](CCCC)C(=O)N[C@@H](CC(C)C)C(=O)N[C@H](C(=O)N[C@H](C)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

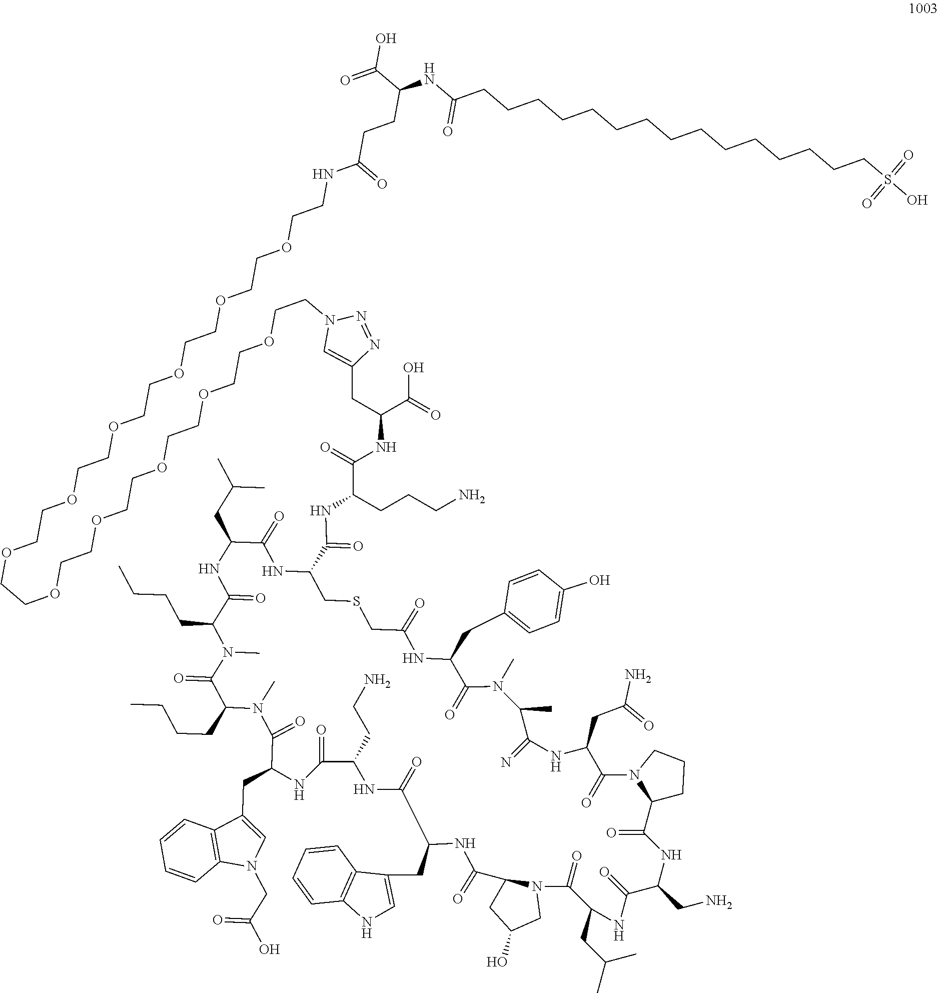 [SMILES: CCCC[C@H]1C(=O)N(C)[C@@H](CCCC)C(=O)N[C@@H](CC(C)C)C(=O)N[C@H](C(=O)N[C@@H](CCCN)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=N)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

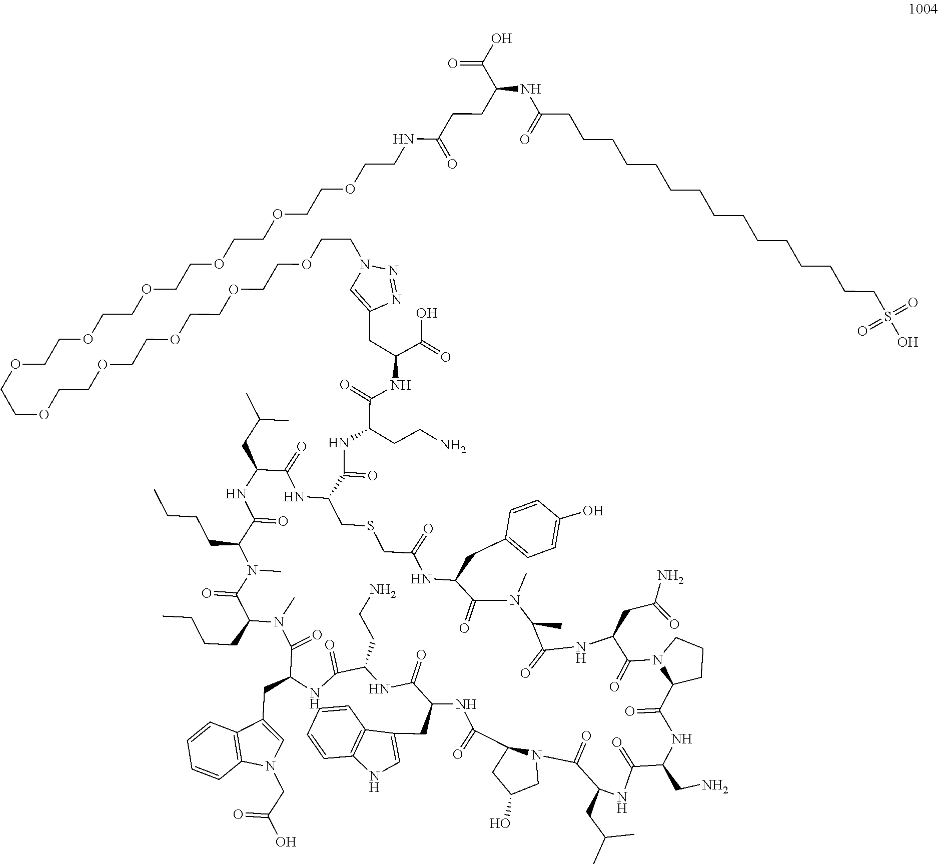 [SMILES: CCCC[C@H]1C(=O)N(C)[C@@H](CCCC)C(=O)N[C@@H](CC(C)C)C(=O)N[C@H](C(=O)N[C@@H](CCN)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

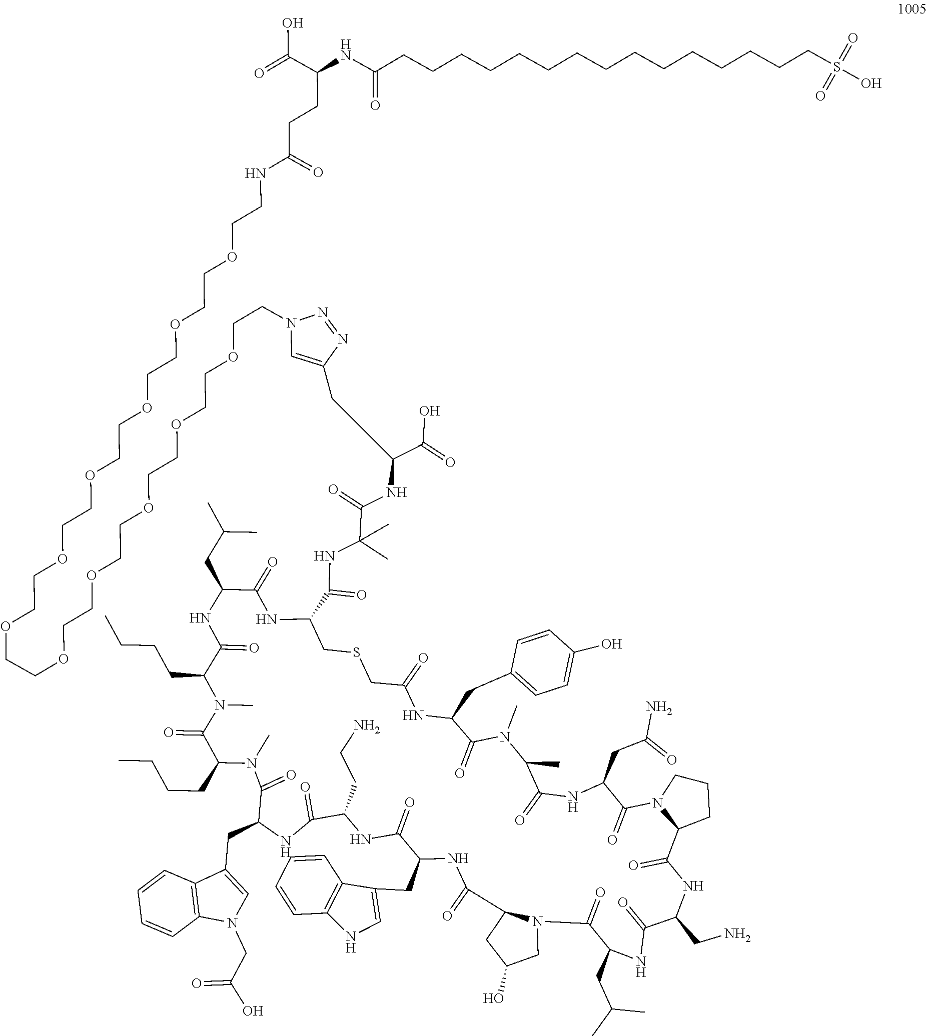 [SMILES: CCCC[C@H]1C(=O)N(C)[C@@H](CCCC)C(=O)N[C@@H](CC(C)C)C(=O)N[C@H](C(=O)NC(C)(C)C(=O)N[C@@H](Cc2cn(CCOCCOCCOCCOCCOCCOCCOCCOCCOCCOCCOCCNC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

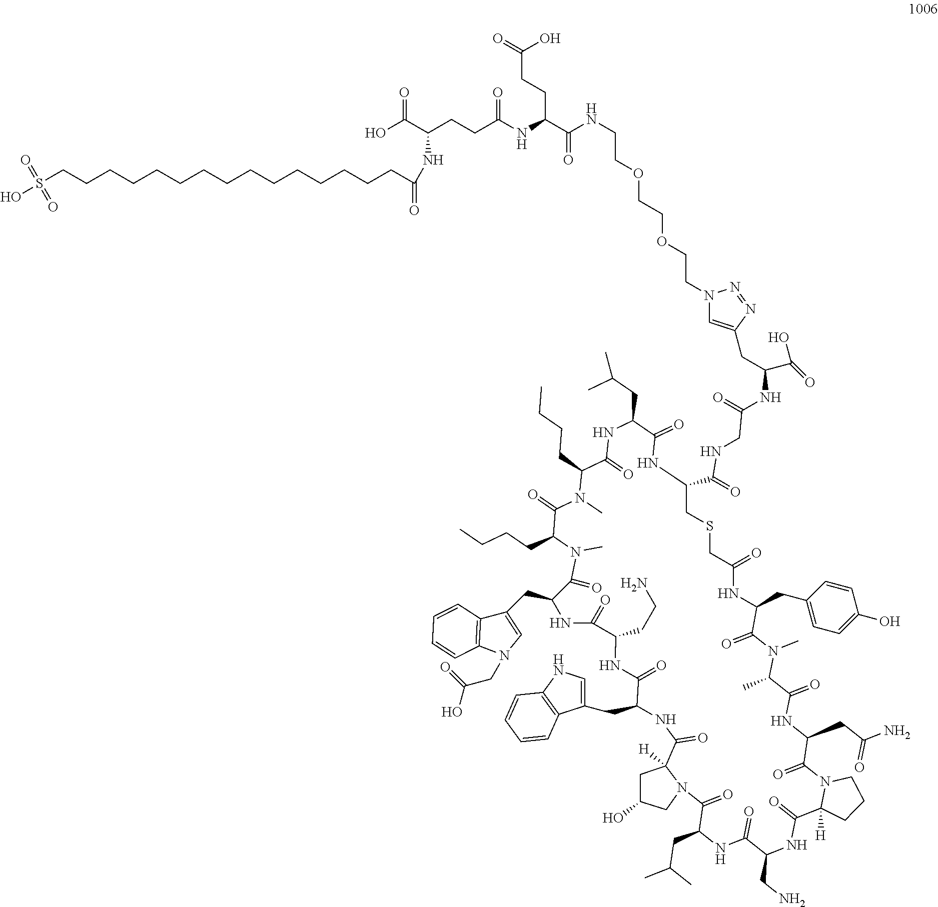 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCNC(=O)[C@H](CCC(=O)O)NC(=O)CC[C@H](NC(=O)CCCCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

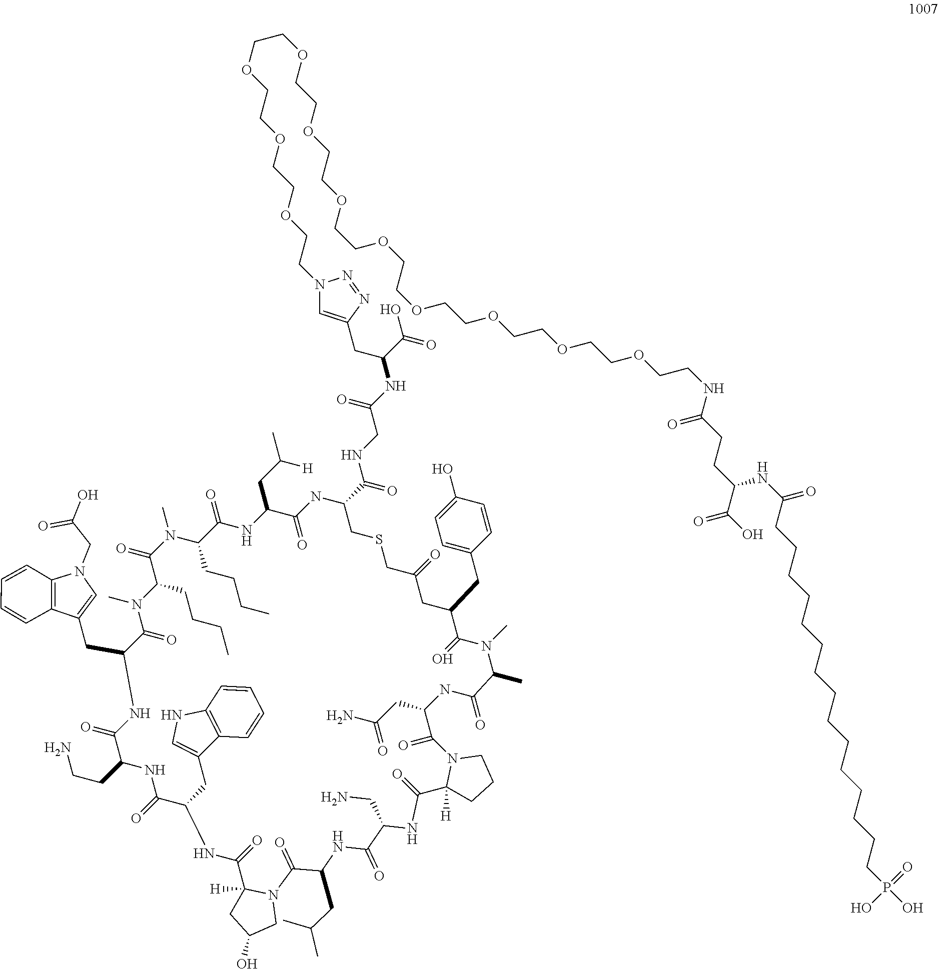 [SMILES: CCCC[C@H]1C(=O)N(C)[C@@H](CCCC)C(=O)N[C@@H](CCC)C(=O)N[C@H](C(=O)NCC(=O)N[C@@H](Cc2cn(CCOCCOCCOCCOCCOCCOCCOCCOCCOCCOCCOCCNC(=O)CC[C@H](NC(=O)CCCCCCCCCCCCCCCP(=O)(O)O)C(=O)O)nn2)C(=O)O)CSCC(=O)C[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

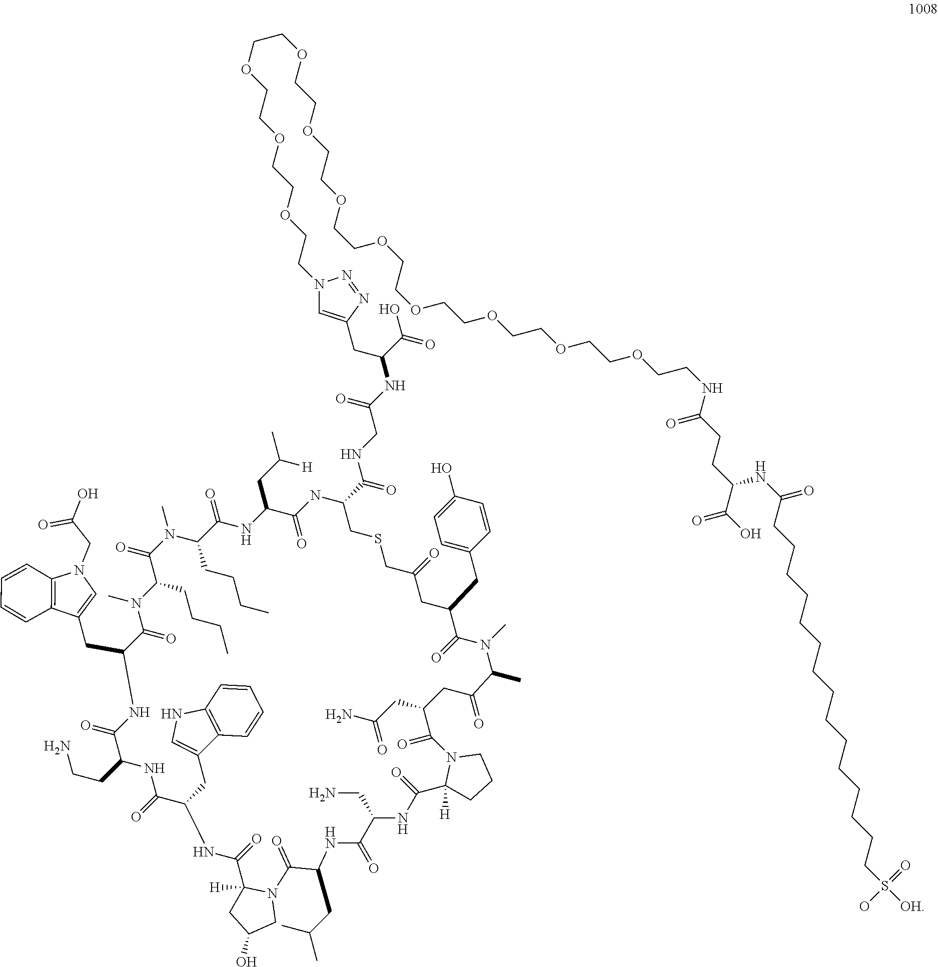

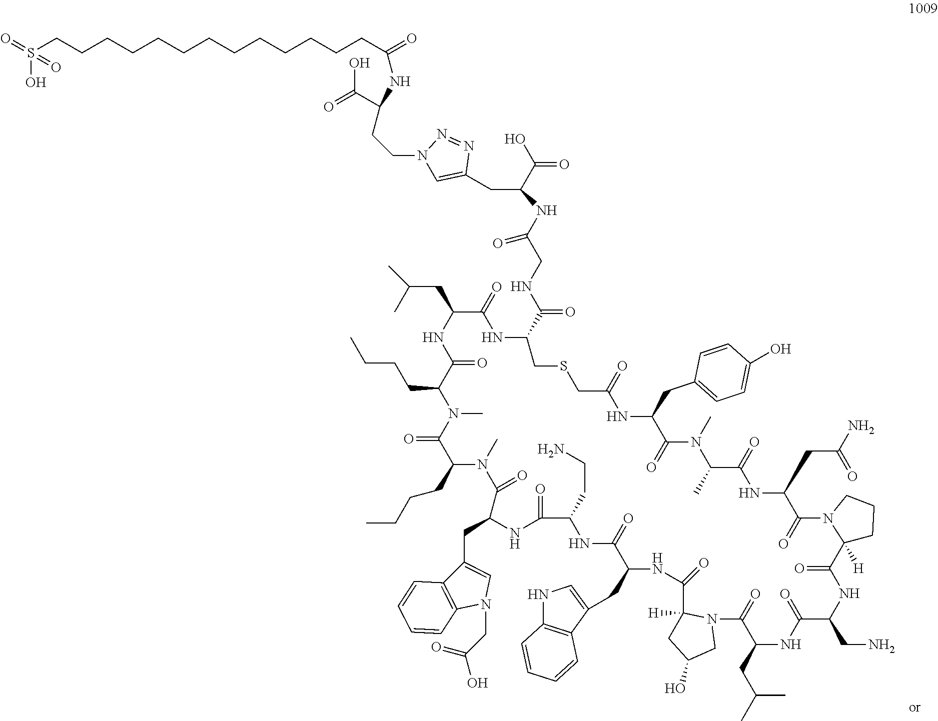 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CC[C@H](NC(=O)CCCCCCCCCCCCCS(=O)(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

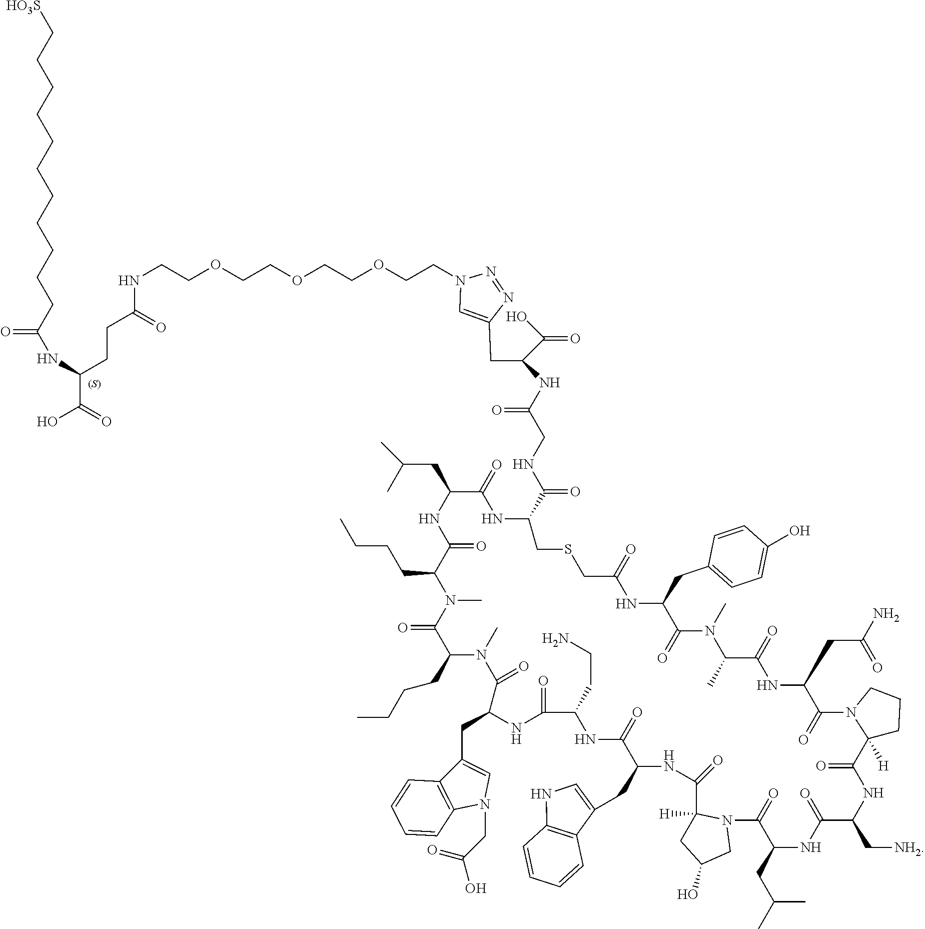

11. A pharmaceutical composition comprising a compound of claim 1, or a pharmaceutically acceptable salt thereof, and a pharmaceutically acceptable carrier.

12. A pharmaceutical composition comprising a compound of claim 10 or a pharmaceutically acceptable salt thereof, and a pharmaceutically acceptable carrier.

13. A method of enhancing, stimulating, and/or increasing an immune response in a subject in need thereof, said method comprising administering to the subject a therapeutically effective amount of a compound of claim 1, or a pharmaceutically acceptable salt thereof.

14. A method of inhibiting growth, proliferation, or metastasis of cancer cells in a subject in need thereof, said method comprising administering to the subject a therapeutically effective amount a compound of claim 1, or a pharmaceutically acceptable salt thereof.

15. The method of claim 14 wherein the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

16. A method of treating an infectious disease in a subject in need thereof, the method comprising administering to the subject a therapeutically effective amount of a compound of claim 1, or a pharmaceutically acceptable salt thereof.

17. The method of claim 16 wherein the infectious disease is caused by a virus.

18. A method of treating septic shock in a subject in need thereof, the method comprising administering to the subject a therapeutically effective amount of a compound of claim 1, or a pharmaceutically acceptable salt thereof.

19. A method of blocking the interaction of PD-L1 with PD-1 and/or CD80 in a subject, said method comprising administering to the subject a therapeutically effective amount of a compound of claim 1, or a pharmaceutically acceptable salt thereof.


SEQUENCE LISTING (2 sequences)

SEQ ID NO: 1 - protein, 384 aa, Artificial Sequence
  Recombinant Human PD-1-Ig -- hPD1(25-167)-3S-IG
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGSPGGGGGREPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 237 aa, Artificial Sequence
  Recombinant Human PD-L1-TVMV-His (PD-L1-His) -- hPDL1(19-239)-TVMV-His
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGSSETVRFQGHHHHHH